(19) United States
(12) Patent Application Publication
(10) Pub. No.: US 2026/0199465 A1
(43) Pub. Date: Jul. 16, 2026

(54) T CELL RECEPTORS BINDING HPV-16 EPITOPES

(71) Applicant: REPERTOIRE IMMUNE MEDICINES, INC., Cambridge, MA (US)

(72) Inventors: Christine MCINNIS, Waltham, MA (US); Daniel PREGIBON, Newton, MA (US)

(21) Appl. No.: 19/136,699

(22) PCT Filed: Dec. 15, 2023
(86) PCT No.: PCT/US2023/084382

§ 371 Date: Jun. 6, 2025

Related U.S. Application Data
(60) Provisional application No. 63/387,851, filed on Dec. 16, 2022.

(51) Int. Cl. A61K 40/11; A61K 40/32; A61K 40/46; C07K 14/705

(52) U.S. Cl. CPC A61K 40/11; A61K 40/32; A61K 40/46; C07K 14/7051; C07K 2317/32; C07K 2317/565

(57) ABSTRACT

Provided herein are T cell receptors (TCRs) that bind HPV-16 T cell epitopes presented by cognate major histocompatibility complexes (MHCs). Also provided are engineered TCRs and engineered immune cells expressing such TCRs, and related CN pharmaceutical compositions and therapeutic uses.

### CROSS-REFERENCE TO RELATED APPLICATIONS

This application claims the priority benefit of U.S. Provisional Application No. 63/387,851 filed Dec. 16, 2022, the entire contents of which is herein incorporated by reference.

### REFERENCE TO SEQUENCE LISTING SUBMITTED ELECTRONICALLY

The content of the electronically submitted sequence listing (Name: 4985_034PC01_SequenceListing_ST26, created on Dec. 14, 2023 and having a size of 222,352 bytes) is herein incorporated by reference in its entirety.

### FIELD OF THE INVENTION

The disclosure relates to T cell receptors (TCRs) that bind HPV-16 T cell epitopes presented by cognate major histocompatibility complexes (MHCs). The disclosure also relates to engineered TCRs and engineered immune cells expressing such TCRs, and related pharmaceutical compositions and therapeutic uses.

### BACKGROUND

Cancer immunotherapies are treatments that harness the body's own immune responses against cancer cells. Cancer immunotherapies include but are not limited to cancer-targeted antibodies, immune cell engagers, cancer vaccines, adoptive immune cell transfer therapies, tumor-infecting (oncolytic) viruses, immune checkpoint inhibitors, cytokines, and immunogenicity enhancing adjuvants. Certain immunotherapies have shown therapeutic benefits for treating cancers. For example, the U.S. Food and Drug Administration (FDA) has approved KIMMTRAK® (tebentafusp-tebn), a TCR-based T cell engager targeting gp100 for treatment of unresectable or metastatic uveal melanoma, and PROVENGE® (sipuleucel-T), a dendritic cell (DC)-based cancer vaccine that includes DCs activated with a tumor-derived antigen, for treatment of metastatic hormone-refractory prostate cancer.

Human papillomavirus (HPV) infection is associated with various cancers. The HPV genome is composed of early (E) and late genes involved in different aspects of the viral life cycle. Historically, studies have focused on HPV16 E proteins E6 and E7 as targets of immunotherapy due to allele prevalence and the established role of E6 and E7 in neoplastic transformation. Despite limited early clinical successes targeting these antigens, durable complete tumor regression has been an elusive goal. Other HPV genes, such as E1 and E2, have been relatively understudied. HPV infection has been identified as a major risk factor for head and neck cancers, particularly within the oropharynx. HPV-associated oropharyngeal squamous cell carcinoma (OPSCC) is characterized by distinct biology and a heterogenous mutational and immune landscape compared to HPV-negative tumors. For patients who experience locoregional recurrence or distant metastatic spread, subsequent therapy options are limited. Immune checkpoint inhibitors have been approved for recurrent/metastatic (R/M) OPSCC patients, but response rates are generally low and overall survival remains poor.

Accordingly, there remains a need for new treatments for HPV-associated cancers.

### SUMMARY OF THE INVENTION

The present disclosure is based, in part, upon the identification and engineering of T cell receptors (TCRs) that bind HPV-16 T cell epitopes presented by cognate major histocompatibility complexes (MHCs). Specifically, public CDR3 motifs have been identified from multiple TCRs that bind two HPV-16 E2 epitopes, namely, YSKNKVWEV (SEQ ID NO: 1) presented by HLA-B*08:01 or QVDYYGLYY (SEQ ID NO: 59) presented by HLA-A*01:01. T cells expressing these TCRs can also be used in an immune cell therapy for treating HPV-16 positive cancer.

Accordingly, in one aspect, the present disclosure provides a TCR comprising an alpha chain variable domain (Vα) and a beta chain variable domain (Vβ), wherein the Va comprises a CDR3 sequence of SEQ ID NO: 3, 5, 7, 9, 11, 13, 15, 17, 19, 21, 23, 25, 27, or 29, and the Vβ comprises a CDR3 sequence of SEQ ID NO: 2, wherein the TCR binds an epitope comprising the amino acid sequence of YSKNKVWEV (SEQ ID NO: 1) presented by HLA-B*08:01. The CDR3 sequence in the Vβ can comprise the amino acid sequence of SEQ ID NO: 4, 6, 8, 10, 12, 14, 16, 18, 20, 22, 24, 26, 28, or 30.

In certain embodiments, the Vβ further comprises the CDR1 and CDR2 amino acid sequences of TRBV7-6. In certain embodiments, the Vβ comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of TRBV7-6.

In certain embodiments, the Vβ further comprises the CDR1 and CDR2 amino acid sequences of TRBV7-7. In certain embodiments, the Vβ comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of TRBV7-7.

In certain embodiments, the Vβ further comprises the CDR1 and CDR2 amino acid sequences of TRBV7-8. In certain embodiments, the Vβ comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of TRBV7-8.

In certain embodiments, the pairing Va can comprise a CDR3 amino acid sequence and the CDR1 and CDR2 amino acid sequences of the corresponding TRAV set forth in Table 1. In certain embodiments, the Va comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of the corresponding TRAV set forth in Table 1.

In another aspect, the present disclosure provides a TCR comprising a Va and a Vβ, wherein the Va comprises a CDR3 sequence of SEQ ID NO: 60, and the Vβ comprises a CDR3 sequence of SEQ ID NO: 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, or 134, wherein the TCR binds an epitope comprising the amino acid sequence of QVDYYGLYY (SEQ ID NO: 59) presented by HLA-A*01:01. The CDR3 sequence in the Va can comprise the amino acid sequence of SEQ ID NO: 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133.

In certain embodiments, the Va further comprises the CDR1 and CDR2 amino acid sequences of TRAV21. In certain embodiments, the Va comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of TRAV21.

In certain embodiments, the pairing Vβ comprises a CDR3 amino acid sequence and the CDR1 and CDR2 amino acid sequences of the corresponding TRBV set forth in Table 2. In certain embodiments, the Vβ comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of the corresponding TRBV set forth in Table 2.

Any of the TCRs disclosed herein can be isolated, non-naturally occurring, and/or engineered. For example, the TCR can be a soluble TCR. The soluble TCR can further comprise an alpha chain constant domain (Ca) and a beta chain constant domain (Cβ). In certain embodiments, the soluble TCR comprises one or more mutations that stabilize the interaction between the Cα and the Cβ. In certain embodiments, the Cα comprises a cysteine residue at position 48 corresponding to the TRAC amino acid sequence of SEQ ID NO: 209, and the Cβ comprises a cysteine residue at position 57 corresponding to the TRBC1 or TRBC2 amino acid sequence of SEQ ID NO: 210 or 211, respectively. The cysteine residue at position 48 in the Cα and the cysteine residue at position 57 in the Cβ can form a disulfide bond. In certain embodiments, in the soluble TCR, (a) the Cα comprises a phenylalanine residue at position 21, an isoleucine residue at position 32, and/or a threonine residue at position 72, corresponding to the TRAC amino acid sequence of SEQ ID NO: 209; and/or (b) the Cβ comprises a lysine residue at position 18, an arginine residue at position 23, a proline residue at position 39, and/or an aspartic acid or glutamic acid at position 54, corresponding to the TRBC1 or TRBC2 amino acid sequence of SEQ ID NO: 210 or 211, respectively.

The present disclosure also provides a TCR fusion protein comprising a soluble TCR disclosed herein and a binding domain that binds a receptor on an outer surface of an immune cell, a cytotoxic agent, a detectable label, or a combination thereof. For example, the binding domain can bind a receptor on an outer surface of a T cell (e.g., CD3, CD2, CD28, or CD8). In certain embodiments, the binding domain comprises an antibody, or an antigen-binding fragment thereof, that binds the receptor. In certain embodiment, the binding domain comprises a heavy chain variable domain (VH) and a light chain variable domain (VL) of an anti-CD3 antibody.

The TCR fusion protein disclosed herein can further comprise a half-life extending domain, for example, an antibody Fc region. In certain embodiments, the antibody Fc region comprises a human IgG1 Fc region comprising one or more effector function silencing mutations, optionally at one or more of positions selected from 233, 234, 235, 236, 297, 327, 330, and 331, according to EU numbering. In certain embodiments, the antibody Fc region comprises a human IgG4 or IgG2 Fc region.

The present disclosure also provides a pharmaceutical composition comprising a TCR fusion protein disclosed herein and a pharmaceutically acceptable carrier or excipient.

The present disclosure also provides a method of treating cancer, the method comprising administering to a subject in need thereof an effective amount of a TCR fusion protein or pharmaceutical composition disclosed herein.

The present disclosure also provides one or more nucleic acids encoding a TCR, soluble TCR, or TCR fusion protein disclosed herein. In addition, the present disclosure provides a vector comprising the one or more nucleic acids. Further, the present disclosure provides a cell comprising the one or more nucleic acids or the vector. Further, the present disclosure provides a method of producing a TCR or TCR fusion protein, the method comprising incubating the cell under conditions to express the TCR or the TCR fusion protein.

In another aspect, the present disclosure provides an engineered immune cell comprising one or more exogenous nucleic acids that encode a TCR disclosed herein. The immune cell can be a T cell, for example, a CD8+ T cell. The present disclosure also provides a pharmaceutical composition comprising the engineered immune cell and a pharmaceutically acceptable carrier or excipient.

In addition, the present disclosure provides a method of producing an engineered immune cell, the method comprising contacting an immune cell with one or more nucleic acids that encode the TCR.

Further, the present disclosure provides a method of treating cancer, the method comprising administering to a subject in need thereof an effective amount of the engineered immune cell of or the pharmaceutical composition. The immune cell can be autologous or obtained from a healthy donor.

With respect to all the methods of treating cancer disclosed herein, it is contemplated that the cancer can be HPV-16 positive, for example, HPV-16 E2 positive.

### BRIEF DESCRIPTION OF THE DRAWINGS

FIG. 1 shows an illustration of the DECODE workflow.

FIGS. 2A-F show the characterization of immune and non-immune cell infiltrates in HPV+ OPSCC tumors. FIG. 2A shows a uMAP clustering analysis showing different cell types in HPV+ dissociated tumor samples. FIG. 2B shows a distribution of immune and non-immune cell types in the dissociated tumors from recurrent vs non-recurrent patients. FIGS. 2C-F show uMAP representations of B cell (FIG. 2C) and T cell re-clustering (FIG. 2E) along with quantitative analysis of B cell (FIG. 2D) T cell subsets (FIG. 2F) in HPV+ patients (n=14). FIG. 2C shows a differential distribution of B cells in multiple patient tumors (n=13) screened by 10× single cell RNA sequencing.

FIGS. 3A-B show graphs for the correlation of HPV gene expression of antigens from HPV 16 and HPV33 with clinical variables and T cell subsets.

FIG. 4A-4D show the relationship between HPV gene expression and tumor stage, recurrence status, or smoking status. The correlations between HPV16 and HPV33 gene expression and various T cell subsets are shown in FIG. 3B. FIGS. 4A-D shows an exemplary analysis of HPV16-specific T-cell responses in treatment-naïve head and neck cancer patients. FIG. 4A shows a heatmap of T-cells from selected patients reacting to HPV16 E1, E2, E4, E5, E6, and E7. FIG. 4B shows the frequency of CD8 and CD4 T cell response against different HPV16 antigens by HLA allele. FIG. 4C-D shows a uMAPs illustration of HPV antigens reactive CD8 (FIG. 4C) and CD4 T cells (FIG. 4D) and the expression of genes associated with tumor reactivity, cytotoxicity and exhaustion.

FIGS. 5A-F show HPV strain-specific differences in proportion of different immune cells. FIG. 5A shows a graph of the percentage of different immune cells in HPV16 (blue) and HPV33 positive tumors (purple). FIG. 5B shows a graph of the percentage of different T cell subsets in HPV16 (blue) and HPV33 positive tumors (purple). FIG. 5C shows gene expression differences in B-cells between HPV16 and HPV33+ patients. FIG. 5D shows a graph of the A*02:01 frequency present in HPV33+ patients compared to the US Caucasian population (Source: www.allelefrequencies.net). FIG. 5E shows a graph of a comparative analysis E7:E6 gene expression in A*02:01 HPV33+ versus HPV16+ or HPV33+A*02:01—patient tumors. FIG. 5F shows a graph of the predicted binding affinities of predicted epitopes across HPV16 E7 and HPV33 E7. Data in FIGS. 5A, 5B, and 5C are shown as mean±SEM. Statistical significance was analyzed by performing Student's t-test. p<0.05 is considered significant.

FIGS. 6A-6B show exemplary public TCR clonotypes responding to presentation of HPV16 E2 QVDYYGLYY and HPV16 E2 YSKNKVWEV peptides. FIG. 6A shows a consensus sequence homology motif for the CDR3 of the alpha and the beta chains for T cells analyzed of HPV16 E2 QVDYYGLYY-A*01:01—specific T-cells and their clonotypes across patients, a graph of an analysis of T-cells from several different patients appearing in the cluster in FIG. 6A, with their contributions from DFCI1, DFCI11, DFCI20 and DFCI18, and validation of these clonotypes in an orthogonal assay. The graph of an analysis of T cell activation for T cells with TCRs with beta chains homologous to QVDYYGLYY-reactive T-cells in FIG. 6A (beta homology) and T cells with TCRs with similar alpha chains are shown in (alpha homology) as measured by % CD69 induction by a cognate peptide presented by an APC compared to DMSO control. Data represents mean±SD. (FIG. 6B) shows a consensus sequence homology motif for the CDR3 of the alpha and the beta chains for T cells analyzed of HPV16 E2 YSKNKVWEV-B*08:01—specific T-cells and their clonotypes across patients, a graph of an analysis of T-cells from several different patients appearing in the cluster in FIG. 6B, with their contributions from DFCI1, DFCI11, DFCI20 and DFCI18, and validation of these clonotypes in an orthogonal assay. The graph of an analysis of T cell activation for T cells with TCRs with beta chains homologous to YSKNKVWEV -reactive T-cells in FIG. 6B (beta homology) as measured by % CD69 induction by a cognate peptide presented by an APC compared to DMSO control. Data represents mean±SD.

FIG. 7 shows a bar graph of T cell activation as measured by % CD69 induction by a cognate peptide presented by an APC compared to DMSO control. Data represents mean±SD.

### DETAILED DESCRIPTION

The present disclosure is based, in part, upon the identification and engineering of T cell receptors (TCRs) that bind HPV-16 T cell epitopes presented by cognate major histocompatibility complexes (MHCs). Specifically, public CDR3 motifs have been identified from multiple TCRs that bind two HPV-16 E2 epitopes, namely, YSKNKVWEV (SEQ ID NO: 1) presented by HLA-B*08:01 or QVDYYGLYY (SEQ ID NO: 59) presented by HLA-A*01:01. T cells expressing these TCRs can also be used in an immune cell therapy for treating HPV-16 positive cancer.

### I. Definitions

To facilitate an understanding of the present invention, a number of terms and phrases are defined below.

As used herein, the terms “a” and “an” mean “one or more” and include the plural unless the context is inappropriate.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

As used herein, the expression “and/or” in connection with two or more recited objects includes individually each of the recited objects and the various combinations of two or more of the recited objects, unless otherwise understood from the context and use.

As used herein, the terms “antigen-presenting cell” or “APC” refer to a cell or particle that elicits a cellular immune response by displaying a T cell epitope presented by a major histocompatibility complex (MHC) on an outer surface of the cell or particle, for recognition by an immune cell such as a T cell. APCs include, e.g., professional APCs, such as dendritic cells, macrophages, Langerhans cells, and B cells, that express both class I and class II MHCs, and non-professional APCs (e.g., nucleated cells) that generally express only class I MHCs. APCs also include artificial APCs (aAPCs), e.g., cells (e.g., drosophila cells) engineered to express an MHC that presents a T cell epitope. In addition, artificial APCs include particles (e.g., beads, nanoparticles, or liposomes) that present a peptide-MHC on the surface, configured such that the aAPC can prime or stimulate T cells in vitro, ex vivo, or in vivo. Exemplary aAPCs are known in the art and are described in Section V herein.

As used herein, the term “effective amount” refers to the amount of a compound (e.g., a compound of the present disclosure) sufficient to effect beneficial or desired results. An effective amount can be administered in one or more administrations, applications or dosages and is not intended to be limited to a particular formulation or administration route.

As used herein, percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Similarly, percent “identity” between a nucleic acid sequence and a reference sequence is defined as the percentage of nucleotides in the nucleic acid sequence that are identical to the nucleotides in the reference sequence, after aligning the sequences and introducing gaps, if necessary, to achieve the maximum percent sequence identity. Alignment for purposes of determining percent sequence identity (e.g., amino acid sequence identity or nucleic acid sequence identity) can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

As used herein, the term “isolated” when used in reference to a biological substance means that the substance is not in its native state or is removed from at least a portion of other molecules associated or occurring with the substance in its native environment (e.g., within a cell or tissue) or in another environment (e.g., in a cell extract, extraction buffer, etc.). An isolated biological substance, e.g., a protein, peptide or cell, can be essentially free of other biological molecules. For example, in certain embodiments, an isolated protein, peptide or cell can be at least 40%, 50%, 60%, 70%, 80%, 90%, 95%, 96%, 97%, 98%, 99% or 100% free of other molecules present in a natural or non-natural environment. For example, in certain embodiments, an isolated TCR is not present in a native composition of the TCR, e.g., on the surface of a T cell.

The term “major histocompatibility complex” or “MHC” refers to a genomic locus containing a group of genes that encode the polymorphic cell-membrane-bound glycoproteins known as MHC class I and class II molecules, which regulate the immune response by presenting peptides of fragmented proteins to CD8⁺ (e.g., cytotoxic) and CD4⁺ (e.g., helper) T lymphocytes, respectively. In humans, this group of genes is also called “human leukocyte antigen” or “HLA.” Human MHC class I genes encode, for example, HLA-A, HLA-B, and HLA-C proteins. Class I HLA protein is a heterodimer composed of a heavy α-chain and a smaller β-chain. The α-chain is encoded by a variant HLA gene, and the β chain is an invariant β2 microglobulin (β2m) polypeptide encoded by a separate region of the human genome. Human MHC class II genes encode, for example, HLA-DM, HLA-DO, HLA-DP, HLA-DQ, and HLA-DR proteins. Class II HLA protein is a heterodimer composed of a and β chains both encoded by variant HLA genes. A HLA can include multiple serotypes (e.g., HLA-A*02, HLA-DPA1*02) which each may include multiple alleles (e.g., HLA-A*02:01, HLA-DPA1*02:02). Nucleotide sequences and a gene map of human MHC are publicly available (e.g., The MHC sequencing consortium, Nature 401:921-923, 1999). The terms “major histocompatibility complex” and “MHC” also refer to the polymorphic glycoproteins encoded by the MHC class I or class II genes, where appropriate in the context, and proteins comprising variants thereof that bind T cell epitopes (e.g., class I or class II epitopes). Generally, a class I MHC binds a T cell epitope in a groove formed by its al domain and α2 domain and a class II MHC binds a T cell epitope in a groove formed by its al domain and β1 domain. The term “soluble MHC” refers to an extracellular fragment of a MHC comprising corresponding α1 and α2 domains that bind a class I T cell epitope or corresponding α1 and β1 domains that bind a class II T cell epitope, where the α1 and α2 domains or the al and β1 domains are derived from a naturally occurring MHC or a variant thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Adejare, Remington, The Science and Practice of Pharmacy (23rd Ed. 2020).

As used herein, the terms “prime” and “priming,” in the context of antigen presenting cells and T cells, refers to naïve T cell clonal expansion, activation, differentiation, and/or generation of memory T cells as a result of TCR engagement by a T cell epitope presented by a MHC on an outer surface of an antigen presenting cell.

As used herein, the terms “subject” and “patient” are used interchangeably and refer to an organism to be treated by the methods and compositions described herein. Such organisms preferably include, but are not limited to, mammals (e.g., murines, simians, equines, bovines, porcines, canines, felines, and the like), and more preferably include humans.

As used herein, a cell composition is “substantially free” of a specified type of cells if this type of cells does not exceed 5%, 2%, 1%, 0.1%, 0.01%, or 0.001% of the total amount of cells in the composition. The percentage of each cell type in a cell composition can be determined using a method known in the art. For example, immune cells are often characterized by cell type-specific markers (e.g., cell surface markers) and the amount of each cell type can be determined by flow cytometry.

As used herein, the term “T cell epitope” refers to a peptide of an antigen protein presentable by a class I or class II MHC. T cell epitopes presentable by class I MHC proteins (e.g., HLA-A, HLA-B) are typically 8-11 amino acids in length. The peptide is located in the peptide-binding groove of the central region of the α1/α2 domains of the class I MHC. T cell epitopes presentable by class II MHC proteins are typically 13-25 amino acids (e.g., 15-24 amino acids) in length. The peptide is located in the peptide-binding groove formed by the α1/β1 domains of the class II MHC. A T cell epitope is “immunogenic” if it stimulates a T cell response, for example, a stronger and/or longer-lasting T cell response than a reference T cell epitope in a subject. Immunogenicity can be measured by proliferation, activation, differentiation, and/or memory formation of T cells when contacted with the T cell epitope presented by a cognate MHC. Immunogenicity can also be assessed based on the amount of total T cells, effector T cells (e.g., cytotoxic T cells or helper T cells), and/or memory T cells reactive to the T cell epitope in a subject. The strength and duration of the T cell response (e.g., proliferation or activation) can be specific to the subject's MHC serotypes and TCR repertoire. Immunogenic T cell epitopes can also be identified in a population of organisms (e.g., humans) with respect to its average strength and duration of T cell response to the T cell epitopes.

As used herein, the terms “T cell receptor” and “TCR” refer to a surface protein (e.g., a heterodimeric protein) of a T cell that allows the T cell to recognize an antigen and/or an epitope thereof, typically presented by a major histocompatibility complex (MHC), or a fragment of such surface protein comprising at least its variable domains. Typically, TCRs are heterodimers comprising two different protein chains. In many T cells, the TCR comprises an alpha (α) chain and a beta (β) chain. Each chain, in its native form, typically comprises two extracellular domains, a variable (V) domain and a constant (C) domain, the latter of which is membrane-proximal. The variable domain of α-chain (Vα) and the variable domain of β-chain (Vβ) each comprise three hypervariable regions that are also referred to as the complementarity determining regions (CDRs) such as CDR1, CDR2, and CDR3. The CDRs, in particular CDR3, are primarily responsible for contacting epitopes and thus define the specificity of the TCR, although, under certain circumstances, the CDR1 of the α-chain can interact with the N-terminal part of the antigen, and CDR1 of the 3-chain interacts with the C-terminal part of the antigen. All numbering of the amino acid sequences and designation of protein loops and sheets of TCRs is according to the IMGT numbering scheme (the international ImMunoGeneTics information system; Lefranc et al., (2003) Dev Comp Immunol 27:55 77; Lefranc et al. (2005) Dev Comp Immunol 29:185-203). The terms “T cell receptor” and “TCR” also include an “engineered T cell receptor” or “engineered TCR,” such as a recombinantly modified protein comprising a fragment of a naturally occurring TCR that bind a T cell epitope-MHC complex, or a variant of such fragment. For example, an engineered TCR may contain a modified binding cassette (e.g., where one or more CDR sequences or other elements is modified, for example, by introducing corresponding sequences from a different TCR). For example, the α and/or β chain CDR3 sequences of a first TCR identified herein may be introduced into a second, different TCR present in or derived from a given T cell. The TCR may also contain modification, truncation, or deletion of its constant region, hinge region, transmembrane region, and/or intracellular region. For example, at least the transmembrane region and the intracellular region can be deleted to generate a soluble TCR. According, in certain embodiments, a TCR (e.g., engineered TCR) comprises corresponding Vα and Vβ domains that bind a T cell epitope-MHC complex, where the Vα and Vβ domains are derived from a naturally occurring TCR or a variant thereof.

As used herein, the term “treating” includes any effect, e.g., lessening, reducing, modulating, ameliorating or eliminating, that results in the improvement of the condition, disease, disorder, and the like, or ameliorating a symptom thereof.

As a general matter, compositions specifying a percentage are by weight unless otherwise specified. Further, if a variable is not accompanied by a definition, then the previous definition of the variable controls.

### II. Tcr Constructs

The present disclosure provides a TCR (e.g., isolated TCR) that binds a T cell epitope. An αβ TCR generally includes an α-chain variable domain (Vα) comprising CDR1α, CDR2α, and CDR3α, and a β-chain variable domain (Vβ) comprising CDR1β, CDR3β, and CDR3β. The CDR1 and CDR2 sequences are encoded by the germline V genes, whereas the hypervariable CDR3 sequences are encoded by the V-J (α-chain) or V-D-J (β-chain) regions that undergo somatic rearrangement. Without wishing to be bound by theory, it is understood that the CDR3 regions generally contact the peptide, and the CDR1 and CDR2 regions generally contact the MHC (see, e.g., Garcia and Adams, Cell (2005) 122(3):333-36). Accordingly, it is contemplated that the antigen specificity of a TCR largely depends upon the CDR3 sequences of the TCR. In certain embodiments, the TCR of the present disclosure binds to an epitope comprising the amino acid sequence of YSKNKVWEV (SEQ ID NO: 1) presented by HLA-B*08:01 and comprises corresponding CDR3α and CDR3β amino acid sequences set forth in Table 1. In certain embodiments, the TCR of the present disclosure binds to an epitope comprising the amino acid sequence of QVDYYGLYY (SEQ ID NO: 59) presented by HLA-A*01:01 and comprises corresponding CDR3α and CDR3β amino acid sequences set forth in Table 2.

Public motifs of CDR3 sequences are disclosed herein. A public motif is a TCR motif (with identical or homologous sequences) shared by TCR sequences from multiple individuals, when mounting a T cell response to the same antigenic epitope. TCRs shared across multiple individuals are biased for usage of particular V-genes and conserved CDR3α or CDR3β motifs. In certain embodiments, the CDR3α or CDR3β amino acid sequence of a TCR disclosed herein fall within a CDR3α or CDR3β motif consensus sequence.

In certain embodiments, the TCR of the present disclosure binds to an epitope comprising the amino acid sequence of YSKNKVWEV (SEQ ID NO: 1) presented by HLA-B*08:01, and the Vβ comprises a CDR3 sequence of CASSX1X2X3RX4X5X6QX7F, wherein X1 is L, V, I, S, or a peptide bond (no amino acid residue); X2 is R or F; X3 is A, K, R, or a peptide bond (no amino acid residue); X4 is D or N; X5 is Q or W; X6 is P or T; and X7 is H or Y (SEQ ID NO: 2). In certain embodiments, X1 is L, V, I, or S. In certain embodiments, X3 is an amino acid residue. The pairing Vα can comprises a CDR3 sequence of SEQ ID NO: 3, 5, 7, 9, 11, 13, 15, 17, 19, 21, 23, 25, 27, or 29.

In certain embodiments, the Vβ comprises a CDR3 sequence of SEQ ID NO: 4, 6, 8, 10, 12, 14, 16, 18, 20, 22, 24, 26, 28, or 30. In certain embodiments, the Vα and Vβ of a TCR disclosed herein comprise CDR3 sequences of 3 and 4, 5 and 6, 7 and 8, 9 and 10, 11 and 12, 13 and 14, 15 and 16, 17 and 18, 19 and 20, 21 and 22, 23 and 24, 25 and 26, 27 and 28, or 29 and 30, respectively.

In certain embodiments, the Vβ further comprises the CDR1 and CDR2 amino acid sequences of TRBV7-6. In certain embodiments, the Vβ comprises an amino acid sequence at least 90% identical to the amino acid sequence of TRBV7-6. In certain embodiments, the Vβ further comprises the CDR1 and CDR2 amino acid sequences of TRBV7-7. In certain embodiments, the Vβ comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of TRBV7-7. In certain embodiments, the Vβ further comprises the CDR1 and CDR2 amino acid sequences of TRBV7-8. In certain embodiments, the Vβ comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of TRBV7-8. In certain embodiments, the Vα comprises a CDR3 amino acid sequence and the CDR1 and CDR2 amino acid sequences of the corresponding TRAV set forth in Table 1. In certain embodiments, the Vα comprises an amino acid sequence at least 90% identical to the amino acid sequence of the corresponding TRAV set forth in Table 1.

TABLE 1
Exemplary TCRs that bind YSKNKVWEV (SEQ ID NO: 1)
presented by HLA-B*08:01

| | | | Junction | | | CDR3 | |
|---|---|---|---|---|---|---|---|
| | | | | SEQ | | | SEQ |
| TCR | TCR | | Nucleotide | ID | J | Amino Acid | ID |
| ID | Chain | V gene | Sequence | NO | gene | Sequence | NO |
| TCR_ | Alpha | TRAV14/ | TGTGCAATGAGAGAGG | 31 | TRAJ40 | CAMREGGTSGTY | 3 |
| YSK_ | | DV4 | GCGGTACCTCAGGAAC | | | KYIF | |
| 1 | | | CTACAAATACATCTTT | | | | |
| | Beta | TRBV7-6 | TGTGCCAGCAGCTTAC | 32 | TRBJ1- | CASSLRRDQPQH | 4 |
| | | | GACGCGATCAGCCCCA | | 5 | F | |
| | | | GCATTTT | | | | |
| TCR_ | Alpha | TRAV14/ | TGTGCAATGAGAGAGG | 33 | TRAJ12 | CAMREDAMDSSY | 5 |
| YSK_ | | DV4 | ATGCGATGGATAGCAG | | | KLIF | |
| 2 | | | CTATAAATTGATCTTC | | | | |
| | Beta | TRBV7-6 | TGTGCCAGCAGCTTGA | 34 | TRBJ1- | CASSLRRDQPQH | 6 |
| | | | GGAGAGATCAGCCCCA | | 5 | F | |
| | | | GCATTTT | | | | |
| TCR_ | Alpha | TRAV14/ | TGTGCAATGAGAGAGG | 35 | TRAJ40 | CAMREASLSGTY | 7 |
| YSK_ | | DV4 | CTTCCCTCTCAGGAAC | | | KYIF | |
| 3 | | | CTACAAATACATCTTT | | | | |
| | Beta | TRBV7-6 | TGTGCCAGCAGCTTCA | 36 | TRBJ1- | CASSFKRDQPQH | 8 |
| | | | AGAGGGATCAGCCCCA | | 5 | F | |
| | | | GCATTTT | | | | |
| TCR_ | Alpha | TRAV14/ | TGTGCAATGAGAGAGG | 37 | TRAJ40 | CAMREGSLQGTY | 9 |
| YSK_ | | DV4 | GCAGTCTACAGGGAAC | | | KYIF | |
| 4 | | | CTACAAATACATCTTT | | | | |
| | Beta | TRBV7-7 | TGTGCCAGCAGCATAC | 38 | TRBJ1- | CASSIRRDQPQH | 10 |
| | | | GCCGGGATCAGCCCCA | | 5 | F | |
| | | | GCATTTT | | | | |
| TCR_ | Alpha | TRAV14/ | TGTGCAATGAGAGGGG | 39 | TRAJ12 | CAMRGVMDSSYK | 11 |
| YSK_ | | DV4 | TGATGGATAGCAGCTA | | | LIF | |
| 5 | | | TAAATTGATCTTC | | | | |
| | Beta | TRBV7-6 | TGTGCCAGCAGCTTAC | 40 | TRBJ1- | CASSLRRDQPQH | 12 |
| | | | GGAGGGATCAGCCCCA | | 5 | F | |
| | | | GCATTTT | | | | |
| TCR_ | Alpha | TRAV13-2 | TGTGCAGAGAATATGG | 41 | TRAJ29 | CAENMDGNTPLV | 13 |
| YSK_ | | | ACGGAAACACACCTCT | | | F | |
| 6 | | | TGTCTTT | | | | |
| | Beta | TRBV7-6 | TGTGCCAGCAGCTTAA | 42 | TRBJ1- | CASSLRRDQPQH | 14 |
| | | | GGAGAGATCAGCCCCA | | 5 | F | |
| | | | GCATTTT | | | | |
| TCR_ | Alpha | TRAV14/ | TGTGCAATGAGAGAGG | 43 | TRAJ40 | CAMREGGTSGTY | 15 |
| YSK_ | | DV4 | GCGGTACCTCAGGAAC | | | KYIF | |
| 7 | | | CTACAAATACATCTTT | | | | |
| | Beta | TRBV7-6 | TGTGCCAGCAGCTTAC | 44 | TRBJ1- | CASSLRRDQPQH | 16 |
| | | | GTCGGGATCAGCCCCA | | 5 | F | |
| | | | GCATTTT | | | | |
| TCR_ | Alpha | TRAV14/ | TGTGCAATGAGAGAGG | 45 | TRAJ40 | CAMREGSTSGTY | 17 |
| YSK_ | | DV4 | GTAGTACCTCAGGAAC | | | KYIF | |
| 8 | | | CTACAAATACATCTTT | | | | |
| | Beta | TRBV7-6 | TGTGCCAGCAGCTCAC | 46 | TRBJ1- | CASSSRRDQPQH | 18 |
| | | | GCAGAGATCAGCCCCA | | 5 | F | |
| | | | GCATTTT | | | | |
| TCR_ | Alpha | TRAV19 | TGTGCTCTGAGCGGGC | 47 | TRAJ12 | CALSGQMDSSYK | 19 |
| YSK_ | | | AGATGGATAGCAGCTA | | | LIF | |
| 9 | | | TAAATTGATCTTC | | | | |
| | Beta | TRBV7-6 | TGTGCCAGCAGCGTAC | 48 | TRBJ1- | CASSVRRDQPQH | 20 |
| | | | GCAGGGATCAGCCCCA | | 5 | F | |
| | | | GCATTTT | | | | |
| TCR_ | Alpha | TRAV27 | TGTGCAGGAGCTAGCG | 49 | TRAJ20 | CAGASDYKLSF | 21 |
| YSK_ | | | ACTACAAGCTCAGCTT | | | | |
| 10 | | | T | | | | |
| | Beta | TRBV7-6 | TGTGCCAGCAGCCGGC | 50 | TRBJ1- | CASSRRRNQPQH | 22 |
| | | | GCCGAAATCAGCCCCA | | 5 | F | |
| | | | GCATTTT | | | | |
| TCR_ | Alpha | TRAV19 | TGTGCTCTGAGTGGGA | 51 | TRAJ12 | CALSGTMDSSYK | 23 |
| YSK_ | | | CTATGGATAGCAGCTA | | | LIF | |
| 12 | | | TAAATTGATCTTC | | | | |
| | Beta | TRBV7-6 | TGTGCCAGCAGCTTAC | 52 | TRBJ1- | CASSLRRDWPQH | 24 |
| | | | GGAGGGACTGGCCCCA | | 5 | F | |
| | | | GCATTTT | | | | |
| TCR_ | Alpha | TRAV9-2 | TGTGCTCTGACTTTTA | 53 | TRAJ23 | CALTFNQGGKLI | 25 |
| YSK_ | | | ACCAGGGAGGAAAGCT | | | F | |
| 12 | | | TATCTTC | | | | |
| | Beta | TRBV7-8 | TGTGCCAGCAGCTTAC | 54 | TRBJ1- | CASSLRARDQPQ | 26 |
| | | | GGGCAAGGGATCAGCC | | 5 | HF | |
| | | | CCAGCATTTT | | | | |
| TCR_ | Alpha | TRAV14/ | TGTGCAATGAGAGAGG | 55 | TRAJ12 | CAMREGGSDSSY | 27 |
| YSK_ | | DV4 | GCGGGTCGGATAGCAG | | | KLIF | |
| 13 | | | CTATAAATTGATCTTC | | | | |
| | Beta | TRBV7-6 | TGTGCCAGCAGCTTAA | 56 | TRBJ2- | CASSLRRDQTQY | 28 |
| | | | GGCGGGACCAGACCCA | | 5 | F | |
| | | | GTACTTC | | | | |
| TCR_ | Alpha | TRAV14/ | TGTGCAATGAGAGAGG | 57 | TRAJ40 | CAMREGGTSGTY | 29 |
| YSK_ | | DV4 | GCGGGACCTCAGGAAC | | | KYIF | |
| 14 | | | CTACAAATACATCTTT | | | | |
| | Beta | TRBV7-6 | TGTGCCAGCAGCTTAA | 58 | TRBJ1- | CASSLRRDQPQH | 30 |
| | | | GAAGGGACCAGCCCCA | | 5 | F | |
| | | | GCATTTT | | | | |

In certain embodiments, the TCR of the present disclosure binds to an epitope comprising the amino acid sequence of QVDYYGLYY (SEQ ID NO: 59) presented by HLA-A*01:01, and the Vβ comprises a CDR3 sequence of CAXIX2TGGFKTIF, wherein X1 is V or P; X2 is D or N (SEQ ID NO: 60). The pairing Vβ can comprises a CDR3 sequence of SEQ ID NO: 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, or 134.

In certain embodiments, the Vα comprises a CDR3 sequence of SEQ ID NO: 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133. In certain embodiments, the Vα and Vβ of a TCR disclosed herein comprise CDR3 sequences of 61 and 62, 63 and 64, 65 and 66, 67 and 68, 69 and 70, 71 and 72, 73 and 74, 75 and 76, 77 and 78, 79 and 80, 81 and 82, 83 and 84, 85 and 86, 87 and 88, 89 and 90, 91 and 92, 93 and 94, 95 and 96, 97 and 98, 99 and 100, 101 and 102, 103 and 104, 105 and 106, 107 and 108, 109 and 110, 111 and 112, 113 and 114, 115 and 116, 117 and 118, 119 and 120, 121 and 122, 123 and 124, 125 and 126, 127 and 128, 129 and 130, 131 and 132, or 133 and 134, respectively.

In certain embodiments, the Vα further comprises the CDR1 and CDR2 amino acid sequences of TRAV21. In certain embodiments, the Vα comprises an amino acid sequence at least 90% (e.g., at least 91%, at least 92%, at least 93%, at least 94%, at least 95%, at least 96%, at least 97% at least 98, at least 99% or 100%) identical to the amino acid sequence of TRAV21. In certain embodiments, the Vβ comprises a CDR3 amino acid sequence and the CDR1 and CDR2 amino acid sequences of the corresponding TRBV set forth in Table 2. In certain embodiments, the Vβ comprises an amino acid sequence at least 90% (e.g., at least 91%, at least 920%, at least 93% at least 94% at least 95% at least 960%, at least 97% at least 980%, at least 99%, or 100%) identical to the amino acid sequence of the corresponding TRBV set forth in Table 2.

TABLE 2
Exemplary TCRs that bind QVDYYGLYY (SEQ ID NO: 59)
presented by HLA-A*01:01

| | | | Junction | | | CDR3 | |
|---|---|---|---|---|---|---|---|
| | | | | SEQ | | | SEQ |
| TCR | TCR | | Nucleotide | ID | | Amino Acid | ID |
| ID | Chain | V gene | Sequence | NO | J gene | Sequence | NO |
| TCR_ | Alpha | TRAV21 | TGTGCTGTCGATACTGGAGG | 135 | TRAJ9 | CAVDTGGFKTIF | 61 |
| QVD_ | | | CTTCAAAACTATCTTT | | | | |
| 1 | Beta | TRBV27 | TGTGCCAGCAGTCCCGCGGA | 136 | TRBJ1- | CASSPADRGDTI | 62 |
| | | | CAGGGGTGACACCATATATT | | 3 | YF | |
| | | | TT | | | | |
| TCR_ | Alpha | TRAV21 | TGTGCTGTCAATACTGGAGG | 137 | TRAJ9 | CAVNTGGFKTIF | 63 |
| QVD_ | | | CTTCAAAACTATCTTT | | | | |
| 2 | Beta | TRBV27 | TGTGCCAGCAGTGGAACAGG | 138 | TRBJ1- | CASSGTGMANEK | 64 |
| | | | GATGGCTAATGAAAAACTGT | | 4 | LFF | |
| | | | TTTTT | | | | |
| TCR_ | Alpha | TRAV21 | TGTGCTGTGGATACTGGAGG | 139 | TRAJ9 | CAVDTGGFKTIF | 65 |
| QVD_ | | | CTTCAAAACTATCTTT | | | | |
| 3 | Beta | TRBV27 | TGTGCCAGCAGTTTGGCCTC | 140 | TRBJ1- | CASSLASGDYGY | 66 |
| | | | TGGGGATTATGGCTACACCT | | 2 | TF | |
| | | | TC | | | | |
| TCR_ | Alpha | TRAV21 | TGTGCTGTTGATACTGGAGG | 141 | TRAJ9 | CAVDTGGFKTIF | 67 |
| QVD_ | | | CTTCAAAACTATCTTT | | | | |
| 4 | Beta | TRBV27 | TGTGCCAGCAGTTCGGCGGA | 142 | TRBJ1- | CASSSADGAGYT | 68 |
| | | | CGGCGCGGGCTACACCTTC | | 2 | F | |
| TCR_ | Alpha | TRAV21 | TGTGCTGTGGATACTGGAGG | 143 | TRAJ9 | CAVDTGGFKTIF | 69 |
| QVD_ | | | CTTCAAAACTATCTTT | | | | |
| 5 | Beta | TRBV27 | TGTGCCAGCAGTTTGGCAGA | 144 | TRBJ1- | CASSLADGNEKL | 70 |
| | | | CGGTAATGAAAAACTGTTTT | | 4 | FF | |
| | | | TT | | | | |
| TCR_ | Alpha | TRAV21 | TGTGCTGTGGACACTGGAGG | 145 | TRAJ9 | CAVDTGGFKTIF | 71 |
| QVD_ | | | CTTCAAAACTATCTTT | | | | |
| 6 | Beta | TRBV6- | TGTGCCAGCACGTCAGGGGA | 146 | TRBJ1- | CASTSGDGNYGY | 72 |
| | | 5 | CGGAAACTATGGCTACACCT | | 2 | TF | |
| | | | TC | | | | |
| TCR_ | Alpha | TRAV21 | TGTGCTGTTAATACTGGAGG | 147 | TRAJ9 | CAVNTGGFKTIF | 73 |
| QVD_ | | | CTTCAAAACTATCTTT | | | | |
| 7 | Beta | TRBV27 | TGTGCCAGCACCTCGGGGGA | 148 | TRBJ1- | CASTSGDGNEKL | 74 |
| | | | TGGTAATGAAAAACTGTTTT | | 4 | FF | |
| | | | TT | | | | |
| TCR_ | Alpha | TRAV21 | TGTGCTGTGGATACTGGAGG | 149 | TRAJ9 | CAVDTGGFKTIF | 75 |
| QVD_ | | | CTTCAAAACTATCTTT | | | | |
| 8 | Beta | TRBV27 | TGTGCCAGCAGTTCAGGAGA | 150 | TRBJ1- | CASSSGDRDNYG | 76 |
| | | | CAGGGACAACTATGGCTACA | | 2 | YTF | |
| | | | CCTTC | | | | |
| TCR_ | Alpha | TRAV21 | TGTGCTGTCGATACTGGAGG | 151 | TRAJ9 | CAVDTGGFKTIF | 77 |
| QVD_ | | | CTTCAAAACTATCTTT | | | | |
| 9 | Beta | TRBV27 | TGTGCCAGCAGTGACCTTTC | 152 | TRBJ1- | CASSDLSGNQPQ | 78 |
| | | | AGGGAATCAGCCCCAGCATT | | 5 | HF | |
| | | | TT | | | | |
| TCR_ | Alpha | TRAV21 | TGTGCTGTGGACACTGGAGG | 153 | TRAJ9 | CAVDTGGFKTIF | 79 |
| QVD_ | | | CTTCAAAACTATCTTT | | | | |
| 10 | Beta | TRBV27 | TGTGCCAGCAGTTTAGATCT | 154 | TRBJ2- | CASSLDLSGANV | 80 |
| | | | GTCTGGGGCCAACGTCCTGA | | 6 | LTF | |
| | | | CTTTC | | | | |
| TCR_ | Alpha | TRAV21 | TGTGCTGTGGATACTGGAGG | 155 | TRAJ9 | CAVDTGGFKTIF | 81 |
| QVD_ | | | CTTCAAAACTATCTTT | | | | |
| 11 | Beta | TRBV27 | TGTGCCAGCAGTTCCCTGGA | 156 | TRBJ1- | CASSSLDGNTEA | 82 |
| | | | CGGCAACACTGAAGCTTTCT | | 1 | FF | |
| | | | TT | | | | |
| TCR_ | Alpha | TRAV21 | TGTGCTGTCGATACTGGAGG | 157 | TRAJ9 | CAVDTGGFKTIF | 83 |
| QVD_ | | | CTTCAAAACTATCTTT | | | | |
| 12 | Beta | TRBV20- | TGCAGTGCTAGAGAGGGCAG | 158 | TRBJ1- | CSAREGRVGNSP | 84 |
| | | 1 | GGTGGGGAATTCACCCCTCC | | 6 | LHF | |
| | | | ACTTT | | | | |
| TCR_ | Alpha | TRAV21 | TGTGCTGTGGATACTGGAGG | 159 | TRAJ9 | CAVDTGGFKTIF | 85 |
| QVD_ | | | CTTCAAAACTATCTTT | | | | |
| 13 | Beta | TRBV29- | TGCAGCGTTGGGGGACAGCT | 160 | TRBJ1- | CSVGGQLNQPQH | 86 |
| | | 1 | CAATCAGCCCCAGCATTTT | | 5 | F | |
| TCR_ | Alpha | TRAV21 | TGTGCTGTGAATACTGGAGG | 161 | TRAJ9 | CAVNTGGFKTIF | 87 |
| QVD_ | | | CTTCAAAACTATCTTT | | | | |
| 14 | Beta | TRBV27 | TGTGCCAGCAGTGGGACAGG | 162 | TRBJ1- | CASSGTGLSNEK | 88 |
| | | | GCTTAGTAATGAAAAACTGT | | 4 | LFF | |
| | | | TTTTT | | | | |
| TCR_ | Alpha | TRAV21 | TGTGCTCCAAATACTGGAGG | 163 | TRAJ9 | CAPNTGGFKTIF | 89 |
| QVD_ | | | CTTCAAAACTATCTTT | | | | |
| 15 | Beta | TRBV4- | TGCGCCAGCAGCCAAGAACC | 164 | TRBJ2- | CASSQEPSGGYE | 90 |
| | | 3 | TAGCGGGGGCTACGAGCAGT | | 7 | QYF | |
| | | | ACTTC | | | | |
| TCR_ | Alpha | TRAV21 | TGTGCTGTCAATACTGGAGG | 165 | TRAJ9 | CAVNTGGFKTIF | 91 |
| QVD_ | | | CTTCAAAACTATCTTT | | | | |
| 16 | Beta | TRBV7- | TGTGCCAGCAGCTTAAGGGT | 166 | TRBJ2- | CASSLRVYETQY | 92 |
| | | 7 | TTACGAGACCCAGTACTTC | | 5 | F | |
| TCR_ | Alpha | TRAV21 | TGTGCTGTCAATACTGGAGG | 167 | TRAJ9 | CAVNTGGFKTIF | 93 |
| QVD_ | | | CTTCAAAACTATCTTT | | | | |
| 17 | Beta | TRBV3- | TGTGCCAGCAGCCTAAGTTG | 168 | TRBJ1- | CASSLSWDRDRS | 94 |
| | | 1 | GGACAGGGACAGAAGCTACA | | 2 | YTF | |
| | | | CCTTC | | | | |
| TCR_ | Alpha | TRAV21 | TGTGCTGTTGATACTGGAGG | 169 | TRAJ9 | CAVDTGGFKTIF | 95 |
| QVD_ | | | CTTCAAAACTATCTTT | | | | |
| 18 | Beta | TRBV20- | TGCAGTGCGGAAGGGACTAG | 170 | TRBJ2- | CSAEGTSGKVGY | 96 |
| | | 1 | CGGGAAGGTAGGGTATGAGC | | 1 | EQFF | |
| | | | AGTTCTTC | | | | |
| TCR_ | Alpha | TRAV21 | TGTGCTGTCAATACTGGAGG | 171 | TRAJ9 | CAVNTGGFKTIF | 97 |
| QVD_ | | | CTTCAAAACTATCTTT | | | | |
| 19 | Beta | TRBV6- | TGTGCCAGCAGTCCCTCCCG | 172 | TRBJ2- | CASSPSRPRVEE | 98 |
| | | 5 | ACCCAGGGTCGAGGAGACCC | | 5 | TQYF | |
| | | | AGTACTTC | | | | |
| TCR_ | Alpha | TRAV21 | TGTGCTGTTAATACTGGAGG | 173 | TRAJ9 | CAVNTGGFKTIF | 99 |
| QVD_ | | | CTTCAAAACTATCTTT | | | | |
| 20 | Beta | TRBV27 | TGTGCCAGCAGTTCACAGGG | 174 | TRBJ1- | CASSSQGTLGVP | 100 |
| | | | GACCCTCGGGGTTCCCCTCC | | 6 | LHF | |
| | | | ACTTT | | | | |
| TCR_ | Alpha | TRAV21 | TGTGCTGTAAATACTGGAGG | 175 | TRAJ9 | CAVNTGGFKTIF | 101 |
| QVD_ | | | CTTCAAAACTATCTTT | | | | |
| 21 | Beta | TRBV27 | TGTGCCAGCAGTACGACAGG | 176 | TRBJ1- | CASSTTGLANEK | 102 |
| | | | GTTGGCTAATGAAAAACTGT | | 4 | LFF | |
| | | | TTTTT | | | | |
| TCR_ | Alpha | TRAV21 | TGTGCTGTTAATACTGGAGG | 177 | TRAJ9 | CAVNTGGFKTIF | 103 |
| QVD_ | | | CTTCAAAACTATCTTT | | | | |
| 22 | Beta | TRBV27 | TGTGCCAGCACCTCGGGGGA | 178 | TRBJ1- | CASTSGDGNEKL | 104 |
| | | | TGGTAATGAAAAACTGTTTT | | 4 | FF | |
| | | | TT | | | | |
| TCR_ | Alpha | TRAV21 | TGTGCTGTGAATACTGGAGG | 179 | TRAJ9 | CAVNTGGFKTIF | 105 |
| QVD_ | | | CTTCAAAACTATCTTT | | | | |
| 23 | Beta | TRBV5- | TGCGCCAGCAGCCCCGCATG | 180 | TRBJ2- | CASSPAWLDEQY | 106 |
| | | 1 | GTTGGACGAGCAGTACTTC | | 7 | F | |
| TCR_ | Alpha | TRAV21 | TGTGCTGTGGATACTGGAGG | 181 | TRAJ9 | CAVDTGGFKTIF | 107 |
| QVD_ | | | CTTCAAAACTATCTTT | | | | |
| 24 | Beta | TRBV27 | TGTGCCAGCAGTAGCGGGGA | 182 | TRBJ1- | CASSSGDRDNYG | 108 |
| | | | CAGGGATAACTATGGCTACA | | 2 | YTF | |
| | | | CCTTC | | | | |
| TCR_ | Alpha | TRAV21 | TGTGCTGTCGATACTGGAGG | 183 | TRAJ9 | CAVDTGGFKTIF | 109 |
| QVD_ | | | CTTCAAAACTATCTTT | | | | |
| 25 | Beta | TRBV6- | TGTGCCAGCAGTTACGACCG | 184 | TRBJ2- | CASSYDRYEQYF | 110 |
| | | 2 | CTACGAGCAGTACTTC | | 7 | | |
| TCR_ | Alpha | TRAV21 | TGTGCTGTGGATACTGGAGG | 185 | TRAJ9 | CAVDTGGFKTIF | 111 |
| QVD_ | | | CTTCAAAACTATCTTT | | | | |
| 26 | Beta | TRBV6- | TGTGCCAGCAGTTACGCATC | 186 | TRBJ1- | CASSYASGDYGY | 112 |
| | | 5 | TGGGGACTATGGCTACACCT | | 2 | TF | |
| | | | TC | | | | |
| TCR_ | Alpha | TRAV21 | TGTGCTGTGGATACTGGAGG | 187 | TRAJ9 | CAVDTGGFKTIF | 113 |
| QVD_ | | | CTTCAAAACTATCTTT | | | | |
| 27 | Beta | TRBV2 | TGTGCCAGCGCGCTCTATAG | 188 | TRBJ2- | CASALYSTDTQY | 114 |
| | | | CACAGATACGCAGTATTTT | | 3 | F | |
| TCR_ | Alpha | TRAV21 | TGTGCTGTCGATACTGGAGG | 189 | TRAJ9 | CAVDTGGFKTIF | 115 |
| QVD_ | | | CTTCAAAACTATCTTT | | | | |
| 28 | Beta | TRBV4- | TGCGCCAGCAGCCAAGAAGG | 190 | TRBJ2- | CASSQEGAPAGE | 116 |
| | | 1 | GGCCCCAGCCGGGGAGCTGT | | 2 | LFF | |
| | | | TTTTT | | | | |
| TCR_ | Alpha | TRAV21 | TGTGCTGTCGATACTGGAGG | 191 | TRAJ9 | CAVDTGGFKTIF | 117 |
| QVD_ | | | CTTCAAAACTATCTTT | | | | |
| 29 | Beta | TRBV27 | TGTGCCAGCAGCCCCGGGGA | 192 | TRBJ1- | CASSPGDRSYGY | 118 |
| | | | CAGGAGCTATGGCTACACCT | | 2 | TF | |
| | | | TC | | | | |
| TCR_ | Alpha | TRAV21 | TGTGCTGTCGATACTGGAGG | 193 | TRAJ9 | CAVDTGGFKTIF | 119 |
| QVD_ | | | CTTCAAAACTATCTTT | | | | |
| 30 | Beta | TRBV27 | TGTGCCAGCAGTCCCGCGGA | 194 | TRBJ1- | CASSPADRGDTI | 120 |
| | | | CAGGGGTGACACCATATATT | | 3 | YF | |
| | | | TT | | | | |
| TCR_ | Alpha | TRAV21 | TGTGCCGTAGATACTGGAGG | 195 | TRAJ9 | CAVDTGGFKTIF | 121 |
| QVD_ | | | CTTCAAAACTATCTTT | | | | |
| 31 | Beta | TRBV27 | TGTGCCAGCAGTTTATCGGA | 196 | TRBJ1- | CASSLSDGNYGY | 122 |
| | | | CGGAAACTATGGCTACACCT | | 2 | TF | |
| | | | TC | | | | |
| TCR_ | Alpha | TRAV21 | TGTGCTGTTGATACTGGAGG | 197 | TRAJ9 | CAVDTGGFKTIF | 123 |
| QVD_ | | | CTTCAAAACTATCTTT | | | | |
| 32 | Beta | TRBV6- | TGTGCCAGCAGTTTGCGTGG | 198 | TRBJ2- | CASSLRGLAIPY | 124 |
| | | 2 | ACTAGCGATCCCCTACGAGC | | 7 | EQYV | |
| | | | AGTACGTC | | | | |
| TCR_ | Alpha | TRAV21 | TGTGCTGTGGATACTGGAGG | 199 | TRAJ9 | CAVDTGGFKTIF | 125 |
| QVD_ | | | CTTCAAAACTATCTTT | | | | |
| 33 | Beta | TRBV27 | TGTGCCAGCAGTTTAGGGGA | 200 | TRBJ1- | CASSLGDGNYGY | 126 |
| | | | CGGGAACTATGGCTACACCT | | 2 | TF | |
| | | | TC | | | | |
| TCR_ | Alpha | TRAV21 | TGTGCTGTAGATACTGGAGG | 201 | TRAJ9 | CAVDTGGFKTIF | 127 |
| QVD_ | | | CTTCAAAACTATCTTT | | | | |
| 34 | Beta | TRBV27 | TGTGCCAGCAGTTTAGCAGA | 202 | TRBJ1- | CASSLADGNYGY | 128 |
| | | | CGGAAACTATGGCTACACCT | | 2 | TF | |
| | | | TC | | | | |
| TCR_ | Alpha | TRAV21 | TGTGCTGTGGATACTGGAGG | 203 | TRAJ9 | CAVDTGGFKTIF | 129 |
| QVD_ | | | CTTCAAAACTATCTTT | | | | |
| 35 | Beta | TRBV27 | TGTGCCAGCAGTTTGGCAGA | 204 | TRBJ1- | CASSLADGNEKL | 130 |
| | | | CGGTAATGAAAAACTGTTTT | | 4 | FF | |
| | | | TT | | | | |
| TCR_ | Alpha | TRAV21 | TGTGCTGTTGATACTGGAGG | 205 | TRAJ9 | CAVDTGGFKTIF | 131 |
| QVD_ | | | CTTCAAAACTATCTTT | | | | |
| 36 | Beta | TRBV27 | TGTGCCAGCAGTTTCGATCT | 206 | TRBJ2- | CASSFDLSGANV | 132 |
| | | | CTCTGGGGCCAACGTCCTGA | | 6 | LTF | |
| | | | CTTTC | | | | |
| TCR_ | Alpha | TRAV21 | TGTGCTGTGAATACTGGAGG | 207 | TRAJ9 | CAVNTGGFKTIF | 133 |
| QVD_ | | | CTTCAAAACTATCTTT | | | | |
| 37 | Beta | TRBV20- | TGCAGTGCTAGAGACCGGCA | 208 | TRBJ1- | CSARDRQGDEKL | 134 |
| | | 1 | GGGGGATGAAAAACTGTTTT | | 4 | FF | |
| | | | TT | | | | |

Table 1 and Table 2 provide information of TCRs that bind YSKNKVWEV (SEQ ID NO: 1) presented by HLA-B*08:01 and QVDYYGLYY (SEQ ID NO: 59) presented by HLA-A*01:01, respectively. Each table identifies the CDR3 amino acid sequence, V gene, J gene, and the nucleotide sequence of the junction area that encodes the CDR3, with respect to each alpha or beta chain variable domain. Such information can be used to generate complete alpha or beta chain variable domain sequences by taking the following steps:
1. Identify the nucleotide sequence of the V gene according to public databases such as IMGT or Genbank.
2. Identify the nucleotide sequence of the corresponding J gene (in the same row of the table) from the database.
3. Align the V and J gene nucleotide sequences with the corresponding junction nucleotide sequence (in the same row of the table), such that the 3′ end of the V gene overlaps with the 5′ end of the junction, and the 3′ end of the junction overlaps with the 5′ end of the J gene.
4. Obtain a complete nucleotide sequence, from the 5′ end of the V gene to the 3′ end of the J gene, according to the alignment. This is the nucleotide sequence of the variable domain.
5. Translate the nucleotide sequence into an amino acid sequence, thereby to obtain the amino acid sequence of the variable domain.

It is understood that some V and J genes have multiple alleles in the human population. In certain embodiments, the most dominant allele in the human population is contemplated herein. In certain embodiments, additional alleles in the human population are also contemplated herein.

Accordingly, complete amino acid sequences of a and β chain variable domains of each of the TCRs set forth in Table 1 and Table 2 can be determined based on the information in these tables. Further, the CDR1 and CDR2 sequences of each α or β chain variable domain can be determined under IMGT, Kabat, or Chothia definition (reviewed in Lefranc Front Immunol. (2014); 5: 22; see also Kabat et al., U.S. Department of Health and Human Services (USDHHS), National Institute of Health (NIH) Publication (1991). p. 91-3242; Chothia et al., Mol. Biol. (1987) 196(4):901-17).

It has been found, in certain instances, that CDR1a can interact with the N-terminal part of the T cell epitope and CDR1β can interact with the C-terminal part of the antigen. Accordingly, in certain embodiments, the TCR disclosed herein further comprises the corresponding CDR1α and CDR1β amino acid sequences according to the information provided in Table 1 or Table 2. In certain embodiments, the TCR comprises the corresponding CDR1α, CDR2α, CDR3α, CDR1β, CDR2β, and CDR3β amino acid sequences according to the information provided in Table 1 or Table 2. In certain embodiments, the TCR comprises Vα and Vβ amino acid sequences at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the corresponding Vα and Vβ sequences, respectively, according to the information provided in Table 1 or Table 2. In certain embodiments, a TCR disclosed herein is derived from a TCR selected from TCR_YSK_1 to TCR_YSK_14 and TCR_QVD_1 to TCR_QVD_37.

Also contemplated herein are affinity matured TCRs, comprising one or more amino acid mutations (e.g., substitutions, insertions, and/or deletions) relative to a TCR disclosed herein, for increasing the affinity and/or specificity of the TCR for its epitope presented by a cognate MHC. Affinity maturation methods for TCRs are known in the art, for example in vitro, directed evolution has been used to generate TCRs with higher affinity for a specific peptide MHC. The three different display methods that have been used are yeast display (Holler et al., Nat Immunol, (2003) 4, 55-62; Holler et al., Proc Natl Acad Sci USA, (2000) 97, 5387-92), phage display (Li et al., Nat Biotechnol, (2005) 23, 349-54), and T cell display (Chervin et al., J Immunol Methods, (2008) 339, 175-84). In all three approaches, the process involves engineering, or modifying, a TCR that exhibits a low affinity of the wild-type TCR, so that affinity of mutants of the TCR have increased affinity for the cognate peptide MHC (the original antigen that the T cells were specific for). Thus, the wild-type TCR can be used as a template for producing mutagenized libraries in one or more of the CDRs, and mutants with higher affinity are selected by binding to the cognate peptide-MHH.

In certain embodiments, the TCR binds the corresponding epitope-MHC complex with a K_{D} of 1 μM or lower, 500 nM or lower, 400 nM or lower, 300 nM or lower, 200 nM or lower, 175 nM or lower, 150 nM or lower, 125 nM or lower, 100 nM or lower, 75 nM or lower, 50 nM or lower, 25 nM or lower, or 10 nM or lower. In certain embodiments, the TCR comprises a Vα and a Vβ that, when incorporated into a full-length TCR, allows the full-length TCR to be activated by a corresponding T cell epitope, namely, of YSKNKVWEV (SEQ ID NO: 1) presented by HLA-B*08:01 or QVDYYGLYY (SEQ ID NO: 59) presented by HLA-A*01:01. TCR binding affinity can be assessed using a number of methods known in the art. For example, in a surface plasmon resonance (SPR) assay, soluble TCR can be immobilized to the surface of a sensor chip, and the association and dissociation of a soluble μMHC complex can be measured (see, e.g., Jones et al., Biochemistry (2008) 47(47):12398-12408). In a cell-based assay, fluorescence microscopy can measure the TCR-μMHC affinity using a soluble μMHC bound to T cells that express a full-length TCR (see, e.g., Nauerth et al., Sci. Transl. Med. (2013) 5(192):192ra87). TCR activation can also be assessed using methods known in the art. For example, target cells expressing a corresponding MHC and presenting a corresponding T cell epitope (e.g., expressing a protein comprising the epitope) can be contacted with T cells genetically engineered to express the TCR. Proliferation of the engineered T cells can be assessed by carboxyfluorescein succinimidyl ester (CFSE) dilution assay, Ki-67 staining, tritiated thymidine incorporation, BrdU incorporation, or quantitation of ATP levels. Activation of the engineered T cells can be assessed by staining for cell surface markers (e.g., upregulation of CD69, CD27, CD137, CD154, CD25, CD44, or CD107a, or downregulation of CD62L or CCR7) or cytokines (e.g., IFNγ or TNFα) or detecting secretion of cytokine proteins (e.g., IFNγ or TNFα).

In certain embodiments, the TCR disclosed herein further comprises one or more regions present in a naturally occurring TCR or variants thereof. In a full-length TCR (e.g., a naturally occurring full-length TCR), the α-chain typically comprises, from the N-terminus to the C-terminus, a Vα, an α-chain constant region (Cα), an α-chain hinge region (a.k.a., a connecting peptide), an α-chain transmembrane region, and an α-chain intracellular region (a.k.a. a cytoplasmic tail); the β-chain typically comprises, from the N-terminus to the C-terminus, a Vβ, a β-chain constant region (CO), a β-chain hinge region (a.k.a., a connecting peptide), a β-chain transmembrane region, and a β-chain intracellular region (a.k.a. a cytoplasmic tail).

In certain embodiments, a TCR disclosed herein further comprises a Cα and/or a Cβ. In certain embodiments, the TCR further comprises an α-chain hinge region and/or a β-chain hinge region. The Cα and/or Cβ can comprise amino acid mutations to stabilize the interaction between the Cα and the C₃ (see, e.g., Boulter et al., (2003) Protein Engineering, Design and Selection 16(9):707-11; WO2019046778A1). In certain embodiments, the α-chain hinge region and the β-chain hinge region each comprises a Cys residue that form an inter-chain disulfide bond, which may stabilize the TCR. In certain embodiments, the TCR further comprises one or more transmembrane regions, for example, an α-chain transmembrane region and/or a β-chain transmembrane region. In certain embodiments, the TCR further comprises one or more intracellular regions, for example, an α-chain intracellular region and/or a β-chain intracellular region. In certain embodiments, the TCR is a full-length TCR.

### III. Engineered TCRs

The present disclosure also provides engineered TCRs, which include but are not limited to chimeric T-cell receptor, soluble TCR, multispecific (e.g., bispecific) immune cell (e.g., T cell or NK cell) engager, and TCR mimetic (see Chandran and Klebanoff (2019) Immunol. Rev. 290:127-47; Goebeler and Bargou (2020) Nat. Rev. Clin. Oncol. 17:418-34). The modifications made in engineered TCRs are described in two subsections below, “engineered transmembrane TCRs” and “engineered soluble TCRs,” based on the context that the modifications primarily pertain to. It is understood, however, that a modification described in one subsection may be applicable to the other, where appropriate under the circumstances. In certain embodiments, the TCR is isolated, non-naturally occurring, and/or engineered.

### Engineered Transmembrane TCRs

In certain embodiments, an engineered transmembrane TCR disclosed herein is designed to reduce mispairing of the recombinant TCR αβ chains with endogenous TCR αβ chains, thereby to reduce safety and efficacy concerns. For example, mispairing of endogenous TCR chains can be reduced or prevented by swapping the constant domains between the TCR α and β chains of a therapeutic TCR. When paired, domain-swapped (ds)TCRs assemble with CD3, express on the cell surface, and mediate antigen-specific T cell responses. By contrast, dsTCR chains mispaired with endogenous TCR chains cannot properly assemble with CD3 or signal.

In certain embodiments, the engineered TCR comprises one or more transmembrane domains different from the wild-type TCR transmembrane domains. In certain embodiments, the α chain transmembrane domain is modified by introducing one or more hydrophobic amino acid substitutions (see, e.g., Haga-Friedman et al., J. Immunol (2012) 188(11):5538-46). In certain embodiments, the engineered TCR comprises one or more transmembrane domains derived from a natural source or a synthetic source. Where the source is natural, the transmembrane domain can be derived from any membrane-bound or transmembrane protein, for example, the transmembrane domains of the CD3-zeta, CD28, CD3-epsilon, CD45, CD4, CD5, CD8, CD9, CD16, CD22, CD33, CD37, CD64, CD80, CD86, CD134, CD137/(4-1BB), and/or CD154. Where the source is synthetic, the transmembrane domain can comprise predominantly hydrophobic residues such as leucine and valine. In certain embodiments, the synthetic transmembrane domain comprises a triplet of phenylalanine, tryptophan and valine at each terminus.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the receptor includes one or both of such signaling components. In certain embodiments, the engineered TCR comprises one or more intracellular signaling domains different from the wild-type TCR intracellular domains.

In some aspects, the engineered TCR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CD8, CD22, CD79a, CD79b, and CD66d. In some embodiments, cytoplasmic signaling molecule(s) in the engineered TCR contain(s) a cytoplasmic signaling domain, portion thereof, or sequence derived from CD3 zeta.

In the context of a natural TCR, full activation generally requires not only signaling through the TCR, but also a costimulatory signal. Thus, in some embodiments, to promote full activation, a component for generating secondary or co-stimulatory signal is also included in the receptor. In other embodiments, the receptor does not include a component for generating a costimulatory signal. In some aspects, an additional receptor is expressed in the same cell and provides the component for generating the secondary or costimulatory signal.

In some embodiments, the engineered TCR encompasses one or more, e.g., two or more, costimulatory domains and/or an activation domain, e.g., primary activation domain, in the cytoplasmic portion. In some embodiments, the engineered TCR includes the intracellular signaling domain and/or transmembrane portion of a costimulatory receptor, such as CD28, 4-1BB, OX40, DAP10, or ICOS. In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domain, linked to a CD3 zeta intracellular domain. In some aspects, the same engineered TCR includes both the activating and costimulatory components.

In some embodiments, the cell signaling domains of the engineered TCR include a CD3 transmembrane domain, a CD3 intracellular signaling domain, and/or other CD transmembrane domains. In some embodiments, the engineered TCR further includes a portion of one or more additional molecules such as Fc receptor-gamma, CD8, CD4, CD25, or CD16. For example, in some aspects, the engineered TCR includes a chimeric molecule between CD3-zeta or Fc receptor-gamma and CD8, CD4, CD25 or CD16.

In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the receptor.

In certain embodiments, the engineered TCR comprises the Vα and Vβ domains disclosed herein and the transmembrane and intracellular domain of another protein (e.g., another protein in the TCR/CD3 complex). In certain embodiments, the Vα and Vβ domains are linked to a complete human CD3-zeta protein (see, e.g., J. Immunol Jun. 1, 2008, 180 (11) 7736-7746; Gene Ther. 2000 August; 7(16):1369-77; and The Open Gene Therapy Journal, 2011, 4: 11-22).

Recombinant TCRs are typically glycosylated when expressed in T cells. In certain embodiments, the glycosylation (e.g., N-glycosylation) pattern of the engineered TCR is modified by mutagenesis such that one or more of the N-glycosylation sites of a TCR, such as constant region glycosylation sites, are removed. Such mutations include, for example, a change in the glycosylation site NXS/T to QXS/T in the constant region of the α- and/or β-chains. The TCR could possess the NXS/T to QXS/T mutation in one of the α- or β-chains or in both chains. In a TCR comprising a murine constant region, the glycosylation site NQT in the murine constant region can be modified to QQT.

### Engineered Soluble TCRs

In certain embodiments, the engineered TCR is a soluble TCR. Such soluble TCRs may be engineered by removing at least the intracellular and transmembrane regions of a full-length TCR (see, e.g., U.S. Pat. No. 8,519,100, which describes the synthesis of soluble T-cell receptors). In certain embodiments, a soluble TCR comprises an engineered TCRα and an engineered TCRβ chain. In certain embodiments, a soluble TCR comprises a Vα and a Vβ. In certain embodiments, a soluble TCR comprises a Vα and a Vβ. In certain embodiments, a soluble TCR further comprises an alpha chain constant domain (Cα) and a beta chain constant domain (Cβ). In certain embodiments, a soluble TCR comprises a Vα, a Cα, a Vβ, and a Cβ, optionally wherein the Vα is linked to the N-terminus of the Cα and the Vβ is linked to the N-terminus of the Cβ. In certain embodiments, a soluble TCR comprises the extracellular portion of a full-length TCR, including variable domain, constant region, and hinge region of each of the TCRα-chain and the TCRβ-chain, optionally linked in the orientation as in a naturally occurring TCR. In certain embodiments, the α-chain domains/regions and the β-chain domains/regions are present in two separate polypeptides. In certain embodiments, the α-chain domains/regions and the β-chain domains/regions are linked into a single polypeptide, thereby to form a single chain TCR (scTCR).

In certain embodiments, the TCR (e.g., soluble TCR) can comprise one or more mutations that stabilize the interaction between the engineered TCRα and an engineered TCRβ chain. The soluble TCR can comprise one or more mutations that stabilize the interaction between the Cα and the Cβ. In certain embodiments, the mutation can comprise one or more substitutions to facilitate heterodimerization of an α-chain domains/regions and a β-chain domains/regions. For example, cysteine substitutions can be introduced to enable formation of an inter-chain disulfide bond, thereby to stabilize the dimer. Cysteine substitutions can be located for example in the Vα, Cα, Vβ, and/or Cβ domain. In certain embodiments, a human TCR (e.g., soluble human TCR) comprises a Cys substitution at position 48 of Cα and a Cys substitution at position 57 of Cβ(see Cohen et al., Cancer Res. (2007) 67(8):3898-3903; Kuball et al., Blood (2007) 109(6):2331-38, and WO 2003/020763).

In certain embodiments, the Cα comprises a cysteine residue at position 48, and the Cβ comprises a cysteine residue at position 57. The cysteine residue at position 48 in the Cα and the cysteine residue at position 57 in the Cβ can form a disulfide bond (see Boulter et al., Protein Engineering, Design and Selection, (2003)16: 9, 707-711). In certain embodiments, the Cα comprises a phenylalanine residue at position 21, an isoleucine residue at position 32, and/or a threonine residue at position 72; and/or the Cβ comprises a lysine residue at position 18, an arginine residue at position 23, a proline residue at position 39, and/or an aspartic acid or glutamic acid at position 54 (see WO 2019/046778). In certain embodiments, the Cα comprises a phenylalanine residue at position 21, an isoleucine residue at position 32, and a threonine residue at position 72; and the Cβ comprises a lysine residue at position 18, an arginine residue at position 23, a proline residue at position 39, and an aspartic acid or glutamic acid at position 54. Alternatively or additionally, substitutions by charged amino acid residues promote the formation of a salt bridge between an α-chain domains/regions and a β-chain domains/regions. In certain embodiments, a human TCR (e.g., soluble human TCR) comprises an Asp substitution at position 210 of Cα and a Lys substitution at position 134 of Cβ (see Bialer et al., J. Immunol. (2010) 184(11):6232-41).

As used herein, for the purpose of amino acid numbering, the Cα reference sequence is human TRAC amino acid sequence

(SEQ ID NO: 209)
DIQNPDPAVYQLRDSKSSDKSVCLFTDFDSQTNVSQSKDSDVYITD
KTVLDMRSMDFKSNSAVAWSNKSDFACANAFNNSIIPEDTFFPSPE
SSCDVKLVEKSFETDTNLNFQNLSVIGFRILLLKVAGFNLLMTLRL
WSS.
The Cβ reference sequence is human
TRBC1 amino acid sequence
(SEQ ID NO: 210)
EDLNKVFPPEVAVFEPSEAEISHTQKATLVCLATGFFPDHVELSWW
VNGKEVHSGVSTDPQPLKEQPALNDSRYCLSSRLRVSATFWQNPRN
HFRCQVQFYGLSENDEWTQDRAKPVTQIVSAEAWGRADCGFTSVSY
QQGVLSATILYEILLGKATLYAVLVSALVLMAMVKRKDF
or
human TRBC2 amino acid sequence
(SEQ ID NO: 211)
EDLKNVFPPKVAVFEPSEAEISHTQKATLVCLATGFYPDHVELSWW
VNGKEVHSGVSTDPQPLKEQPALNDSRYCLSSRLRVSATFWQNPRN
HFRCQVQFYGLSENDEWTQDRAKPVTQIVSAEAWGRADCGFTSESY
QQGVLSATILYEILLGKATLYAVLVSALVLMAMVKRKDSRG.

The TCR alpha and beta chain may each comprise a dimerization domain, e.g., a heterologous dimerization domain. Exemplary dimerization domains include but are not limited to leucine zippers (see, e.g., Chang et al., Proc. Natl. Acad. Sci. USA. (1994) 91(24):11408-12), SH3 domains, jun/fos coiled-coil domains (Willcox et al., Immunity (1999a), 10, 357-365) and IgG Fc domains. In certain embodiments, the dimerization domains facilitate formation of a heterodimer. In certain embodiments, the dimerization domains are linked to the C-terminus of the Vα and the Vβ.

In certain embodiments, the soluble TCR is a single TCR (scTCR), optionally where the α-chain is linked to the β-chain via an amino acid linker. Exemplary scTCRs are described in U.S. Pat. No. 7,569,664, Willemsen et al., Gene Therapy (2000) 7:1369-77, and Richman et al., Mol Immunol. (2009) 46(5): 902-916. A scTCR may comprise a Vα fused to the N terminus of an α-chain constant region and hinge region, a Vβ fused to the N terminus of a β-chain constant region and hinge region, and a linker sequence linking the C-terminus of the α segment to the N-terminus of the β segment, or linking the N-terminus of the α segment to the C-terminus of the β segment. Exemplary single chain formats include but are not limited to αβ TCR polypeptides of the Vα-L-Vβ, Vα-Cα-L-Vβ, Vα-L-Vβ-Cβ, Vα-Cα-L-Vβ-Cβ, Vβ-L-Vα, Vβ-Cβ-L-Vα, or Vβ-Cβ-L-Vα-Cα format, wherein Vα and Vβ are TCR α and R variable regions respectively, Cα and Co are TCR α and 1 constant regions respectively, and L is a linker sequence. In certain embodiments, the hinge regions of the α and B segments of the scTCR are linked by a disulfide bond. In certain embodiments, the length of the linker sequence and the position of the disulfide bond are disposed such that the Vα and Vβ domains are mutually orientated substantially as in a native αβ TCR. In certain embodiments, in a scTCR, the Vα and the Vβ are covalently linked by a short peptide linker, for example, a serine-rich or glycine-rich linker such as (Gly₄Ser)₃.

### Soluble TCR Conjugates and Fusion Proteins

The soluble TCRs disclosed herein can be used as a targeting moiety for cells that present a cognate T cell epitope by a cognate MHC. It can be conjugated or fused with an effector moiety, including but not limited to binding domains for immune cell receptors, cytokines, chemotherapeutic agents, cytotoxic agents, detectable labels, and combinations thereof. The conjugation can be covalent or non-covalent. As used herein, a “TCR fusion protein” incorporated such conjugates, whether the soluble TCR is linked to the effector moiety by a traditional peptide bond or not.

In certain embodiments, a TCR fusion protein (for example a TCR conjugate) comprises a soluble TCR and a binding domain that binds a receptor on an outer surface of an immune cell, a cytotoxic agent, a detectable label, or a combination thereof. In certain embodiments, the binding domain binds a receptor on an outer surface of a T cell. In certain embodiments, the conjugation can be covalent or non-covalent.

In certain embodiments, the binding domain comprises a ligand of the immune cell receptor or an antibody, or a functional fragment or variant thereof that specifically binds the immune cell receptor. Antibody fragments and variants/analogues which are suitable for use in the compositions and methods described herein include minibodies, Fab fragments, F(ab′)₂ fragments, dsFv and scFv fragments, Nanobodies™ (comprise synthetic single immunoglobulin variable heavy domains derived from a camelid (e.g., camel or llama) antibody) and Domain Antibodies (comprise an affinity matured single immunoglobulin variable heavy domain or immunoglobulin variable light domain). Also contemplated are alternative protein scaffolds that exhibit antibody-like binding characteristics such as Affibodies (comprising engineered protein A scaffold), DARPins (Designed Ankyrin Repeat Proteins), and Anticalins, including engineered variants thereof, that can be used as binding domains for immune cell receptors. It is understood that the soluble TCR conjugate or soluble TCR fusion protein disclosed herein can comprise a plurality of binding domains, e.g., a plurality of binding domains for one or more immune cell receptors, thereby to generate a multispecific binding protein.

In certain embodiments, the binding domain comprises an antibody, or an antigen-binding fragment thereof, that binds the receptor on an outer surface of an immune cell. In some embodiments, the binding domain comprises a heavy chain variable domain (VH) and a light chain variable domain (VL) of an antibody. In certain embodiments, the binding domain takes the format of a single-chain variable fragment (scFv) or a Fab fragment.

In certain embodiments, the soluble TCR is fused (for example conjugated) with a binding domain for an immune cell receptor to form a TCR fusion protein, such as a multispecific (e.g., bispecific) immune cell engager. Exemplary immune cells include T cells (e.g., CD8+ T cells) and NK cells. Such multispecific immune cell engager can be designed to engage the immune cells to target cells recognized by the TCR (e.g., cancer cells), thereby to enhance the reactivity and cytotoxicity of the immune cell towards the target cell in vitro or in vivo. This approach allows the TCR variable domains to be utilized without the need of autologous and/or allogeneic cell transduction.

Exemplary receptors on T cells (e.g., effector T cells) that can be targeted by the multispecific immune cell engager include but are not limited to CD3, CD2, CD28, and CD8. In some embodiments, the binding domain is an anti-CD3 antibody, an anti-CD2-antibody, an anti-CD28 antibody, or an anti-CD8 antibody or an antigen-binding fragment thereof. In certain embodiments, the binding domain comprises a heavy chain variable domain (VH) and a light chain variable domain (VL) of an anti-CD3 antibody. Examples of anti-CD3 antibodies include but are not limited to OKT3, SP34, UCHT-1, BMA-031, and 12F6. For example, the murine IgG clone SP34 (EMBO J. 1985. 4(2):337-344; J. Immunol. 1986, 137(4):1097-100; J. Exp. Med. 1991, 174:319-326; J. Immunol. 1991, 147(9):3047-52) binds to human and cynomolgus CD3F. Exemplary sequences of anti-CD3 antibodies or derived scFvs are listed in Table 3. In certain embodiments, the VH comprises CDR1, CDR2, and CDR3 amino acid sequences set forth in SEQ ID NOs: 218, 219, and 220, respectively, and the VL comprises CDR1, CDR2, and CDR3 amino acid sequences set forth in SEQ ID NOs: 215, 216, and 217, respectively. In certain embodiments, the VH comprises an amino acid sequence at least 90% identical to SEQ ID NO: 213, and the VL comprises an amino acid sequence at least 90% identical to SEQ ID NO: 214. The CDR sequences in Table 3 are identified under the Kabat definition.

TABLE 3
Exemplary sequences of anti-CD3 antibodies

| SEQ | | |
|---|---|---|
| ID | | |
| NO: | Antibody | Amino Acid Sequence |
| 212 | UCHT1 scFv | AIQMTQSPSSLSASVGDRVTITCRASQDIR |
| | (linker | NYLNWYQQKPGKAPKLLIYYTSRLESGVPS |
| | sequence | RFSGSGSGTDYTLTISSLQPEDFATYYCQQ |
| | underlined) | GNTLPWTFGQGTKVEIKGGGGSGGGGSGGG |
| | | GSGGGGSGGGSEVQLVESGGGLVQPGGSLR |
| | | LSCAASGYSFTGYTMNWVRQAPGKGLEWVA |
| | | LINPYKGVSTYNQKFKDRFTISVDKSKNTA |
| | | YLQMNSLRAEDTAVYYCARSGYYGDSDWYF |
| | | DVWGQGTLVTVSS |
| 213 | UCHT1 VL | AIQMTQSPSSLSASVGDRVTITCRASQDIR |
| | | NYLNWYQQKPGKAPKLLIYYTSRLESGVPS |
| | | RFSGSGSGTDYTLTISSLQPEDFATYYCQQ |
| | | GNTLPWTFGQGTKVEIK |
| 214 | UCHT1 VH | EVQLVESGGGLVQPGGSLRLSCAASGYSFT |
| | | GYTMNWVRQAPGKGLEWVALINPYKGVSTY |
| | | NQKFKDRFTISVDKSKNTAYLQMNSLRAED |
| | | TAVYYCARSGYYGDSDWYFDVWGQGTLVTV |
| | | SS |
| 215 | UCHT1 CDR1 | RASQDIRNYLN |
| | VL | |
| 216 | UCHT1 CDR2 | YTSRLES |
| | VL | |
| 217 | UCHT1 CDR3 | QQGNTLPWT |
| | VL | |
| 218 | UCHT1 CDR1 | GYTMN |
| | VH | |
| 219 | UCHT1 CDR2 | LINPYKGVSTYNQKFKD |
| | VH | |
| 220 | UCHT1 CDR3 | SGYYGDSDWYFDV |
| | VH | |
| 221 | SP34 VL | QAVVTQESALTTSPGETVTLTCRSSTGAVT |
| | | TSNYANWVQEKPDHLFTGLIGGTNFRAPGV |
| | | PARFSGSLIGDKAALTITGAQTEDEAIYFC |
| | | ALWYSNLWVFGGGTKLTVL |
| 222 | SP34 VH | VQLVESGGGLVQPKGSLKLSCAASGFTFNT |
| | | YAMNWVRQAPGKGLEWVARIRSKYNNYATY |
| | | YADSVKDRFTISRDDSQSILYLQMNNLKTE |
| | | DTAMYYCVRHGNFGNSYVSWFAYWGQGTLV |
| | | TVSA |
| 223 | SP34 CDR1 | RSSTGAVTTSNYAN |
| | VL | |
| 224 | SP34 CDR2 | GTNFRAP |
| | VL | |
| 225 | SP34 CDR3 | ALWYSNLWV |
| | VL | |
| 226 | SP34 CDR1 | TYAMN |
| | VH | |
| 227 | SP34 CDR2 | RIRSKYNNYATYYADSVKD |
| | VH | |
| 228 | SP34 CDR3 | HGNFGNSYVSWFAY |
| | VH | |
| 229 | OKT3 VL | QIVLTQSPAIMSASPGEKVTMTCSASSSVS |
| | | YMNWYQQKSGTSPKRWIYDTSKLASGVPAH |
| | | FRGSGSGTSYSLTISGMEAEDAATYYCQQW |
| | | SSNPFTFGSGTKLEINR |
| 230 | OKT3 VH | QVQLQQSGAELARPGASVKMSCKASGYTFT |
| | | RYTMHWVKQRPQGLEWIGYINPSRGYTNTN |
| | | QKFKDKATLTTDKSSSTAYMQLSSLTSEDS |
| | | AVYYCARYYDDHYCLDYWGQGTTLTVSS |
| 231 | OKT3 CDR1 | SASSSVSYMN |
| | VL | |
| 232 | OKT3 CDR2 | DTSKLAS |
| | VL | |
| 233 | OKT3 CDR3 | QQWSSNPFT |
| | VL | |
| 234 | OKT3 CDR1 | RYTMH |
| | VH | |
| 235 | OKT3 CDR2 | YINPSRGYTNTNQKFKD |
| | VH | |
| 236 | OKT3 CDR3 | YYDDHYCLDY |
| | VH | |

In certain embodiments, the soluble TCR variable domains (e.g., scTCR) is linked to the binding domain for the immune cell receptor (e.g., a scFv) via a peptide linker, thereby to form a fusion protein (e.g., single-chain fusion protein). In certain embodiments, the scFv is linked to the N-terminus of a TCR Vβ.

Exemplary receptors on NK cells that can be targeted by the multispecific immune cell engager include but are not limited to CD16, CD16a, NKp30, NKp46, and NKG2D.

In some embodiments, the binding domain is an anti-CD16 antibody, an anti-CD16α-antibody, an anti-NKp30 antibody, an anti-CD NKp46 antibody, or an anti-NKG2D antibody or an antigen-binding fragment thereof. In certain embodiments, the binding domain comprises a heavy chain variable domain (VH) and a light chain variable domain (VL) of an anti-NKG2D antibody. In some embodiments, the binding domain is a natural ligand of CD16, CD16a, NKp30, NKp46, or NKG2D. For example, a CD16 binding domain can comprise a Fc domain or a fragment thereof comprising the hinge and CH2 domain.

In certain embodiments, the soluble TCR is linked or fused to the N- or C-terminus of the binding domain (BD). Exemplary formats C-terminal fusion include but are not limited to αβ TCR polypeptides of the comprising formats Vα-L1-Vβ-BD, Vα-Cα-L1-Vβ-BD, Vα-L1-Vβ-Cβ-BD, Vβ-L1-Vα-BD, Vα-Cα-L1-Vβ-Cβ-BD, Vβ-L1-Vα-BD, V3-Cβ-L1-Vα-BD, V3-Cβ-L1-Vα-Cα-BD or Vα-L1-Vβ-L2-BD, Vα-Cα-L1-Vβ-L2-BD, Vα-L1-Vβ-Cβ-L2-BD, VP-L1-Vα-L2-BD, Vα-Cα-L1-Vβ-Cβ-L2-BD, Vβ-L1-Vα-L2-BD, V3-Cβ-L1-Vα-L2-BD, Vβ-Cβ-L1-Vα-Cα-L2-BD, wherein Vα and Vβ are TCR α and R variable regions respectively, Cα and Cβ are TCR α and R constant regions respectively, BD is binding domain, and L1 and L2 are linker sequences. Exemplary formats for N-terminal fusion include but are not limited to αβ TCR polypeptides of the comprising formats BD-Vα-L1-Vβ, BD-Vα-Cα-L1-Vβ, BD-Vα-L1-V -Cβ, BD-Vβ-L1-Vα, BD-Vα-Cα-L1-Vβ-C, BD-Vβ-L1-Vα, BD-Vβ-Cβ-L1-Vα, BD-Vβ-Cβ-L1-Vα-Cα or BD-L2-Vα-L1-Vβ, BD-L2-Vα-Cα-L1-Vβ, BD-L2-Vα-L1-V -Cβ, BD-L2-Vβ-L1-Vα, BD-L2-Vα-Cα-L1-Vβ-Cβ, BD-L2-Vβ-L1-Vα, BD-L2-Vβ-Cβ-L1-Vα, BD-L2-Vβ-Cβ-L1-Vα-Cα.

In certain embodiments, the soluble TCR fusion protein comprises additional domains that improve therapeutic efficacy. Additional domains comprise for example IgG-derived formats (for example derived from full length or fragments of IgG1, IgG2, IgG3 and IgG4 chains). In certain embodiments, the additional domain comprises a fragment crystallizable (Fc) region derived from an IgG. Fc regions comprise two or three heavy chain constant domains (CH domains CH2, CH3, and CH1) that can be used as additional domains. In certain embodiments, the soluble TCR fusion protein has a symmetric or asymmetric design. In certain embodiments, the soluble TCR fusion protein can comprise, one, two, three, or more chains that form a stable soluble complex with each other.

In certain embodiments, the soluble TCR fusion protein comprises two polypeptide chains, for example a first polypeptide chain comprising, from N-terminus to C-terminus, the Vβ of the TCR, a Cβ domain of the TCR, an optional linker, and a single chain variable fragment (scFv) comprising a VH and a VL of the antibody that binds the receptor on an outer surface of an immune cell (Vβ-Cβ—VH-VL or Vβ-Cβ-L-VH-VL), and a second polypeptide chain comprising, from N-terminus to C-terminus, the Vα of the TCR, and a Cα domain of the TCR (Vα-Cα) (see U.S. Pat. No. 8,519,100). In certain embodiments, the two polypeptides form a symmetric dimeric complex by pairing of the Vβ, Vα and the Cβ Cα regions of the TCR. In certain embodiments, the soluble TCR fusion protein comprises (a) a first polypeptide chain comprising, from N-terminus to C-terminus, a first variable domain of an antibody, an optional first linker, a first variable domain of the TCR, and a first IgG Fc region comprising hinge, CH2, and CH3 domains; and (b) a second polypeptide chain comprising, from N-terminus to C-terminus, a second variable domain of the TCR, an optional second linker, a second variable domain of the antibody, and a second IgG Fc region comprising hinge, CH2, and CH3 domains, wherein the first and second IgG Fc regions pair to form a dimer (see U.S. Patent Application Publication No.: US20190016801A1). In certain embodiments, the antibody binds the receptor on an outer surface of an immune cell, and the first and second variable domains of the antibody are VH and VL, respectively. In certain embodiments, the antibody binds the receptor on an outer surface of an immune cell, and the first and second variable domains of the antibody are VL and VH, respectively. In certain embodiments, the VH and the VL comprise Cys residues at positions 44 and 100, respectively, under Kabat numbering. In certain embodiments, the VH and the VL comprise Cys residues at positions 44 and 100, respectively, under Kabat numbering. In certain embodiments, the first and second variable domains of the TCR are Vα and Vβ, respectively. In certain embodiments, the first and second variable domains of the TCR are Vβ and Vα, respectively. In certain embodiments, the two polypeptides form a symmetric dimeric complex by pairing of the IgG Fc regions. In certain embodiments, the domains are connected by one or more linkers.

In certain embodiments, the soluble TCR fusion protein comprises (a) a first polypeptide chain comprising, from N-terminus to C-terminus, a single chain TCR variable fragment comprising the Vα and the Vβ of the TCR, an optional linker, a VH of an antibody, a CH1 domain, and a first IgG Fc region comprising hinge, CH2, and CH3 domains; (b) a second polypeptide chain comprising, from N-terminus to C-terminus, a VL of the antibody that pairs with the VH, and a light chain constant (CL) domain; and (c) a third polypeptide chain comprising a second IgG Fc region comprising hinge, CH2, and CH3 domains, wherein the first and second IgG Fc regions pair to form a dimer (see International Patent Application Publication No. WO 2021/163366A1). In certain embodiments, the antibody binds the receptor on an outer surface of an immune cell. In certain embodiments, the two polypeptides comprising the IgG Fc region form a complex by pairing of the IgG Fc regions, and the two polypeptides comprising the polypeptides comprising the VL and the VH domain of the antibody for a complex by pairing of the VL and the VH domains. In certain embodiments, within the single chain TCR variable fragment, the Vα is linked to the C-terminus of the Vβ via a linker.

IgG Fc region (e.g., human IgG Fc region), to the extent binding to FcRn, is also useful as a half-life extending domain. Where an Fc region is used for this purpose, it is contemplated that its CDC and/or ADCC effector function may not need to be retained. In certain embodiments, the soluble TCR fusion protein disclosed herein comprises an antibody Fc domain having low or no ADCC effector function. Without wishing to be bound by theory, it is contemplated that the presence of an Fc domain (e.g., an Fc domain that binds FcRn) may increase the serum half-life of the soluble TCR fusion protein and the low level or absence of ADCC effector function reduces the risk of killing the target CD8+ T cells. While the longer serum half-life could in theory increase both efficacy and toxicity, it is contemplated that the potential toxicity is mitigated by specific targeting of the TCR fusion protein to CD8-expressing cells. ADCC is mediated by binding to Fcγ receptors. Accordingly, in certain embodiments, the Fc domain incorporates one or more mutations or modifications, in either or both Fc polypeptide chains, that alter the binding to an Fcγ receptor (e.g., FcγRI/CD64, FcγRIIA/CD32A, FcγRIIB/CD32B, FcγRIIIIA/CD16, or FcγRIIIB).

Within the Fc domain, CD16 binding is mediated by the hinge region and the CH2 domain. For example, within human IgG1, the interaction with CD16 is primarily focused on amino acid residues Asp 265—Glu 269, Asn 297—Thr 299, Ala 327—Ile 332, Leu 234—Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction. All the amino acid positions in antibody Fc domains in this application are numbered according to the EU numbering system (see Edelman et al., (1969) Proc. Natl. Acad. Sci. USA 63:78-85; Kabat et al., Sequences of Proteins of Immunological Interest (5th ed. 1991)).

Mutations in IgG Fc domain and their impact on ADCC and CDC effector functions have been characterized (see, e.g., Wang et al., (2018) Protein Cell 9(1):63-73). In certain embodiments, the soluble TCR fusion protein comprising an antibody (e.g., IgG, e.g., human IgG1) Fc domain comprising one or more mutations that reduce ADCC and/or CDC effector functions. Exemplary Fc domain mutations that reduce ADCC and CDC effector functions are disclosed in U.S. Pat. No. 11,084,863. In certain embodiments, the antibody Fc region comprises a human IgG1 Fc region comprising one or more effector function silencing mutations, optionally at one or more of positions selected from 233, 234, 235, 236, 297, 327, 330, and 331, according to EU numbering. In certain embodiments, the antibody Fc domain differs from the wild-type human IgG1 Fc domain at one or more positions selected from R292, S298, E233P, L234, L235, G237, N297, S298, A327, P329, A330, P331, and K414. In certain embodiments, the antibody Fc domain differs from the human IgG1 Fc domain at one or more positions selected from E233P, L234, L235, G237, A327, P329, A330, and P331. In certain embodiments, the antibody Fc domain differs from the human IgG1 Fc domain at one or more positions selected from R292, S298, and K414. In some embodiments, the antibody Fc domain differs from the human IgG1 Fc domain at: (a) positions L234 and L235; (b) positions L234, L235, and P329; or (c) positions L234, L235, A327, A330, and P331. or (d) positions E233P, L234, L235, A327, A330, and P331.

In certain embodiments, the one or more mutations are selected from E233P, L234A, L235A, L235E, N297A, N297G, N297Q, S298A, A327G, P329A, P329G, A330S, and P331S. In certain embodiments, the soluble TCR fusion protein comprises a human IgG1 Fc comprising L234A and L235A substitutions. In certain embodiments, the soluble TCR fusion protein comprises a human IgG1 Fc comprising E233P, L234A, L235A, and P329G or P329A substitutions. In certain embodiments, the soluble TCR fusion protein comprises a human IgG1 Fc comprising N297A substitution. In certain embodiments, the soluble TCR fusion protein comprises a human IgG1 Fc comprising L234A, L235A, and N297A substitutions. In certain embodiments, the soluble TCR fusion protein comprises a human IgG1 Fc comprising E233P, L234A, L235A, N297A, and P329G or P329A substitutions. In certain embodiments, the soluble TCR fusion protein comprises a human IgG1 Fc comprising E233P, L234A, L235A, A327G, A330S, and P331S substitutions. In certain embodiments, the soluble TCR fusion protein comprises a human IgG1 Fc comprising L234A, L235A, N297A, A327G, A330S, and P331S substitutions.

Fc domains of human IgG4 and IgG2 are known to have little or no ADCC effector function. In certain embodiments, the soluble TCR fusion protein comprises a human IgG4 Fc domain or a fragment thereof. In certain embodiments, the IgG4 Fc domain comprises a S228P substitution. In certain embodiments, the soluble TCR fusion protein comprises a human IgG4 or IgG2 Fc region.

Where the format of a Fc-containing soluble TCR fusion protein disclosed herein is asymmetric, for example, in the Fab-Fc-immunomodulator (LC) format or the IgG-immunomodulator (lx HC) format, one or more mutations can be introduced into the Fc domain to promote heterodimerization. In certain embodiments, the first and second IgG Fc regions in the soluble TCR fusion protein comprise amino acid substitutions to promote Fc heterodimerization, for example at one or more positions selected from 347, 349, 351, 354, 356, 357, 360, 362, 364, 366, 368, 370, 390, 392, 394, 399, 400, 401, 405, 407, 409, 411 and 439, according to EU numbering. For example, one or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T3661, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y4071, Y407V, K409F, K409W, K409D, T411D, T411E, K439D, and K439E.

In certain embodiments, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V. This set of substitutions are referred to as “knobs-in-holes,” wherein the polypeptide chain comprising a T366W substitution has a “knob” and the polypeptide chain comprising T366S, L368A, and Y407V substitutions has a “hole.” Alternatively, amino acid substitutions could be selected from the following sets of substitutions shown in Table 4 below. Additional exemplary Fc domain substitutions that promote heterodimerization are disclosed in U.S. Pat. No. 11,084,863.

TABLE 4
Fc Heterodimerization Mutations

| | First Fc Polypeptide | Second Fc Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |
| Set 16 | K409W | D399V/F405T |
| Set 17 | Y349S | E357W |
| Set 18 | K360E | Q347R |
| Set 19 | K360E/K409W | Q347R/D399V/F405T |
| Set 20 | Q347E/K360E/K409W | Q347R/D399V/F405T |
| Set 21 | Y349S/K409W | E357W/D399V/F405T |
| Set 22 | T366K/L351K | L351D/L368E |
| Set 23 | T366K/L351K | L351D/Y349E |
| Set 24 | T366K/L351K | L351D/Y349D |
| Set 25 | T366K/L351K | L351D/Y349E/L368E |
| Set 26 | T366K/L351K | L351D/Y349D/L368E |
| Set 27 | E356K/D399K | K392D/K409D |
| Set 28 | L351Y, D399R, D399K, | T366V, T366I, T366L, T366M, |
| | S400K, S400R, Y407A, | N390D, N390E, K392L, K392M, |
| | Y407I, Y407V | K392V, K392F K392D, K392E, |
| | | K409F, K409W, T411D and |
| | | T411E |
| Set 29 | T350V, L351Y, F405A, | T350V, T366L, K392L, and |
| | and Y407V | T394W |

In certain embodiments, the structural stability of a hetero-multimeric protein may be increased by introducing S354C on either of the first or second polypeptide chain, and Y349C on the opposing polypeptide chain, which forms an artificial disulfide bridge within the interface of the two polypeptides.

Unless indicated otherwise, the amino acid substitutions described above are identified in the context of human IgG1. It is understood that corresponding substitutions are also contemplated in the context of human IgG2, IgG3, and IgG4. For example, the amino acid residue at position 234 of human IgG4 is F, whereas the amino acid residue at position 234 of human IgG1 is L. Where an L234A substitution is described in the context of human IgG1, the F234A substitution in the context of human IgG4 is also contemplated.

For the purpose of improving the pharmacokinetics of a TCR fusion protein, a different half-life extending domain can also be used. Examples of half-life extending domains include, but are not limited to serum albumin (e.g., human serum albumin), a binding domain of serum albumin (e.g., human serum albumin), and PEGylation. In certain embodiments, the human serum albumin binding domain is a polypeptide; for example U.S. Patent Application Publication No. US20130316952A1 discloses a polypeptide that binds serum albumin having the amino acid sequence of LKEAKEKAIEELKKAGITSDYYFDLINKAKTVEGVNALKDEILKA (SEQ ID NO: 237). Additional exemplary polypeptides that bind HSA are described in Dennis et al. (2002) J. Biol. Chem., 277: 35035-43; Jacobs et al. (2015) Protein Eng. Des. Sel., 28: 385-93; and Zorzi et al. (2017) Nat. Commun., 8: 16092.

In certain embodiments, the soluble TCR is fused with a cytokine, e.g., an immunostimulatory cytokine such as IL-2, IFN-γ, CCL21, GM-CSF, IL-12, IL-15, IL-6, IL-7, IL-18, IL-21, IL-23, or IL-27. Such fusion constructs can be used to target delivery of the cytokine to microenvironment of the cells targeted by the soluble TCR, thereby to modulate the immune status of the microenvironment.

In certain embodiments, the soluble TCR is conjugated or fused with a chemotherapeutic agent or a cytotoxic agent, thereby allowing targeted killing of the cells targeted by the soluble TCR. In certain embodiments, the soluble TCR is conjugated to a radioactive compound, prodrug activating enzyme (DT-diaphorase (DTD) or Biphenyl hydrolase-like protein (BPHL) for example), chemotherapeutic agent (cis-platin for example), toxin (Pseudomonas exotoxin such as PE38, calcimycin or diphtheria toxin for example), immune-modulating antibody fragment such as anti-CD3 or anti-CD16 for example, immune-modulating cytokine (IL-2 for example). In certain embodiments, the soluble TCR is conjugated to a compound selected from mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane.

In certain embodiments, the soluble TCR is conjugated or fused with a detectable label. For example, the soluble TCR may be conjugated (covalently or non-covalently) with or fused with a radioisotope, a fluorophore (e.g., fluorescein isothiocyanate (FITC), phycoerythrin (PE)), an enzyme (e.g., alkaline phosphatase, horseradish peroxidase), or a particle (e.g., a gold particle). Such molecules can be used in diagnostic screens to detect the presence of the cells targeted by the soluble TCR (e.g., cancer cells) within a subject.

In certain embodiments, the soluble TCR can also be provided in the form of a multimeric complex, comprising at least two soluble TCR molecules. In certain embodiments, multimerization of soluble TCRs is achieved by adding a multimerization domain to the TCR construct, for example a biotin, the tetramerisation domain of p53, or a tetramerisation domain of Haemoglobin. In certain embodiments, multimerization of soluble TCRs is achieved through adding streptavidin to two or more soluble TCR molecules that are each connected to a biotin moiety. Similar approaches known in the art for the generation of multimeric soluble TCR are also possible and included in this disclosure. Also provided are multimeric complexes comprising more than two soluble TCR of the invention.

The engineered TCRs disclosed herein include multiple components, which can be linked to each other by a peptide bond or a linker (e.g., peptide linker). In certain embodiments, the peptide linker does not comprise any multimerization or polymerization activity. A peptide linker disclosed herein can be used for example to link a VH and a VL in an scFv. Exemplary peptide linkers are described in U.S. Pat. Nos. 4,751,180 and 4,935,233 and International Application Publication No. WO198809344A1. In certain embodiments, the soluble TCR is linked to the binding domain for the immune cell receptor via a linker.

The linker can be a peptide linker, e.g., 5-50, 10-50, 15-50, 20-50, 25-50, 30-50, 35-50, 40-50, 45-50, 5-40, 10-40, 15-40, 20-40, 25-40, 30-40, 35-40, 5-30, 10-30, 15-30, 20-30, 25-30, 5-20, 10-20, or 15-20 amino acids in length. The linker sequence can be less than about 12, such as less than 10, or from 2-10 amino acids in length. Linker sequences are usually flexible, for example, when they are made up primarily of amino acids such as glycine, alanine and serine, which do not have bulky side chains likely to restrict flexibility. Alternatively, linkers with greater rigidity may be desirable. Examples of the linkers suitable for linking the domains in the fusion proteins include but are not limited to (GS)ₙ, (GGS)ₙ, (GGGS)ₙ, (GGSG)ₙ, (GGSGG)ₙ, and (GGGGS)ₙ, wherein n is 1, 2, 3, 4, 5, 6, 7, 8, 9, 10, 11, 12, 13, 14, 15, 16, 17, 18, 19, or 20. In certain embodiments, the peptide linker is a flexible linker having an amino acid sequence of (G₄S)₃ or (G₄S)₄. Other examples of suitable linkers that may be used to link a soluble TCR to a binding domain disclosed herein include, but are not limited to: GGGGS (SEQ ID NO: 238), GGGSG (SEQ ID NO: 239), GGSGG (SEQ ID NO: 240), GSGGG (SEQ ID NO: 241), GSGGGP (SEQ ID NO: 242), GGEPS (SEQ ID NO: 243), GGEGGGP (SEQ ID NO: 244), and GGEGGGSEGGGS (SEQ ID NO: 245) (as described in WO2010/133828), GGGGSGGGGSGGGGSGGGGSGGGS (SEQ ID NO: 246).

### IV. Engineered Immune Cells [Dag Reviewed 51-71]

In another aspect, the present disclosure provides an immune cell (e.g., T cell or NK cell) engineered to express a TCR disclosed herein.

### Types of Immune Cells

In some embodiments, the T cell is derived from blood, bone marrow, lymphoid organs (e.g., lymph nodes), or tumor biopsies from a subject. These cells typically are primary cells, such as those isolated directly from a subject and/or isolated from a subject. Other exemplary sources of T cells include T cell derived from stem cells, such as multipotent and pluripotent stem cells, including induced pluripotent stem cells (iPSCs). Stem cells can be directly isolated from a subject such as from cord blood, blood, or tissue. iPSCs can be derived from any cell type that can be used to produce iPSCs.

The T cell disclosed herein can be from a T cell subpopulation defined by function, activation state, maturity, potential for differentiation, expansion, recirculation, localization, and/or persistence capacities, antigen specificity, type of antigen receptor, presence in a particular organ or compartment, marker or cytokine secretion profile, and/or degree of differentiation. Among the subpopulations of T cells are naïve T (Tn) cells, effector T cells (Teff), memory T cells and sub-types thereof, such as stem cell memory T (Tscm), central memory T (Tcm), effector memory T (Tem), or terminally differentiated effector memory T cells, tumor-infiltrating lymphocytes (TIL), immature T cells, mature T cells, helper T cells, cytotoxic T cells, mucosa-associated invariant T (MALT) cells, naturally occurring and adaptive regulatory T (Treg) cells, helper T cells (e.g., TH1 cells, TH2 cells, TH3 cells, TH17 cells, TH9 cells, TH22 cells, follicular helper T cells), natural killer T (NKT) cells, alpha/beta T cells, and gamma/delta T cells. T cells are characterized by the expression of certain cell surface molecules such as the T cell receptor (TCR), a multiprotein complex that is involved in MHC binding.

A subtype of T cells are naïve T cells (Tn) that are characterized in that they have differentiated in bone marrow in vivo, and successfully undergone the central selection in the thymus. A single naïve T cell is able to generate multiple subsets of memory T cells with different phenotypic and functional properties and different gene expression profiles. For example, these cells can further differentiate into stem cell memory T cells, central memory T cells, or effector memory T cells (all described further herein). Included within this subtype are the naïve forms of helper T cells (CD4+) and cytotoxic T cells (CD8+). Naive T cells are commonly characterized by the surface expression of CD62L (L-selectin) and CCR7 (C—C chemokine receptor type 7); the absence of the activation markers CD25, CD44, or CD69; and the absence of the memory CD45RO isoform (see, e.g., De Rosa et al., Nat. Med. (2001) 7:245-248; van den Broek et al., Nat. Rev. Immunol. (2018) 18:363-373). They also express functional IL-7 receptors, consisting of subunits IL-7 receptor-α, CD127, and common-γ chain CD132.

Stem cell memory T cells (Tscm) are a subtype of T cells that typically comprises 2%-3% of the circulating T cell pool in vivo and can be identified within a naïve-like phenotype (CD45RA⁺CD45RO-CCR7⁺CD62L⁺CD27⁺CD28⁺) by expression of the memory marker CD95 (see, e.g., Gattinoni et al., Nat. Med. (2011) 17:1290-1297). Tscm cells can mount anamnestic responses and display gene transcript profiles encompassing features of both naïve T cells and central memory T cells. Moreover, Tscm cells are multipotent progenitors that can both self-renew and differentiate in vitro and in vivo into the entire spectrum of memory T cells, including central memory T cells and effector memory T cells (see, e.g., Ahmed et al., Cell Reports (2016) 17:2811-2812).

Central memory T cells (Tem) are a subtype of T cells characterized by the expression of CD45RO, CCR7, and CD62L. Tcm cells also have intermediate to high expression of CD44, which can be used to distinguish Tn cells from Tcm cells. Included within this subtype are Tcm forms of helper T cells (CD4⁺) and cytotoxic T cells (CD8⁺). Tcm cells are commonly found in the lymph nodes and in the peripheral circulation. Tcm cells have the ability to self-renew due to high levels of phosphorylation of the transcription factor STAT5. Tscm and Tcm cells are longer-lived and more proliferative than effector memory T cells or effector T cells.

Effector memory T cells (Tem) express CD45RO but lack expression of CCR7 and CD62L. They also have intermediate to high expression of CD44. CD62L acts as a “homing receptor” for lymphocytes to enter secondary lymphoid tissues. Thus, Tem cells are typically found in the peripheral circulation and tissues, rather than in the lymph nodes, and exhibit immediate effector function. In response to antigen stimulation, Tem cells proliferate and differentiate into CD62L⁻ effector T cells. Effector T cells (Teff) are fully differentiated T cells. Effector T cells are short-lived cells, as opposed to memory cells which have a potential of long-term survival but have strong cytotoxic activity.

Regulatory T cells (Tregs) are a specialized subpopulation of T cells that act to suppress immune response, thereby maintaining homeostasis and self-tolerance. Tregs are able to inhibit T cell proliferation and cytokine production and play a critical role in preventing autoimmunity.

Cytotoxic T lymphocytes (CTLs) are T cells that have the ability to kill a target cell. CTL activation can occur when two steps occur: 1) an interaction between an antigen-bound MHC molecule on the target cell and a T cell receptor on the CTL is made; and 2) a costimulatory signal is made by engagement of costimulatory molecules on the T cell and the target cell. CTLs then recognize specific antigens on target cells and induce the destruction of these target cells, e.g., by cell lysis.

Tumor infiltrating lymphocytes (TILs) are lymphocytes that have migrated into a tumor. In some embodiments, TILs can be cells at different stages of maturation or differentiation, e.g., TILs can include CTLs, Tregs, and/or effector memory T cells, among other types of lymphocytes.

In certain embodiments, the present disclosure provides a population of T cells that recognize one or more T cell epitopes disclosed herein. In some embodiments, the T cells include one or more subsets of T cells, such as a whole T cell population, CD4+ cells, CD8+ cells, or subpopulations and combinations thereof.

In certain embodiments, the T cell composition comprises CD8+ T cells. In certain embodiments, at least 30%, at least 40%, at least 50%, at least 60%, at least 70%, at least 80%, or at least 90% of the cells in the immune cell composition are CD8+ T cells. In certain embodiments, the immune cell composition further comprises CD4+ T cells, optionally wherein at least 20%, at least 30%, at least 40%, or at least 50% of the cells in the immune cell composition are CD4+ T cells. In some embodiments, the disclosure provides for a mixture of T cells, such as a mix of CD8+ and CD4+ cells. In certain embodiments in a mixture of CD8+ and CD4+ cells, the ratio of CD8+ to CD4+ cells can be, for example, 1:1, 1:2 to 2:1, 1:3 to 3:1, 1:4 to 4:1, 1:5 to 5:1, etc. Methods to measure the ratio of T cells are known in the art. For example a ratio of CD8+ to CD4+ can be determined by staining the CD8+ and CD4+ cells with CD8 and CD4 specific antibodies and characterize and enumerate the cells for example with Fluorescence-activated Cell Sorting (FACS). In certain embodiments, the T cell composition comprises alpha/beta T cells, and gamma/delta T cells. In some embodiments, the at least 30%, at least 40%, at least 50%, at least 60%, at least 70%, at least 80%, at least 90%, at least 95% of the cells in the immune cell composition are alpha/beta T cells. In some embodiments, the at least 1%, at least 2%, at least 3%, at least 4%, at least 5%, at least 6%, at least 7%, at least 8%, at least 9%, or at least 10% of the cells in the immune cell composition are gamma/delta T cells. For example a ratio of alpha/beta T cells, and gamma/delta T cells can be determined by staining the alpha/beta T cells and gamma/delta T cells with alpha/beta and gamma/delta specific antibodies and characterize and enumerate the cells for example with Fluorescence-activated Cell Sorting (FACS).

In some embodiments, the T cells are harvested from blood, bone marrow, lymphoid organs (e.g., lymph nodes), or tumor biopsies from a subject. In some embodiments the T cells are harvested from a cell culture. The T cells can be purified before further culturing and expansion or can be used in a mixture with other cells. In certain embodiments, the primary T cells and stem cell derived T cells can be used fresh or frozen. The T cells can be maintained in ex vivo culture in the presence of one or more cytokines, such as IL-15 and/or IL-12, and optionally one or more of IL-21, IL-7, IL-2 and IL-6. In some embodiments, the APCs loaded with T cell epitopes disclosed herein and prepared in accordance with the methods disclosed herein can be used to prime and expand certain T cells populations in vitro. Methods for T cell priming and expansion are known in the art, e.g., co-culturing the lymphocyte-rich fraction of the PBMCs with the APCs disclosed herein to expand T cells that are reactive to the T cell epitopes disclosed herein.

With reference to the subject to be treated, the T cells may be allogeneic and/or autologous. Allogeneic T cells are suitable for use in off-the-shelf methods. In certain embodiments, an off-the-shelf method employs pluripotent and/or multipotent cells, such as stem cells and induced pluripotent stem cells (iPSCs), for producing T cells. In some embodiments, the present disclosure provides an autologous method that includes isolating cells from a subject, preparing, processing, culturing, and/or engineering the cells as described herein to produce T cells, and administering the T cells to the patient, with or without cryopreservation prior to the administration. In some embodiments, the T cells are autologous T cells. In some embodiments, the T cells are allogeneic T cells (e.g., cells from a healthy donor).

Also provided herein are populations of T cells reactive to the T cell epitopes disclosed herein and compositions containing such T cells. In some embodiments, a composition or population disclosed herein is enriched for such T cells. For example, in certain embodiments, at least 1%, 5%0, 10%, 20%, 30%, 40%, 50%, 60%, 70%, 80%, 90%, 91%, 92%, 93%, 94%, 95%, 96%, 97%, 98%, or 99% of the total cells in the composition or population recognize one or more T cell epitopes disclosed herein. In certain embodiments, at least 1%, 5%, 10%, 20%, 30%, 40%, 50%, 60%, 70%, 80%, 90%, 91%, 92%, 93%, 94%, 95%, 96%, 97%, 98%, or 99% of the CD8+ cells in the composition or population recognize one or more T cell epitopes disclosed herein. In certain embodiments, at least 1%, 5%, 10%, 20%, 30%, 40%, 50%, 60%, 70%, 80%, 90%, 91%, 92%, 93%, 94%, 95%, 96%, 97%, 98%, or 99% of the CD4+ cells in the composition or population recognize one or more T cell epitopes disclosed herein.

The immune cell can also be an NK cell, e.g., a cytotoxic NK cell or an adaptive NK cell. NK cells appear typically CD56+(dim or bright) and CD16+ but not CD3+ in flow cytometry. Adaptive NK cells, also known as memory-like NK cells, are typically express CD57+ and lack many transcription factors and signaling proteins, including FCRγ, PLZF, Siglec-7, EAT-2, and SYK. In some embodiments, isolated subpopulations of CD56+NK cells comprise expression of NKG2C and CD57. In some embodiments, isolated subpopulations of CD56+NK cells comprise expression of at least one of CD57, CD16, NKG2A, NKG2B NKG2C, NKG2D, natural cytotoxicity receptors NCR (for example NKp30, NKp40, NKp44, NKp46), activating and inhibitory KIRs, and DNAM-1.

In some embodiments, the NK cell is derived from blood, cord blood, bone marrow, lymphoid organs (e.g., lymph nodes), or tumor biopsies from a subject. These cells typically are primary cells, such as those isolated directly from a subject and/or isolated from a subject. Various ways of dissociating cells from tissues or cell mixtures to separate the various cell types have been developed in the art. In some cases, these manipulations result in a relatively homogeneous population of cells. Other exemplary sources of NK cells include NK cells derived from stem cells, such as multipotent and pluripotent stem cells, including induced pluripotent stem cells (iPSCs). Stem cells can be directly isolated from a subject such as from cord blood, blood, or tissue. iPSCs can be derived from any cell type that can be used to produce iPSCs. The NK cells can be isolated by a sorting or selection process as described herein or by other methods known in the art. The proportion of NK cells in the isolated population may be higher than the proportion of NK cells in the natural source by at least about 10%, about 20%, about 30%, about 40%, about 50%, about 60%, about 70%, about 80%, about 85%, about 90%, or about 95%.

A “natural killer T cell” or “NKT cell” refers to a CDld-restricted T cell, which expresses a T cell receptor (TCR). Unlike a conventional T cell that detects peptide antigens presented by conventional major histocompatibility (MHC) molecules, an NKT cell recognizes lipid antigens presented by CDld, a non-classical MHC molecule. Two types of NKT cells are currently recognized. Invariant or type I NKT cells express a very limited TCR repertoire a Va24-Ja18 chain paired with Vb11 chain in humans, and is specific for glycolipid antigens. The second population of NKT cells, called nonclassical or noninvariant type II NKT cells, display a more heterogeneous TCR αβ usage. Adaptive or invariant (type I) NKT cells can be identified with the expression of at least one or more of the following markers, TCR Va24-Jal 8, Vb11, CD3, CD4, CD8, CD161, CD94, and CD56.

It is understood that in order to engineer the genome of a given type of immune cell, a cell higher up in the differentiation lineage (e.g., a progenitor cell, a hematopoietic stem cell, a CD34+ cell, or an induced pluripotent stem cell) can be engineered and the cell can be differentiated into the desirable cell type (see Dahlberg et al., Blood (2011) 117(23): 6083-6090 and Tajer et al., Cells (2019) 8(2): 169; also reviewed in A. De Los Angeles et al., Nature. (2015) 525(7570):469-78).

### Immune Cell Engineering

In TCR immune cell technologies, a TCR is used to confer immune cells the ability to target a specific epitope, e.g., an HPV-16 E2 T cell epitope. A cell can be engineered to express a recombinant TCR, for example, by introducing a nucleic acid sequence or a vector that encodes the TCR. It is understood that the cell can be a T cell or another type of immune cell, such as an NK cell. Accordingly, the present disclosure also provides an immune cell (e.g., T cell) expressing (e.g., recombinantly expressing) a TCR (e.g., engineered TCR) disclosed herein. Where an embodiment in this subsection is described in the context of a T cell, a similar embodiment is also contemplated where the T cell is replaced by another immune cell (e.g., a NK cell).

Methods for expressing a TCR in T cells are known in the art (see, e.g., U.S. Pat. No. 11,033,584). Methods for preparing TCR-transgenic T cells are known in the art and disclosed, for example, by Rath and Arber (2020) Cells 9:1485 and Xu et al., (2020) J. Cellular Immunol. 2(6):284-88). In certain embodiments, a immune cell therapy (for example a T cell therapy) comprises T cells having a plurality of (e.g., 2, 3, 4, 5, 6, 7, 8, 9, 10, or more) of different TCRs disclosed herein.

The present disclosure also provides one or more nucleic acids encoding a TCR (e.g., engineered TCR) disclosed herein. In certain embodiments, where the TCR comprises two or more separate polypeptide chains, they can be encoded by two or more separate nucleic acid sequences. In certain embodiments, a TCR comprising two or more separate polypeptide chains is encoded by a single nucleic acid sequence, where the coding sequences are linked by a ribosomal skipping element (see e.g., Walseng et al. PLOS ONE (2015) 10(4):e0119559). Ribosomal skipping sequences (also called self-cleaving peptides) are short (18-22 aa) viral sequences that can be introduced in-frame with a promoter of an endogenously or exogenously expressed gene. Commonly used ribosomal skipping sequences include T2A, P2A, E2A, and F2A. For a description of ribosomal skipping sequences and uses thereof, see, e.g., Chng et al. (MAbs, 2015, 7(2):403-412). Also provided is an immune cell (e.g., T cell, NK cell) comprising one or more of the foregoing nucleic acids encoding the TCR.

The present disclosure also provides an expression vector comprising the one or more nucleic acids. In certain embodiments, the expression vector comprises an expression regulatory element operably linked to the TCR coding sequence(s). Exemplary expression regulatory elements include transcriptional and/or translational control sequences, such as promoter, enhancer, transcription termination signal (e.g., polyadenylation signal), and internal ribosomal entry sites (IRES). Expression regulatory elements include those that direct constitutive expression of a nucleotide sequence in many types of host cells and those that direct expression of a nucleotide sequence only in certain host cells (e.g., tissue-specific regulatory sequences). A tissue-specific promoter may direct expression primarily in a desired tissue of interest, such as T lymphocytes. Expression regulatory elements may also direct expression in a temporal-dependent manner. In certain embodiments, the vector comprises a retroviral Rous sarcoma virus (RSV) LTR promoter (optionally with a RSV enhancer), a cytomegalovirus (CMV) promoter (optionally with a CMV enhancer), a SV40 promoter, a murine stem cell virus (MSCV) promoter, a dihydrofolate reductase promoter, a β-actin promoter, a phosphoglycerol kinase (PGK) promoter, a EF1α promoter, or a synthetic MND promoter operably linked to the TCR coding sequence. In certain embodiments, the vector further comprises an enhancer element (e.g., a CMV enhancer, a SV40 enhancer), a woodchuck hepatitis virus post-transcriptional regulatory element (WPRE), a R-U5′ segment in LTR of HTLV-I (see, e.g., Takebe et al. (1988) MOL. CELL. BIOL., 8: 466), and/or an intron sequence between exons 2 and 3 of rabbit β-globin (see, e.g., O'Hare et al. (1981) PROC. NATL. ACAD. SCI. USA., 78: 1527). Exemplary polyadenylation signals comprise SV40, hGH, BGH, and rbGlob. It will be appreciated by those skilled in the art that the design of the expression vector can depend on factors such as the choice of the host cell to be transformed, the level of expression desired, etc. In certain embodiments, the expression vector is a viral vector (e.g., a lentiviral vector, an AAV vector).

In an additional aspect, the disclosure provides a viral vector comprising a nucleic acid encoding a TCR disclosed herein. Also provided is an immune cell (e.g., T cell, NK cell) comprising the vector. In certain embodiments, the viral vector comprises a retrovirus such as a gamma retrovirus or lentivirus, or an Adeno-Associated Virus (AAV). AAVs have a single-stranded deoxyribonucleic acid (ssDNA) and have a 5′ and 3′ inverted terminal repeat sequence. In some embodiments, the Adeno-Associated Virus comprises a capsid of AAV1 serotype, AAV2 serotype, AAV3 serotype, AAV4 serotype, AAV5 serotype, AAV6 serotype, AAV7 serotype, AAV8 serotype, or AAV9 serotype. In some embodiments, the AAV comprises a capsid of an engineered pseudoserotype. In some embodiments, the AAV capsid has tropism for the target cell (e.g., T cell, NK cell), for example, an AAV6 capsid.

In certain embodiments, the TCR coding sequence in an expression vector (e.g., viral vector) is flanked by homology arms, thereby facilitating site-specific integration of the TCR coding sequence into the genome of the target cell. To increase genomic integration efficiency, a site-directed DNase (e.g., a CRISPR-Cas system) can be delivered to the target cell, cutting the integration site or a nearby sequence and eliciting homology-directed repair (see, e.g., Eyquem et al. Nature (2017) 543:113-117). An exemplary AAV expression construct comprises, from 5′ to 3′, a 5′ ITRs, a left homology arm, a DNA sequence to be inserted, a right homology arm, and a 3′ ITR. The DNA sequence can comprise a TCR disclosed herein and optionally a promoter, an enhancer, a ribosomal skipping sequence, a transcription termination signal, a polyadenylation signal, or combinations thereof. In certain embodiments, the TCR coding sequence can be inserted at a locus in the target cell genome such that it is operably linked to an endogenous promoter, thereby expressing the TCR by the endogenous promoter.

Site-directed DNases include but are not limited to CRISPR-Cas nucleases, TALENs, ZnF nucleases, and Meganucleases. CRISPR-Cas nucleases are known in the art and comprise for example RNA guided Class 2 Type II and V nucleases, and Class 1 Type I nucleases. CRISPR-Cas nucleases typically comprise a CRISPR-Cas protein component and an RNA guide component.

A typical Class 2, Type II CRISPR-Cas nuclease system is a CRISPR-Cas9 system. A CRISPR-Cas9 system can include a Cas9 protein, a crRNA and a tracrRNA. The crRNA has a region of complementarity to a potential target DNA sequence and a second region that forms base-pair hydrogen bonds with the tracrRNA. The region of complementarity to the target DNA sequence is called “spacer.” Complex formation between crRNA/tracrRNA and a Cas9 protein results in conformational change of the Cas9 protein that facilitates binding to DNA, endonuclease activities of the Cas9 protein, and crRNA-guided site-specific DNA cleavage by the Cas9 endonuclease. The cleavage also requires that the target DNA sequence is adjacent to a cognate protospacer adjacent motif (PAM) of the Cas9 protein. By engineering a crRNA to have an appropriate spacer sequence that targets a DNA sequence adjacent to a PAM, a CRISPR-Cas complex can be targeted to cleave at a locus of interest, e.g., a locus at which sequence modification is desired. The guide nucleic acid can be a dual guide (separate crRNA and tracrRNA sequences) or an engineered single guide (fused crRNA and tracrRNA sequences).

Class 2, Type V CRISPR-Cas nuclease systems can involve a Cpf1/Cas12a, Cas2b (formerly C2c1), or Cas12c (formerly C2c3) nuclease (see, e.g., Zetsche et al., Cell (2015) 163:759-71; Shmakov et al., Molecular Cell (2015), 60(3):385-97). A native Cpf1/Cas12a system only includes a crRNA and not a tracrRNA. The crRNA, as the single guide nucleic acid, can hybridize with a target DNA sequence and direct a Cpf1 nuclease to cleave the DNA.

In at least Class 2 systems, the guide nucleic acid can comprise RNA, DNA, and/or analogs thereof. Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA™) (Exiqon, Woburn, MA) nucleosides, glycol nucleic acid, bridged nucleic acids, and morpholino structures.

CRISPR-Cas nucleases in combination with AAV virus can be used for site directed introduction of donor sequences into a genome of a cell. For example, the CRISPR-Cas nuclease can be used to introduce a site directed genomic cleavage in a genomic locus. Then, an AAV vector comprising a nucleic acid encoding a TCR (e.g., engineered TCR), flanked by homology arms, can be used to insert the TCR-coding sequence into or near the cleavage site.

In some embodiments, the T cell disclosed herein has reduced or diminished expression of an endogenous gene, such as an endogenous TCR, a MHC class I or II, B2M, or an immune checkpoint gene such as PDCD1. The disruption of gene expression can be achieved by genomic DNA cleavage using a site-directed DNase, such as a CRISPR-Cas system, followed by non-homologous end joining (NHEJ) that leads to small insertions or deletions often causing frame-shifting. Alternatively, gene expression disruption can result from homology-directed repair, where a repair template carrying one or more mutations (e.g., nonsense mutations) is provided.

Adverse effects of T cell therapy can include cytokine release syndrome and prolonged B-cell depletion. Introduction of a suicide/safety switch in the recipient cells may improve the safety profile of a cell-based therapy. Accordingly, the cells may be genetically modified to include a suicide/safety switch. The suicide/safety switch may be a gene that confers sensitivity to an agent, e.g., a drug, upon the cell in which the gene is expressed, and which causes the cell to die when the cell is contacted with or exposed to the agent. Exemplary suicide/safety switches are described in e.g., in Protein Cell. 2017 August; 8(8): 573-589. In certain embodiments, the suicide/safety switch comprises HSV-TK, which comprises a thymidine kinase, which can kill the cells expressing the thymidine kinase upon administration of the prodrugs ganciclovir or acyclovir. The tk gene is a cell cycle-dependent suicide gene that catalyzes the generation of triphosphate ganciclovir (GCV), which is toxic to proliferating cells by inhibiting DNA chain elongation. In certain embodiments, the suicide/safety switch comprises cytosine deaminase 5-fluorocytosine. Cytosine deaminase converts the prodrug 5-fluorocytosine to the active 5-fluorouracil, which in turn causes cell death. In certain embodiments, the suicide/safety switch comprises inducible FAS (iFAS) and inducible Caspase 9 (iCasp9) that dimerize in the presence of the chemical rimiducid or rimiducid analogues (see, e.g., U.S. Patent Application Pub. No. US20170166877 A1). In certain embodiments, the suicide/safety switch comprises purine nucleoside phosphorylase that converts the prodrug, fludarabine to 2-fluoroadenine, or nitroreductase, which renders host cells susceptible to the drug CB1954. In certain embodiments, the suicide/safety switch comprises a transmembrane protein called RQR8 which includes epitopes of CD20, which can kill the cells expressing RQR8 upon administration of rituximab (see, e.g., Haematologica. (2009) 94(9): 1316-20).

In certain embodiments, a T cell disclosed herein further expresses a marker or comprises a nucleic acid capable of expressing a marker. The marker can be a cell surface marker, which may be used to confirm transduction or engineering of the cell to express the TCR. In certain embodiments, the cell surface marker comprises a fragment of a cell surface receptor, such as a truncated EGFR (tEGFR). In certain embodiments, the marker includes all or part (e.g., truncated form) of CD34, a NGFR, or epidermal growth factor receptor (e.g., tEGFR). In certain embodiments, the marker is covalently linked to the TCR when expressed. In other embodiments, the marker is encoded by the same vector as the TCR but in a different open reading frame (ORF), for example, separated from the TCR coding sequence by an internal ribosomal entry site (IRES). In other embodiments, the marker is encoded by the same vector as the TCR from the same ORF, for example, linked to the TCR coding sequence by a polynucleotide encoding a cleavable linker sequence or a ribosomal skipping sequence, e.g., T2A or P2A (see WO2014031687). For example, the marker can be a truncated EGFR (tEGFR) that is linked to the TCR coding sequence by a T2A ribosomal skipping sequence.

### V. Pharmaceutical Compositions and Therapeutic Methods

Disclosed herein are pharmaceutical compositions that contain one or more engineered TCRs (for example a soluble TCR fusion protein) or engineered immune cells expressing an engineered TCR described herein. Also disclosed are therapeutic uses of these pharmaceutical compositions.

### Engineered TCR Compositions

In some embodiments, the pharmaceutical composition comprises a pharmaceutically acceptable carrier, excipient, or stabilizer (see, e.g., Adeboye Adejare, Remington: The Science and Practice of Pharmacy (23d ed. 2020)). Suitable carriers are well known in the art and may include for example, nanoparticles, nanotubes, dendrimers, liposomes, foams, hydrogels, cubosomes, quantum dots, natural drug carriers, exosomes, and macrophages. Acceptable carriers, excipients, or stabilizers are nontoxic to the recipients at the dosages and concentrations, and may comprise phosphate buffered saline solutions, water, emulsions, such as oil/water emulsions, various types of wetting agents, and sterile solutions. In some embodiments, the carrier or excipient comprises buffers such as phosphate, citrate, and other organic acids; antioxidants including ascorbic acid and methionine; preservatives (such as Mercury((o-carboxyphenyl)thio)ethyl sodium salt (THIO E SAL), octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; β-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG), TWEEN or PLURONICS.

Alternatively or in addition, the pharmaceutical composition may comprise one or more immunogenicity enhancing adjuvants (also referred to as “adjuvants” herein). Such adjuvants are substances that enhance or potentiate the immune response (e.g., immune responses mediated by CD8-positive T cells and helper-T (TH) cells to an antigen) in a non-antigen-specific manner, and would thus be considered useful in a pharmaceutical composition disclosed herein. Suitable adjuvants include, but are not limited to, 1018 ISS, aluminum salts, AMPLIVAX®, AS15, BCG, CP-870,893, CPG7909, CyaA, dSLIM, flagellin or TLR5 ligands derived from flagellin, FLT3 ligand, GM-CSF, IC30, IC31, lmiquimod (ALDARA®), resiquimod, IMUFACT®, IMP321, interleukins as IL-2, IL-13, IL-21, interferon-α or -β, or pegylated derivatives thereof, IS Patch, ISS, ISCOMATRIX, ISCOMs, JUVIMMUNE®, LIPOVAC®, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK®, OspA, poly(lactide coglycolide) [PLG]-based and dextran microparticles, talactoferrin SRL 172, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, QUIL®, or Superfos. Depending upon the circumstances, adjuvants such as Freund's or GM-CSF may be preferred. Several immunological adjuvants (e.g., MF59) specific for dendritic cells and their preparation have been described previously (Ott et al., Pharm Biotechnol. (1995) 6:277-96). Also, certain cytokines may be used. Several cytokines have been directly linked to influencing dendritic cell migration to lymphoid tissues (e.g., TNFα), accelerating the maturation of dendritic cells into efficient antigen-presenting cells for T-lymphocytes (e.g., GM-CSF, IL-1 and IL-4) (see, U.S. Pat. No. 5,849,589) and acting as adjuvants (e.g., IL-12, IL-15, IL-23, IL-7, IFN-α, IFN-β) (see Gabrilovich et al., 1996). In certain embodiments, an adjuvant is a naturally occurring adjuvant, such as a cytokine in its wild-type form. In certain embodiments, an adjuvant is a non-naturally occurring adjuvant.

In certain circumstances, the antigen peptides of the pharmaceutical composition disclosed herein can be conjugated to a carrier protein or presented in a multimeric format such as virus-like particles or nanoparticles. Such strategies can boost immune responses by increasing the half-life of the epitope by decreasing renal clearance and susceptibility to proteolytic degradation. Linkage to carrier proteins can be achieved by chemical conjugation.

Pharmaceutical compositions comprising such adjuvants, carriers, and/or excipients can be formulated by well-known conventional methods. In some embodiments, the pharmaceutical composition is formulated to have a pH in the range of about 4.5 to about 9.0, including for example pH ranges of about any one of 5.0 to about 8.0, about 6.5 to about 7.5, or about 6.5 to about 7.0. In some embodiments, the pharmaceutical composition can also be made to be isotonic with blood by the addition of a suitable tonicity modifier, such as glycerol. In some embodiments, the pharmaceutical composition is formulated as a liquid or the liquid is dried for a solid preparation (for example by spray drying or lyophilization). The lyophilization process is aimed at removal of water and typically involves freezing, primary drying, and secondary drying. To safeguard the peptide molecules from such stresses, typically cryoprotectants are used to improve viability and structural stability. Cryoprotectants include, but are not limited to DMSO, sugars like trehalose and sucrase, polysaccharides, like starch and dextran, or proteins. Solid preparations allow for a long storage time of the pharmaceutical composition. Lyophilized preparations of the pharmaceutical composition can be stored in bulk or in ready-made doses for use such as in a vial. The content of peptide for a one dose vial may be 1, 2, 3, 4, 5, 6, 7, 8, 9, 10, 11, 12, 13, 14, 15, 16, 17, 18, 19, or 20 mg. The content of peptide for a one dose vial is preferably 1.0 to 20 mg, 2.0 to 15 mg, 5.0 to 14 mg, 10 to 13 mg, or 12 mg. Before administration to a patient, the solid preparation can be dissolved with a suitable solvent such as water.

These pharmaceutical compositions can be administered to the subject at a suitable dose. It is preferred that the pharmaceutically acceptable carrier be one which has no detrimental side effects or toxicity under the conditions of use. Preferably, a pharmaceutical composition is sterile and produced according to GMP guidelines.

In some embodiments, the pharmaceutical composition is in the form of a lyophilized formulation or an aqueous solution. The pharmaceutical composition can be in dosages suspended in any appropriate pharmaceutical vehicle or carrier in sufficient volume to carry the dosage. Generally, the final volume, including carriers, adjuvants, and the like, typically will be at least 0.5 mL. The upper limit is governed by the practicality of the amount to be administered, generally in the range of about 0.5 ml to about 2.0 ml. In some embodiments, the pharmaceutical composition is administered to a patient enterally or parenterally. Depending upon the circumstances, the pharmaceutical composition can be administered to a patient by oral, sublingual, gastric, or rectal administration. Alternatively, the pharmaceutical composition can administered to a patient by intravenous, intramuscular, intratumoral, intradermal, intrajejunal, intraileal, intracolonic, or intrarectal administration. The pharmaceutical composition can be also delivered by subcutaneous axillary and/or inguinal injection.

### Immune Cell Therapies

The engineered immune cell compositions disclosed herein are useful as immune cell therapies (e.g., adoptive T cell or NK cell therapy). Accordingly, in another aspect, the present disclosure provides an immune cell therapy comprising one or more engineered immune cell compositions disclosed herein.

In certain embodiments, the immune cell therapy is autologous, i.e., T cells obtained from a patient, after in vitro culture, are administered to the same patient. In certain embodiments, the immune cell therapy is allogeneic and the immune cells are obtained from a healthy donor, optionally wherein the T cells are genetically engineered to inactivate a component of class I MHC (e.g., 02M).

An immune cell therapy can be provided as a cell composition. It is understood that other types of cells, such as APCs, may be used for expanding T cells ex vivo. As such, the cell composition may include other cell types in addition to T cells. In certain embodiments, the cell composition has been enriched for T cells. Where the T cells are prepared by stimulation using APCs in an ex vivo cell culture, the T cells can be enriched by methods known in the art. For example, in certain embodiments, the APCs are removed from the cell culture by surface marker-based magnetic bead selection or cell sorting. In certain embodiments, the APCs are outgrown by T cells under conditions (e.g., cytokines) that preferably support T cell proliferation. In certain embodiments, the APCs are removed by their stronger adherence to tissue culture plate than T cells. The enrichment can produce a composition in which at least 70%, at least 80%, at least 90%, at least 95%, or at least 99% of the cells in the composition are T cells. In certain embodiments, the composition is substantially free of myeloid cells. For example, in certain embodiments, the percentage of myeloid cells relative to all cells in the composition is 20% or less, 10% or less, 5% or less, 4% or less, 3% or less, 2% or less, or 1% or less.

The immune cell therapy compositions can further comprise one or more carriers, excipients, and/or immunogenicity enhancing adjuvants. Exemplary adjuvants, carriers, and excipients are described herein (see the “Engineered TCR compositions” subsection above). In certain embodiments, the immunogenicity enhancing adjuvant comprises an immunostimulator, such as an immunostimulatory fusion protein and/or a protein nanogel. Exemplary immunostimulatory fusion proteins useful in combination with T cell therapies disclosed herein are described in WO2019010219. Exemplary protein nanogels useful in combination with T cell therapies are described in WO2015048498, WO2017218533, WO2019050978, and WO2019050977. In certain embodiments of the T cell therapy, T cells loaded with an immunostimulatory fusion protein and T cells loaded with a protein nanogel are used in combination, in separate compositions or in a single composition. In a specific embodiment, a T cell is loaded with both the immunostimulatory fusion protein and the nanogel. Methods of preparing such co-loaded T cell composition are described, for example, in WO2020205808.

### Treatment of Cancer

The TCRs disclosed herein bind HPV-16 epitopes, which are often presented on the surface of cancer cells infected with HPV-16. Accordingly, the pharmaceutical compositions disclosed herein are useful in treating a proliferative disorder such as cancer that is HPV-16 positive. Accordingly, the present disclosure provides methods of treating a proliferative disorder (e.g., cancer) comprising administering a therapeutically effective amount of a pharmaceutical composition disclosed herein to a subject in need thereof.

In certain embodiments, the present disclosure provides a method of treating cancer. In certain embodiments, the cancer to be treated is a solid cancer, such as brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In certain embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor. In certain embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

In certain embodiments, the cancer is head and neck squamous cell carcinoma (HNSCC). In certain embodiments, the cancer is HPV⁺ (e.g., HPV-16⁺) HNSCC. In certain embodiments, the cancer is HPV⁺ (e.g., HPV-16⁺) Oropharyngeal Squamous Cell Carcinoma OPSCC). HPV status of a cancer can be assessed by taking a biopsy of the tumor, extracting the DNA and testing for viral DNA via a PCR based assay. The HPV can be genotyped with a kit for example a AmoyDx® Human papillomavirus (HPV) Genotyping Detection Kit (Amoy Diagnostics Cβ., LTD, China). Tissue biopsies can be also tested with antibody staining for the presence of P16, a viral protein expressed in cells that are HPV positive. It has been discovered that HPV proteins such as E1 and E2 are expressed in HPV⁺ (e.g., HPV-16⁺) HNSCC or OPSCC and is recognized by certain cytotoxic T-cell clonotypes. Accordingly, in certain embodiment, the present disclosure provides a method of treating HPV⁺ (e.g., HPV-16⁺) HNSCC or OPSCC using any of the pharmaceutical compositions disclosed herein.

In certain embodiments, the cancer is selected from any of the cancer indications above and is HPV⁺ (e.g., HPV-16⁺). In certain embodiments, the HPV⁺ cancer is treated with a therapy disclosed herein that is targeted for one or more antigens of the HPV strain (for example E1 or E2).

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. In certain embodiments, delivery is such that the reduction of a symptom, or other parameter related to the disorder is greater than what would be observed with one treatment delivered in the absence of the other. The effect of the two treatments can be partially additive, wholly additive, or greater than additive.

The disclosure provides a method of treating a subject by the administration of a second therapeutic agent in combination with one or more of the therapies disclosed herein. Exemplary therapeutic agents that may be used as part of a combination therapy in treating cancer, include, for example, radiation, chemotherapy, surgery, immunotherapy (for example immune checkpoint inhibitor, or cell therapy), stem cell or bone marrow transplant, or hormone therapy. It is understood that the pharmaceutical compositions disclosed herein, which are designed to provide or activate immune cells (e.g., T lymphocytes), may, under certain circumstances, cause side effects such as neurotoxicity under certain conditions. Accordingly, in certain embodiments, the second therapeutic agent that can be used in combination comprises an agent that mitigates a side effect of the immunostimulatory fusion protein, e.g., reduces neurotoxicity.

The amount of the pharmaceutical composition and the amount of the additional therapeutic agent, and the relative timing of administration, may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, simultaneously, or together. Further, for example, the pharmaceutical composition may be administered during a time when the additional therapeutic agent(s) exerts its prophylactic or therapeutic effect, or vice versa.

### EXAMPLES

The invention now being generally described, will be more readily understood by reference to the following examples, which are included merely for purposes of illustration of certain aspects and embodiments of the present invention, and is not intended to limit the invention.

### Example 1. Methods

This example describes the methods used in the subsequent examples.

### Patient Selection and Clinical Annotation

Patients with newly diagnosed HPV-driven OPSCC (base of tongue, tonsil, and unknown primary) were identified and prospectively enrolled over a 12-month study period if they were undergoing confirmatory tissue biopsy or definitive oncologic transoral robotic-assisted resection with neck management of the primary tumor and/or involved cervical neck nodes. Patients with known distant metastatic disease were excluded, otherwise all clinical stages of curable disease were eligible (stages I, II, and III by American Joint Commission on Cancer [AJCC]2017 8^{th} edition). Patients were consented to an existing, institutional review board (IRB)-approved head and neck cancer tissue collection protocol (DF/HCC #09-472) prior to enrollment and sample acquisition. Clinical information and demographics were recorded for each participant along with initial treatment and response data. Patients were followed post-treatment to evaluate for biopsy-confirmed recurrence, survival status, and therapy-related toxicity. Recurrence was defined as locoregional tumor regrowth or distant metastatic spread (either or both).

Patient characteristics are shown in Table 5. The cohort comprised both male and female patients with a median age of 63. A majority of these patients were white non-Hispanic in origin. Approximately 50% of these patients had a history of alcohol consumption and/or smoking. The tumors analyzed in this study primarily originated either in the oropharynx (base of tongue or tonsil) or were collected from involved metastatic neck lymph nodes.

Consistent with the prognosis of HPV+ OPSCC, 4/19 (21%) patients within the cohort developed recurrence during a median follow-up time of 13.6 months (range: 4.2-29.1+) (Table 5).

TABLE 5
Patient characteristics

| | | | | Tumor | | ENE | Smoking | Alcohol | Prior |
|---|---|---|---|---|---|---|---|---|---|
| ID | Age | Sex | Ethnicity | site | Stage | (mm) | history | use | treatment |
| DFCI1 | 70 y | M | W-N | Base of | pT3N2-2 | >2 | Y | N | ChemoRT for |
| | | | | tongue | | | | | Bca, Chemo |
| | | | | | | | | | for CLL |
| DFCI2 | 64 y | M | W-N | Oropharynx | pT1N1-1 | N | Y | Y | No |
| DFCI3 | 63 y | M | W-N | Base of | cT1N1 | | N | Y | Basal cell |
| | | | | tongue | | | | | carcinoma |
| DFCI4 | 45 y | M | W-N | Tonsil | pT1N1-1 | N | N | Y | No |
| DFCI5 | 57 y | F | W-N | Tonsil | pT1N1-1 | >2 | N | N | No |
| DFCI6 | 55 y | M | B-N | Tonsil | pT1N1-1 | N | Y | Y | No |
| DFCI8 | 71 y | M | W-N | Base of | cTIN1M0 | | Y | Y | ChemoRT |
| | | | | tongue | | | | | for ThCa |
| DFCI9 | 85 y | F | W-N | Larynx | pT1N1-1 | N | Y | Rare | Prior RT |
| | | | | | | | | | for TNBC |
| DFCI11 | 32 y | M | W-N | Tonsil | pT1N1-1 | N | Y | Y | No |
| DFCI12 | 69 y | M | W-N | Tonsil | cT1N1- | | Y | Unknown | ChemoRT |
| | | | | | 3M0 | | | | (TestCA) |
| DFCI14 | 67 y | F | W-N | Met lymph | pT1N2-2 | >2 | Y | Unknown | No |
| | | | | node | | | | | |
| DFCI15 | 59 y | M | W-N | Tonsil | pT2N1-1 | N | N | Y | No |
| DFCI16 | 67 y | M | W-N | Tonsil | pT2N1-1 | >2 | N | N* | No |
| DFCI17 | 79 y | M | W-N | Met lymph | cT1N1M0 | | N | Rare | ChemoRT |
| | | | | node | | | | | |
| DFCI18 | 52 y | M | W-N | Tonsil | pT2N1-1 | >2 | Y | Y | No |
| DFCI19 | 54 y | F | W-H | Tonsil | pT0N1-1 | N | N | Y | No |
| DFCI20 | 51 y | M | W-N | Pharyngeal | pT2N0-1 | | N | N* | No |
| | | | | wall | | | | | |
| DFCI26 | 59 y | M | W-N | Tonsil | pT1N1-1 | N | N | Unknown | No |
| DFCI35 | 80 y | F | W-N | Lymph | pT0N1-1 | N | Y | Y (rarely) | Mohs surgery |
| | | | | Node | | | | | for basal cell |
| | | | | | | | | | carcinoma |

| | | Time to | Last | | HPV reads | | | Tumor |
|---|---|---|---|---|---|---|---|---|
| | Later | Recurrence | follow | HPV | (fragments/ | | Biopsy | HPV |
| ID | Recurrence | (months) | up | type | mL) | A02:01+ | GEX | GEX |
| DFCI1 | Y | 5 | 19.5 | 16 | 12971 | N | Y | Y |
| DFCI2 | N | | 19.5 | 16 | 344 | Y | Y | Y |
| DFCI3 | N | | 17.7 | 33 | 237 | Y | Y | Y |
| DFCI4 | Y | 13.6 | 19.4 | 16 | 10292 | N | Y | Y |
| DFCI5 | N | | 16.6 | 16 | 10 | N | Y | Y |
| DFCI6 | N | | 16.7 | 33 | 142 | Y | Y | Y |
| DFCI8 | N | | 15.8 | 16 | 935 | Y | N | N |
| DFCI9 | N | | 10.1 | 33 | 12 | Y | N | N |
| DFCI11 | N | | 14.9 | 16 | 43 | N | Y | Y |
| DFCI12 | N | | 17.7 | 16 | 3895.4 | N | N | N |
| DFCI14 | N | | 10.8 | 33 | 1702 | Y | Y | Y |
| DFCI15 | N | | 12.2 | 16 | 47 | Y | Y | Y |
| DFCI16 | N | | 13.6 | 16 | Indeterminate | N | Y | Y |
| DFCI17 | Y | 4.1 | 29.1 | 16 | Tissue | N | Y | N |
| | | | | | positive | | | |
| DFCI18 | N | | 10.2 | 16 | 21310 | N | Y | Y |
| DFCI19 | Y | 4.6 | 10.7 | 16 | 67 | N | N | N |
| DFCI20 | N | | 7.7 | 16 | 117 | Y | Y | Y |
| DFCI26 | N | | 7.4 | 33 | 12 | Y | N | N |
| DFCI35 | N | | 6.6 | 33 | 778 | N | Y | Y |

### Tissue Acquisition and Collection

Following informed consent, at the time of intra-office or interventional radiology facilitated core needle biopsy for diagnosis and/or transoral operative resection tissue specimens were prospectively collected. On the day of the procedure, freshly acquired tissue samples were submerged fully in pre-filled 1.5 mL cryovials or 5 mL Eppendorf tubes with MACS clear buffer and immediately labeled and stored at 4° C. until further processing.

### Blood and Buccal Swab Collection

On the same day as the planned biopsy or operative procedure for tissue procurement, patients underwent collection of 5×6 mL or 3×10 mL green top heparin (or EDTA purple top tubes based on availability) stored at ambient temperature and similarly shipped directly to Repertoire within 24 hours of venipuncture. Additionally, two buccal cheek swabs (RSC Buccal Swab DNA kit, Maxwell®, Madison, WI) were obtained from each participant pre-treatment with collection supervised by a study team researcher and stored at ambient temperature until further processing.

### HPV Plasma and Tissue Diagnostic Testing

Fine needle aspiration cytology, core needle, or surgical biopsy specimens were obtained from all patients to ensure clinical diagnostic accuracy. Immunostaining for p16 was performed in all cases and required 70% or great expression to be deemed positive, as is convention. Confirmatory tissue-based in situ hybridization (ISH) or polymerase chain reaction (PCR) testing was attempted in all cases to confirm HPV causality.

A platform test for HPV E6/7 transcripts of subtypes 16/18 by RNA ISH was performed, followed by reflex PCR to identify other high-risk subtypes if ISH returned negative. Results were interpreted by two independent anatomic pathologists with head and neck subspecialty training.

Baseline peripheral blood testing was performed pre-treatment on all participants for tumor tissue-modified virus (TTMV)-HPV DNA (NavDx®, Naveris, Waltham, MA) at the time of enrollment. This ultrasensitive digital droplet PCR assay detects the five most common high-risk subtypes of HPV (16, 18, 31, 33, and 35) and reports TTMV-HPV DNA fragments/mL of plasma (normalized across samples to generate a score) categorized as positive (>7 HPV16, >12 non-HPV16), negative (<5 fragments/mL), or indeterminate (5-7 HPV16, 5-12 non-HPV16) result for reporting purposes.

### Tumor Dissociation

Fresh tumor biopsy samples obtained from HPV-positive OPSCC patients were dissociated using a human tumor dissociation kit (Miltenyi Biotec, Bergisch Gladbach, Germany) per manufacturer guidelines. Briefly, tumors were weighed, and cut into small pieces (2-4 mm) in 1-2 ml RPMI-1640 media containing tumor dissociation enzymes. Tumor chunks were further minced on the gentleMACS Octo Dissociator (Miltenyi Biotec) at 370C for 15 minutes. Dissociated tumor cells were pelleted and re-suspended in 1-2 ml RPMI-1640 containing 10% human serum albumin (HSA). The cell suspension was filtered through a MACS SmartStrainer (70 m), pelleted, and res-suspended in 1 ml of RPMI-1640 with 10% serum. The dissociated tumor cells were counted, and viability was determined using the ViaStain viability dye (Nexcelom Bioscience LLC, Lawrence, MA, USA) using an automated cellometer (Nexcelom Bioscience LLC).

### HLA Typing

For HLA typing, gDNA was extracted from the peripheral blood of HPV-positive OPSCC patients by using GeneCatcher™ gDNA Blood Kit (Invitrogene). The quantity and quality of gDNA was evaluated in Nanodrop8000 (Thermofisher). gDNA was then amplified using LinkSēq™ HLA-ABCDRDQDP+384 Kit, and the dissociation profile was subjected to SureType software (One Lambda) to determine patient's HLA type. HLA typing was validated by buccal swab through a commercial service (Scisco Genetics, Inc.).

### Antigen Library Design

The amino acid sequences of HPV16 E1, E2, E4, E5, E6, and E7 were run through the program NetMHCPan in order to predict all possible 9-mers (class I) or 15-mers (class II) binding to their respective MHC complexes with an affinity of 500 nM or stronger. HPV peptides for A*01:01, A*02:01, A*03:01, A*24:02, B*07:02, B*08:01, DRB1*03:01, DRB1*07:01 and DRB1*15:01 were predicted. A set of well-defined viral epitopes from Cytomegalovirus, Epstein-Barr virus, Influenza viruses (CEF peptide pool) and SARS-CβV2 that elicit T cell responses in the population at large was added to the library at the end. Antigenic peptides with 500 nM affinity or lower were then selected for inclusion.

Production of pMHC Library Pools

Class I MHC extracellular domains were expressed in E. coli and refolded along with beta-2-microglobulin and ultraviolet (UV)-labile place-holder peptides. A C-terminal sortase recognition sequence on the HLA was modified by sortase transpeptidation with a synthetic alkynylated linker peptide, featuring an N-terminal triglycine connected to propargylglycine via a PEG linker (Genscript, Piscataway, NJ). The modified HLA monomer was then purified by size exclusion chromatography (SEC). Full-length streptavidin with an N-terminal Flag tag and a C-terminal sortase recognition sequence and 6xHisTag was prepared by expression and purification from E. coli using immobilized metal affinity chromatography and SEC. Streptavidin was modified by sortase transpeptidation with a synthetic azidylated linker peptide, featuring an N-terminal triglycine connected to picolyl azide via a PEG linker (Click Chemistry Tools, Scottsdale, AZ). HLA tetramers were produced by mixing alkynylated HLA monomers and azidylated streptavidin in 0.5 mM copper sulfate, 2.5 mM BTTAA (2-(4-((Bis((1-(tert-butyl)-1H-1,2,3-triazol-4-yl)methyl)amino)methyl)-1H-1,2,3-triazol-1-yl)acetic acid) and 5 mM ascorbic acid for up to 4 h on ice, followed by purification of highly multimeric fractions by SEC. Individual peptide exchange reactions containing 500 nM HLA tetramer and 60 mM peptide were exposed to long-wave UV (366 nm) at a distance of 2-5 cm for 30 min at 4° C., followed by 30 min incubation at 30° C. A biotinylated oligonucleotide barcode (Integrated DNA Technologies) was added to each individual reaction followed by 30 minute incubation at 4° C. Individual tetramer reactions were then pooled and concentrated using 30 kDa molecular weight cut-off centrifugal filter units (Amicon). Tetramer production was quality controlled using SEC, sodium dodecyl sulfate polyacrylamide gel electrophoresis (SDS-PAGE), and UV-mediated peptide exchange by assessing binding to peptide-expanded cell lines.

The MHC Class II α- and β-chain extracellular domains were recombinantly expressed with C-terminal Myc and His tag sequences, respectively. For DRB1*15:01 the Myc tag was replaced with a V5 tag. The N-terminus of the β-chain was fused to CLIP peptide followed by a flexible Factor Xa-cleavable linker. Both α- and β-chains were co-expressed in CHO cells and secreted into the expression medium as a stable CLIP-loaded heterodimer. Heterodimerization of the α- and β-chains of DRB1*07:01 and DRB1*1501 was forced using a fusion of an engineered human IgG1-Fc protein to each chain, but no additional heterodimerization motif was used for DRB1*11:01. Following CHO expression, the heterodimer was purified by immobilized metal ion affinity chromatography and size exclusion chromatography (SEC) and was subjected to an over-night biotinylation reaction at 4° C. a using a commercial BirA biotin-protein ligase reaction kit (Avidity, LLC). Biotinylated proteins were further purified via SEC and digested with Factor Xa protease according to the manufacturer's instructions (New England BioLabs). The proteolytic reaction was stopped by the addition Factor Xa inhibitor, 1,5-Dansyl-Glu-Gly-Arg Chloromethyl Ketone, Dihydrochloride (Sigma Aldrich). Class II proteins were concentrated to ~30 μM and stored at −80° C. prior to preparation of the corresponding barcoded peptide libraries. Individual peptide exchange reactions containing 7 uM Class II heterodimer and 350 uM peptide in a sodium citrate buffer (1% mM sodium citrate, 1% mM sodium chloride, 0.1% octyl glucoside, SIGMAFAST™ Protease Inhibitor Cocktail Tablets) were incubated at pH5.5 and 30° C. overnight. The individual reactions were then neutralized using 1M Tris-HCL at pH10 to a final concentration of 0.14M Tris-HCL. In a separate container, Klickmers (Immudex, Denmark) were individually barcoded using biotinylated oligonucleotide barcodes (Integrated DNA Technologies) and incubated for 30 minutes at 4° C. Individually exchanged Class II heterodimers were then added to the individually barcoded Klickmers and allowed to incubate for 30 minutes at 4° C. 2 mM D-Biotin (Invitrogen) was then added to each individual reaction to quench the streptavidin, and allowed to incubate for 30 minutes at 4° C. These individual reactions were then pooled and concentrated using 30 kDa molecular weight cut-off centrifugal filter units (Amicon), and subsequently washed using cold PBS (Gibco) until a desired number of washes was achieved. The pool was then concentrated to the desired final concentration for staining.

### Cell Staining

Dissociated tumor cells obtained from the OPSCC patients were subjected to magnetic-activated cell sorting (MACS) to get an enriched CD3+, CD8+ or CD4+ T cells using CD3+ T cell or CD8+ T cell isolation kit respectively (Miltenyi Biotec) following the manufacturer's protocol. The enriched fraction of T cells was then stained with 1 nM final concentration tetramer library in the presence of 2 mg/mL salmon sperm DNA in PBS with 0.5% BSA solution for 20 minutes. Cells were then labeled with anti-TCR antibody-derived tag (ADT, IP26, Biolegend, San Diego, CA, USA) for 15 minutes followed by washing. Tetramer bound cells were then labeled with phycoerythrin (PE) conjugated anti-DKDDDDK-Flag antibody (BioLegend) followed by dead cell discrimination using 7-amino-actinomycin D (7-AAD). The live, tetramer positive cells were sorted using a Sony MA9% Sorter (Sony Biotechnology, San Jose, CA, USA). Final counts were determined and viability was assessed using an automated cell counter (Nexcelom Bioscience LLC) for use in single-cell encapsulations.

### Sample Multiplexing

To ensure sufficient cDNA production in single-cell sequencing, sample multiplexing was used for several experiments. When applied, samples were stained and sorted separately according to the protocol described above using anti-TCR ADTs with unique barcodes. Labeled samples were combined prior to encapsulation and single-cell sequencing.

### Single-Cell Sequencing

Single-cell encapsulations were generated utilizing 5′ v1 Gem beads from 10× Genomics (Pleasanton, CA, USA) on a 10× Chromium controller and downstream TCR, and Surface marker libraries were made following manufacturer recommended conditions. All libraries were quantified on a BioRad CFX 384 (Hercules, CA, USA) using Kapa Biosystems (Wilmington, MA, USA) library quantified kits and pooled at an equimolar ratio. TCRs, surface markers, and tetramer generated libraries were sequenced on Illumina (San Diego, CA, USA) NextSeq550 instruments. Sequencing data were processed using the Cell Ranger Software Suite (Version 3). Samples were demultiplexed and unique molecular identifier (UMI) counts were quantified for TCRs, tetramers, and gene expression.

### Single-Cell Transcriptomic Analysis

Hydrogel-based RNA-seq data were analyzed using the Cell Ranger package from 10× Genomics (v3.1.0) with the GRCh38 human expression reference (v3.0.0). Except where noted, the Seurat R package (v1.6.0) was used to perform the subsequent single cell analyses. Any exogenous control cells identified by TCR clonotype were removed before further gene expression processing. Hydrogels that contain UMIs for less than 3% genes were excluded. Genes that were detected in less than 3 cells were also excluded from further analysis. Several additional quality control thresholds were also enforced. To remove data generated from cells likely to be damaged, upper thresholds were set for percent UMIs arising from mitochondrial genes (10%). To exclude data likely arising from multiple cells captured in a single drop, upper thresholds were set for total UMI counts based on individual distributions from each encapsulation (from 1500 to 3000 UMIs). Any hydrogel outside of any of the thresholds was omitted from further analysis. A total of 133,182 hydrogels were carried forward. Gene expression data were normalized to counts per 10,000 UMIs per cell (CB10K) followed by logip transformation: ln(CB10K+1).

Highly variable genes were identified (total of 1,567 genes) and scaled to have a mean of zero and unit variance. They were then provided to Seurat (v3.0)) to perform batch integration and dimension reduction. The data were then used to generate shared principal components which was in turn used to generate a UMAP representation. Shared neighbor graphs and Leiden clustering were subsequently performed. The hydrogel data (not scaled to mean zero, unit variance, and before extraction of highly variable genes) were assigned labels using SingleR (v1.4.0) using the Human Primary Cell Atlas Data reference from Celldex (v1.0.0). SingleR was used to annotate the clusters with their best-fit match from the cell types in the references. Clusters that yielded cell types other than types of the T Cell lineage were removed from consideration and the process was repeated starting from the batch integration step. The best-fit annotations from SingleR after the second round of clustering and the annotation was assigned as putative labels for each Leiden cluster. Further clustering of transcriptomic data was performed across the genes using KMeans in sklearn (v0.24) with n_clusters set to 8. As the method has a preference to assign like-sized clusters, further consolidation of two central memory clusters was performed.

To provide corroboration for the SingleR best-fit annotations and further evidence as to the phenotype of the clusters, gene panels representing functional categories (Naive, Effector, Memory, Exhaustion, Proliferation) were used to score each hydrogel's expression profiles using scanpy's “score_genes” function which compares the mean expression values of the target gene set against a larger set of randomly chosen genes that represent background expression levels. The gene panels for each class were: Naïve—TCF7, LEF1, CCR7; Effector—GZMB, PRF1, GNLY; Memory—AQP3, CD69, GZMK; Exhaustion—PDCD1, TIGIT, LAG3; Proliferation —MKI67, TYMS. The gene expression matrix for all hydrogels were first imputed using the MAGIC algorithm (v2.0.4). These functional scores were the only data generated from imputed expression values.

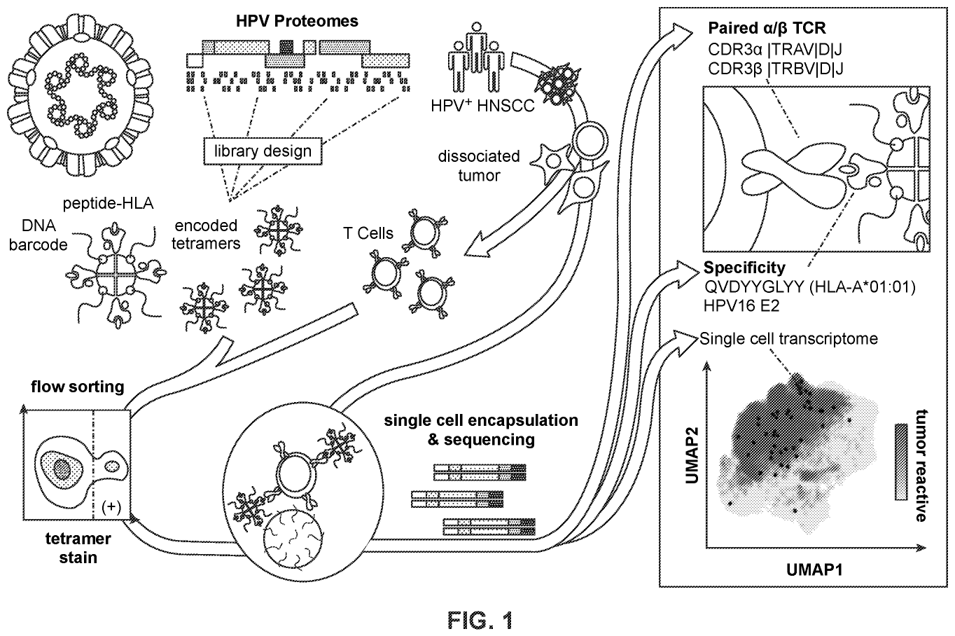

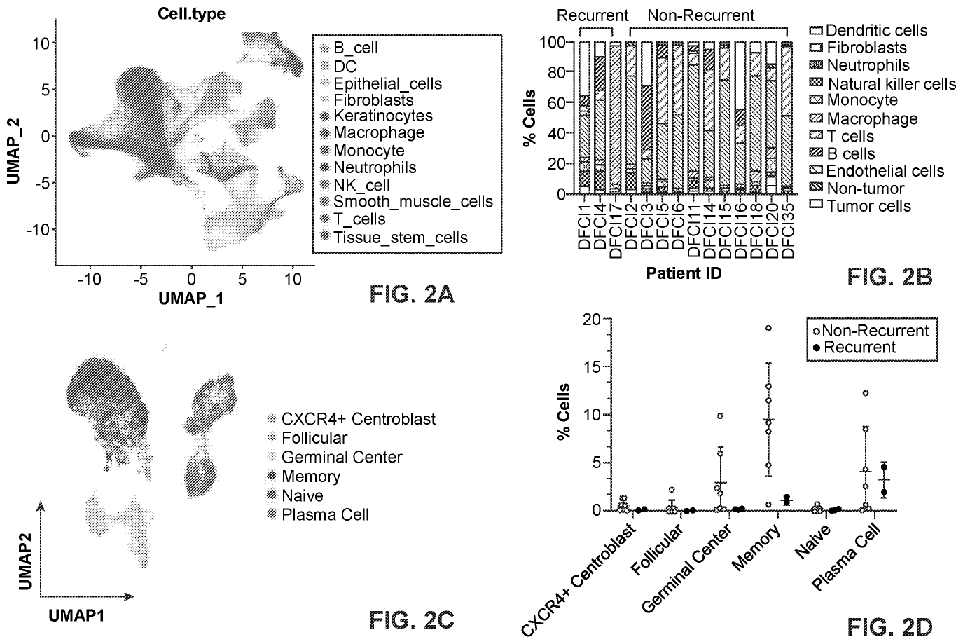

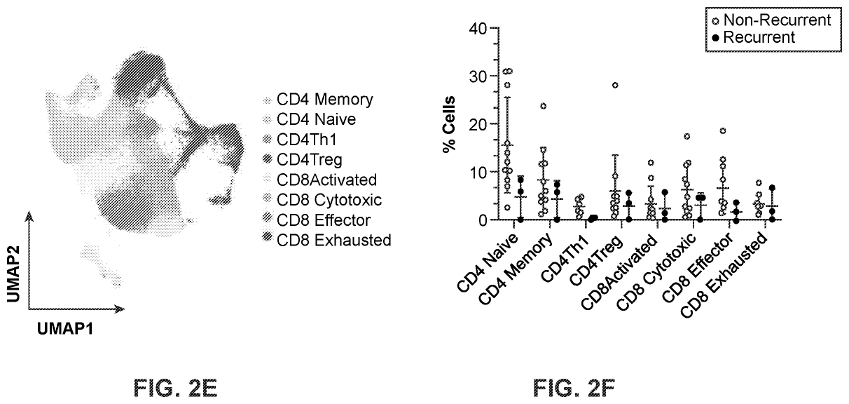

The results of the RNAseq from the HPV16-associated tumors (n=14) show that the tumor microenvironment (TME) was composed of a complex mix of cell types aside from tumor, T-cells, and B cells (FIG. 1). T cells from these HPV-associated tumors were further re-clustered (FIG. 2E) to discern different T cell sub-populations (FIG. 2F). Similarly, differential composition of B cells is displayed following re-clustering analysis from 13 different tumors where B-cells were detected (DFCI17 had no B-cells). B cells from these HPV-associated tumors were further re-clustered (FIG. 2C) to discern different T cell sub-populations (FIG. 2D).

### HPV Calling

A transcriptome reference was built including different strains from HPV (hpv16, hpv18, hpv31, hpv33, hpv45, hpv52 and hpv 58). Fastq files from 10× single cell data were pseudo-aligned to this reference using Kallisto v0.46.0. BUStools v0.39.4 was used to error correct barcodes, collapse UMIs, and produce gene count matrices to use for further analysis. HPV counts were log transformed and normalized to tumor content across patients to compare relative expression levels.

### Scoring Peptide-HLA-TCR Interactions

Tetramer data analysis was performed using Python (v3.7.3). For each single-cell encapsulation, tetramer UMI counts (columns) were matrixed by cell (rows) and log-transformed. Duplicates of this matrix were independently Z-score transformed by row or column, and subsequently median-centered by the opposite axis (column or row), respectively. For each peptide-LA-cell interaction, this provided two scores—inter-tetramer ( [symbol] ) and inter-cell ( [symbol] ), which were used to calculate a classifier for unique CDR3 a/b clonotypes across N cells as N×Z̅ₜₑₜ× [symbol] Classifier thresholds for positive interactions were set at 40, 36, 50, and 65 for A*02:01, B*07:02, A*24:02, and A*01:01, respectively.

### TCR Network Analysis

TCR motif analysis was performed in python (v3.8.5) using TCRdist3 (v0.2.2) on both alpha and beta chains. Network graphs were assembled using networkx (v2.7.1). Nodes representing alpha (circle) and beta chains (square) were connected by edges to each other within a clonotype and to corresponding chains from other cells when distances were less than or equal to 11 and 23, respectively. Graphs were drawn using a spring layout with spring weights inversely proportional to distances. Once clusters were identified, sequence alignment was performed using the pairwise2 module in Biopython (v1.78) and visualized using logomaker (v0.8).

### Example 2. Profiling of HPV16+ OPSCC Tumors at Single-Cell Resolution

This example describes the profiling of HPV+ OPSCC tumors at single-cell resolution by analysis of cell phenotype via single-cell transcriptomics, expression of HPV genes, and paired TCRα/TCRβ sequences of tumor infiltrating T cells.

Briefly, for each patient sample, an ex vivo characterization of cell phenotype via single-cell transcriptomics, expression of HPV genes, and paired TCRα/TCRβ sequences of tumor infiltrating T cells was performed (an overview of the process is shown in FIG. 1).

Samples from HPV16-associated OPSCC tumors (n=14) were analyzed using cell type inference from single cell RNAseq data of dissociated tumors. Results were plotted as uMAP clusters and as % cells. The results show that the tumor microenvironment (TME) was composed of a complex mix of cell types aside from tumor, T-cells, and B cells (FIG. 2A). HPV⁺ patients show heterogenous distribution of immune and non-immune cell types in the dissociated tumors from recurrent vs non-recurrent patients (FIG. 2B).

There were no significant differences by recurrence status between cell types. One patient who recurred had an extremely high proportion of macrophages, but this was not representative of the other two patients that recurred (FIG. 2B)

T cells from these HPV-associated tumors were further re-clustered (FIG. 2E) to discern different T cell sub-populations (FIG. 2F). Similarly, differential composition of B cells is displayed following re-clustering analysis from 13 different tumors where B-cells were detected (DFCI17 had no B-cells). B cells from these HPV-associated tumors were further re-clustered (FIG. 2C) to discern different T cell sub-populations (FIG. 2D).

### Example 3. Correlation of HPV Gene Expression with Clinical Variables and T Cell Subsets

This example describes the correlation of HPV16 and HPV33 gene expression with clinical variables and T cell subsets.

HPV gene expression patterns in tumors can vary and this can occasionally be associated with loss of expression of E1, E2, and E5 and lower overall HPV expression. It has also been reported that patients with higher expression of E2, E4, and E5 have better outcomes compared to those where E6 and E7 expression predominates. To determine how tumor HPV gene expression within these untreated specimens may relate to patient outcomes including tumors associated with either HPV16 or HPV33 tumor samples were tested for HPV protein expression.

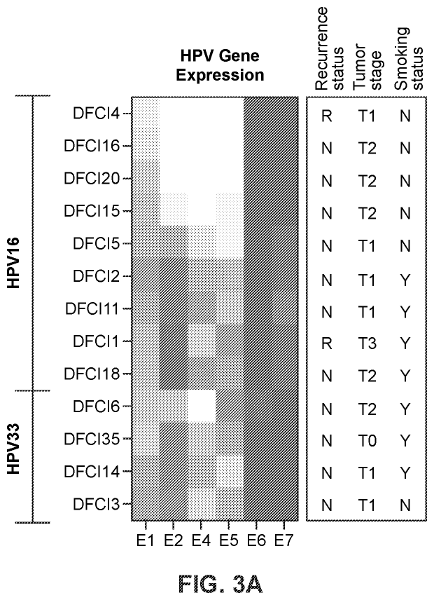

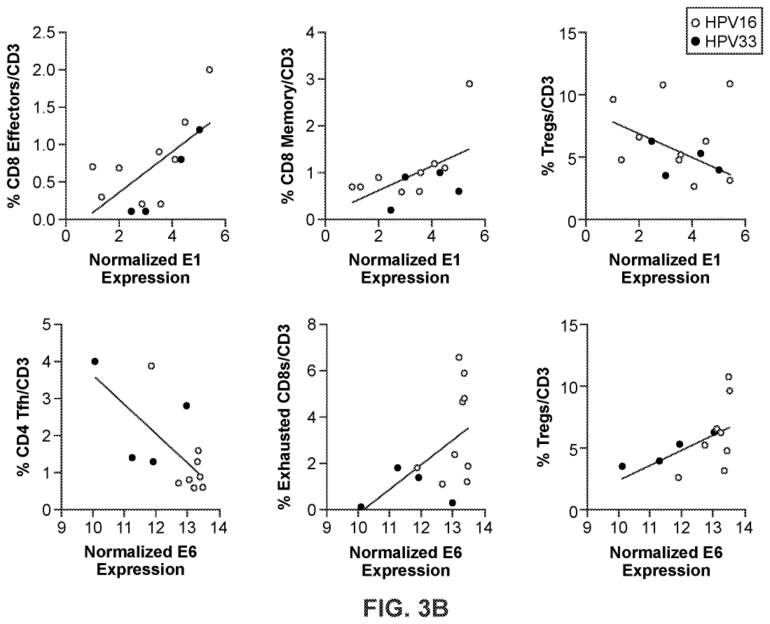

Samples from patients with more advanced tumor (T0, T1, T2, and T3) stage or those who had recurrent disease were analyzed for expression of E1, E2, E4, and E5. There was no association between HPV gene expression pattern and tumor size, nodal status (number of involved nodes) or smoking status in a subgroup of 13 patients (FIG. 3A). The patient with high macrophages had no detectable tumor cells and therefore was excluded from this analysis. Among the other two patients who recurred, one of them showed the expected E6/E7 expression only, but the other had expression of E1, E2, E4 and E5 with particularly high expression of E2. HPV33+ patients had a different distribution of HPV gene expression compared to HPV16. All four HPV33+ patients maintained expression of E1, E2, E4, and E5 in addition to the expected E6 and E7.

FIG. 3A shows the relationship between HPV gene expression and tumor stage, recurrence status, or smoking status. Significant positive correlation was present between HPV E1 gene expression and CD8 effector (FIG. 3B), and CD8 memory T cells whereas negative correlation was present between E1 gene expression and Tregs. CD4 Tregs as defined by FOXP3 expression were detected, and there was a trend towards negative association with E1 gene expression. The correlations between HPV E6 gene expression and T cell subsets was analyzed. A Pearson's correlation analysis was run and it shows significant negative association between HPV E6 gene expression and CD4 Tfh cells but CD8 exhausted and Tregs show a reverse trend. Gene expression signatures associated with each of the T cell subsets is shown. Together, the results indicate that higher HPV E1 gene expression results in a more robust proinflammatory TME associated with greater CD8 infiltration, activation and differentiation in virally-driven OPSCC.

### Example 4. Analysis of Cytotoxic T-Cell Responses in Treatment-Naive Oropharyngeal Squamous Cell Carcinoma Patients

This example describes the analysis of cytotoxic T-cell responses to HPV antigens in treatment-naïve oropharyngeal squamous cell carcinoma patients.

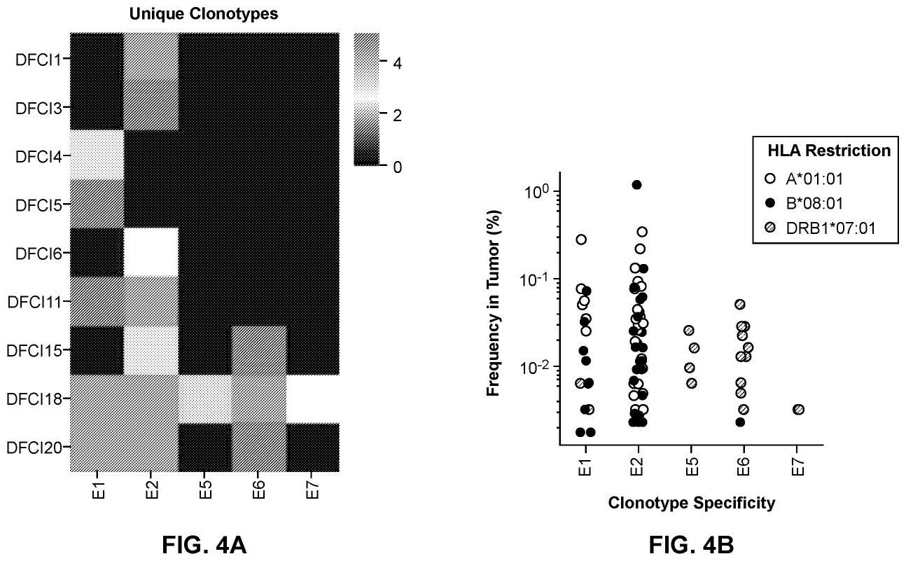

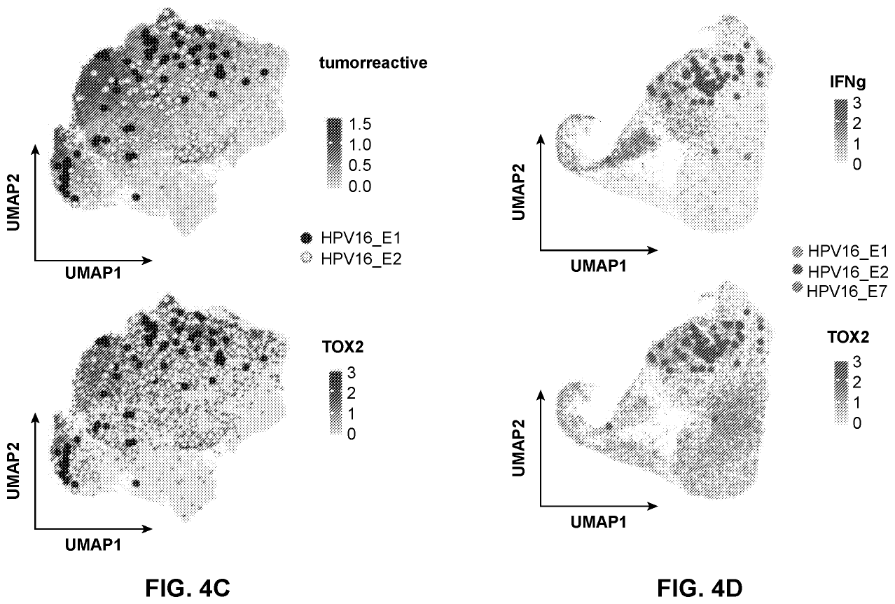

Briefly, T-cells from five HPV16+ tumors were probed with tetramers from HIPV16 E1, E2, E4, E5, E6, and E7 and three with tetramers from E6 and E7 alone. One HPV16+ tumor was not stained due to limited cell numbers. The analysis shows reactivity to proteins E1, E2, E5, E6 and E7 and no reactivity to E4 (FIG. 4A). T-cells reacting to E1 and E2 were more abundant than T-cells reacting to E5, E6, E7. Reactivity to proteins E1 and E2 in was found particularly in patients with HLA-A*01:01 and HLA-B*08:01. T-cell reactivity to the epitope QVDYYGLYY-A*01:01 from HPV16 E2 was previously reported by Eberhardt et al. from an HPV+ OPSCC patient. T-cells specific for this epitope made up approximately 1.6% of TILs in patient DFCI18 (FIG. 4B).

T cells were further analyzed by single cell gene expression (FIGS. 4C and 4D). HPV-reactive CD8s recognized E1 and E2 and showed moderate expression of TOX2 indicative of exhaustion and high expression of genes associated with tumor reactivity (FIG. 4C). Most HPV-reactive CD4s recognized E5, E6 and E7 and showed moderate to low expression of IFN-γ, and moderate to high expression of TOX2 indicating these clonotypes may be more exhausted and less able to carry out cytotoxic gene programs (FIG. 4D).

Analysis of gene expression differences in HPV16+ versus HPV33+ patients

This example describes the analysis of gene expression differences in HPV16+ versus HPV33+ patients.

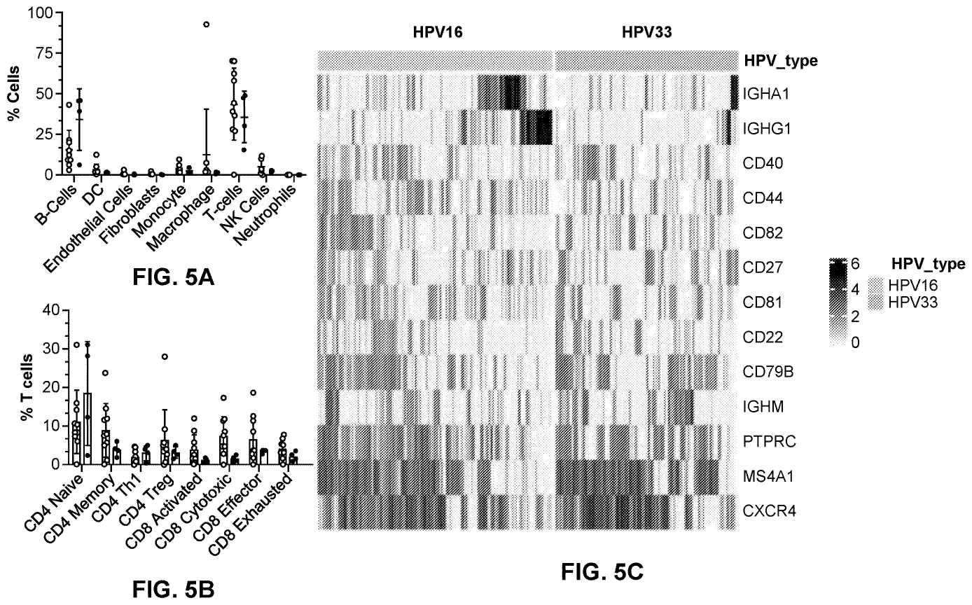

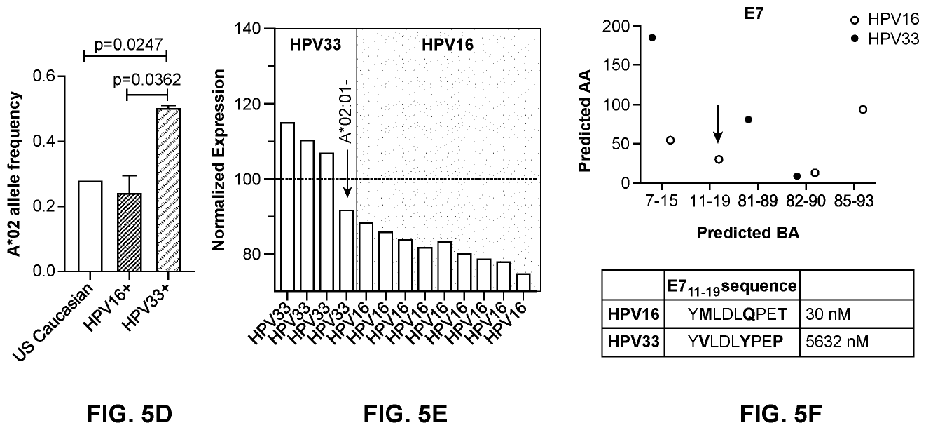

HPV strain-specific differences in proportion of different immune cells (FIG. 5A) and T cell subsets (FIG. 5B) were analyzed. No differences in DCs, fibroblasts, monocytes, macrophages, NK cells, neutrophils or overall T-cell number between HPV16+ and HPV33+ patients were detected (FIG. 5A). A significantly higher proportion of B-cells in HPV33+ versus HPV16+ patients (p=0.0336) was observed. Cytotoxic T-cells made up 1.06% of dissociated tumor cells in HPV33+ patients compared to 2.71% of HPV16⁺ patients (p=0.2203) (FIG. 5B). Fewer exhausted T-cells (p=0.1026) and overall CD8s (p=0.0945) in HPV33+ compared to HPV16. There were no differences in CD8 memory cells, total CD4s, Tregs, Th1, Th2, Th17 or Tfh between strains. The CD8+ T cells showed a trend towards increased presence among HPV16+ tumors compared to the HPV33+ tumors. Further phenotypic profiling on different B-cell subsets revealed that compared to the HPV16+ tumors, the HPV33+ cohort showed reduced expression of genes that are representative of B-cell differentiation and expansion into high-affinity antibody-secreting plasma cells such as IGHG1 and IGHA1, a subpopulation of B-cells important in the induction of long-term serological immunity. IGHA1 and IGHG1, genes associated with B-cell expansion and differentiation, were the top differentially expressed genes in B-cells from patients with HPV33 versus HPV16-driven tumors (FIG. 5C). Elevated expression of CD40, CD82, and CD44 that play a role in antigen presentation, co-stimulation, and immune activation pathways among HPV16+ tumors was found. In summary, the analysis shows strain-dependent phenotypic differences in T-cells and B-cells that could help explain prior reports of worse outcomes in HPV33+ OPSCC.

The HLA-A*02 allele distribution was compared across the patient cohort [total n=13 (HPV16+); n=6 (HPV33+)] with the US Caucasian population. The HLA-A*02 allele frequency was significantly higher among HPV33+ patients in the cohort compared to the US Caucasian population [A*02 allele frequency: 0.5 (DFCI dataset) compared to 0.28 (US Caucasian)](FIG. 5D) (Source: www.allelefrequencies.net). The data in the patient cohort was concordant with an independent dataset of OPSCC patients (TEMPUS, Inc) where total n=16 HPV33+ and n=202 HPV16+ patients were probed for these analyses. The results show 1.77-fold more HPV33+ patients carrying the HLA-A*02 allele than in US Caucasians (p=0.0247).

The epitopes well presented in HPV16 but missing in HPV33 which could potentially explain susceptibility to HPV33 but not HPV16-driven OPSCC among HLA-A*02:01+ patients were determined. Single cell HPV gene expression was determined in tumor cells from 4 patients with HPV33 and 9 with HPV16. Two HPV33+ patients in the cohort had no tumor GEX data. Comparing the ratios of E7/E6 expression across this cohort, HLA-A*02:01+ HPV33+ patients had higher E7 gene expression than E6 (FIG. 5E). Comparative analysis shows higher E7:E6 gene expression in A*02:01 HPV33+ versus HPV16+ or HPV33+A*02:01—patient tumors.

To test if there was a recognition deficit in A*02:01 of HPV33 E7 versus HPV16 the program NetMHCpan I was used to predict potentially presented epitopes from HPV16 versus HPV33 HLA-A*02:01. While HPV16 and HPV33 are similar in sequence, they are not identical and differences in their amino acid sequence results in different predicted epitopes for many regions of the HPV genome (FIG. 5F). However, E7 epitope prediction revealed that each strain had 4 predicted epitopes within E7 with sequence differences between these epitopes. One epitope predicted to have high affinity in HPV16 E7—amino acids 11-19, was not predicted to be presented in HPV33 (FIG. 5F). HPV16 E711-19 has been reported to be a critical epitope in HPV16 recognition and has been the target of several clinical trials seeking to treat HPV16-driven malignancies. Therefore, a deficiency in HPV E7 recognition by T-cells in HPV33 HLA-A*02:01⁺ may pose an elevated risk of virally-driven malignancy for this HPV subtype. Data in figures FIG. 5A, B, D are shown as mean±SEM. Statistical significance was analyzed by performing Student's t-test. p<0.05 is considered significant.

### Example 5. E2-Reactive Public and Pseudo-Public Clonotypes Validate in Functional Assay

It has been reported that paired CDR3 TCRα and TCRβ amino acid sequences can be shared across individuals, and similar TCR sequences may recognize the same epitope. TCR homology across patients with matched HLAs was analyzed for similar or identical TCRs recognizing the same antigen to infer T-cell reactivity. Dissociated tumor cells (DTCs) from all patients could not be analyzed with tetramer assay due to resection size limitations or tetramer availability at the time of collection. TCR sequences were determined by single cell sequencing data and T cells with native TCRs from dissociated tumor cells or recombinantly expressed TCRs were analyzed by tetramer assay.

TCR sequences were aggregated across single cell sequencing data of patient tumor resections (n=14) and assessed for homology of TCR alphas and TCR betas using the program TCRDist3. Tetramer-identified TCRs were cross-mapped to infer specificity onto TCR clusters.

A level of TCR alpha and TCR beta sharing homology at the amino acid and gene level across these patients particularly in T-cells reacting to viral antigens, with identical matches or encoded sequences differing by only 1-2aa was observed.

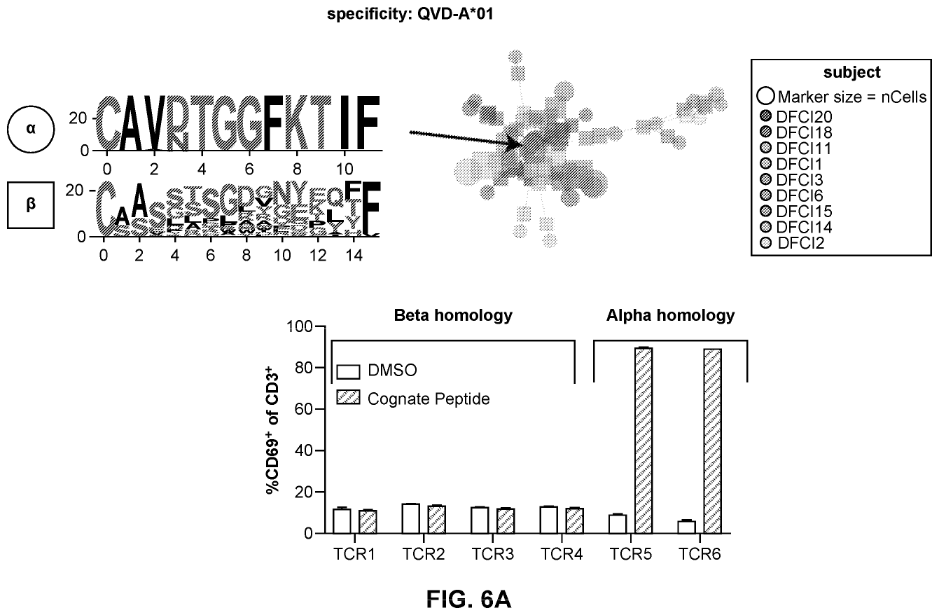

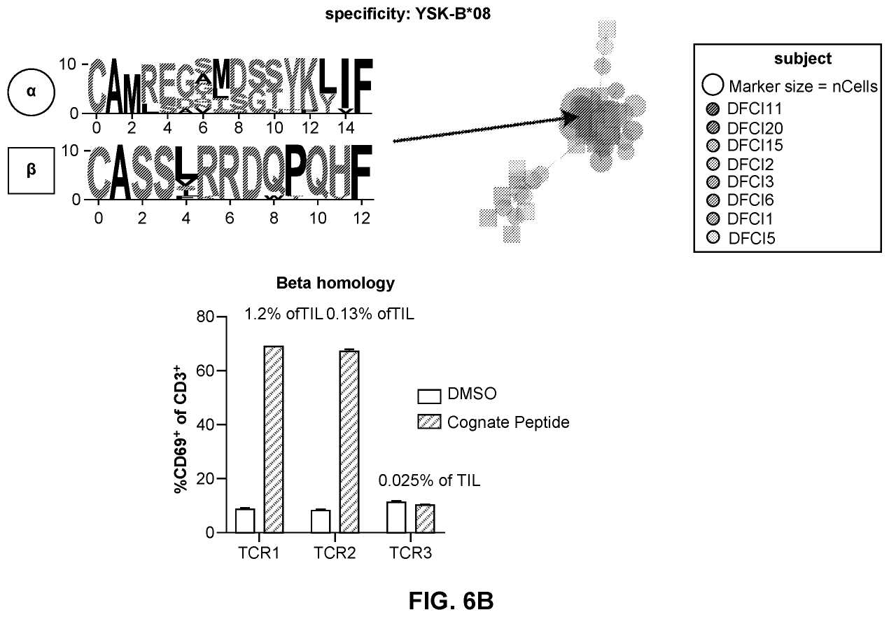

Public TCR clonotypes responding to QVDYYGLYY-A*01:01 were analyzed for TCRα and TCRβ sequences. HPV16 E2 QVDYYGLYY-A*01:01—specific T-cells showed strong clonotype sharing across patients (FIG. 6A), primarily from patients DFCI1, DFCI11, DFCI20, DFCI18 (FIG. 6A), with a common TCR CDR3 alpha motif. T-cells from several different patients appearing in this cluster, with heaviest contributions from DFCI1, DFCI11, DFCI20 and DFCI18, all patients that were A*01:01⁺ and HPV16⁺ (FIG. 6A).

TCRs binding to the QVDYYGLYY-A*01:01 (HPV16 E2) tetramer sharing the TCR CDR3 alpha CAVDTGGFKTIF or CAVNTGGFKTIF motif were identified between patients DFCI18 and DFCI20. To test the validity of these TCR-epitope interactions, recombinant TCRs were purchased and expressed in J76-CD8-NFAT-GFP cells, incubated overnight with cognate peptide and HLA-matched antigen presenting cells.

TCR clonotypes responding to HPV16 E2 YSKNKVWEV were analyzed for TCR CDR3 sequences. HPV16 E2 YSKNKVWEV-B*08:01—specific T-cells showed strong clonotype sharing across patients (FIG. 6B). T-cells in this cluster primarily originated only from patients who were A*01:01⁺ and HPV16⁺ and were united by a common TCR CDR3 beta motif (FIG. 6B).

(FIG. 6B). From this network 3 clonotypes were selected to test in the TCR-epitope validation assay from patient DFCI11, whose TILs were unable to be probed directly with tetramer. Two out of three of these clonotypes validated, including those constituting 1.2% and 0.13% of that patient's TIL (FIG. 6B). Two of three TCRs derived from tumor of patient DFCI11 presented with beta chain homology signal when presented with cognate peptide and made up 1.2% and 0.13% of TIL. Data represents mean±SD.

### Example 6. Validation of Additional TCR-Epitope Pairs

### Recombinant TCR Validation

TCR sequences identified from patient samples were cloned in the pLVX-EFla lentiviral backbone (Takara) as a bicistronic TCRB-T2A-TCRα vector. Viral supernatants from transfected HEK 293T cells were collected 48 and 72 hours after transfection (MIR6655 TransIT Lentivirus System) and added to the parental TCRα/β^{-/-} Jurkat J76 cell line (ref #) expressing CD8 and a nuclear factor of activated T cells (NFAT)-green fluorescent protein (GFP) reporter, referred to as J76-CD8-NFAT-GFP. Recombinant TCR surface expression was confirmed through flow cytometry by staining transduced J76-CD8-NFAT-GFP cells with anti-CD3—PE (Clone UCHT1) and anti-TCRα/β-allophycocyanin (APC) antibodies (Clone IP26).

To assess functional activity of recombinant TCRs, J76—CD8-NFAT-GFP expressing recombinant TCRs were incubated at a 1:1 ratio with the HLA-A*01:01⁺, HLA-B*07:02⁺ or DRB*15:01 expressing B-LCL cell line, with a final concentration of 0.5% dimethylsulfoxide (DMSO, vehicle) or 5 μM of cognate peptide (Vivitide, >95% pure). Cell mixtures were incubated at 37° C., 5% C02. At 16 hours, cells were washed and blocked with staining buffer (BD 554656), stained with anti-CD3-PE-Cy7 (Clone UCHT1) and anti-CD69-APC (Clone FN50) antibodies, and analyzed using the Sartorius iQue Screener Plus and FlowJo v10. CD69 activity was measured as percent positive of CD3⁺ cells.

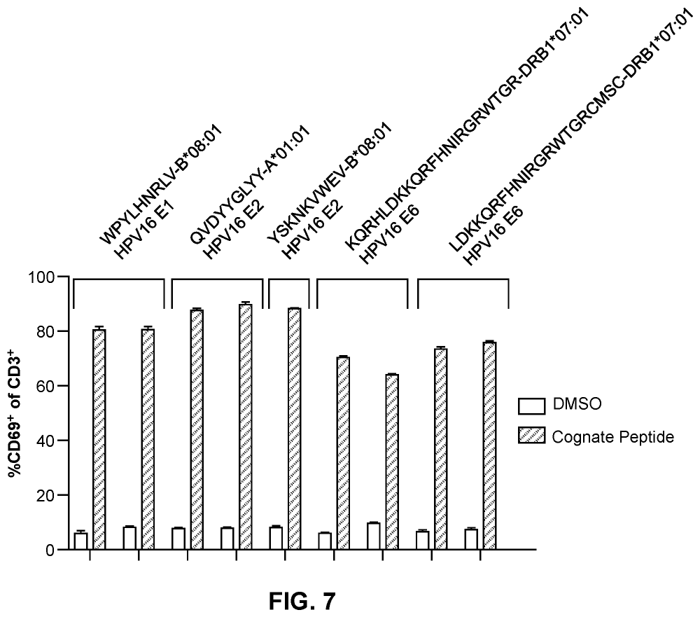

Activation was assessed by NFAT and CD69 signal by flow cytometry (FIG. 7). Each of these TCR CDR3 alphas was tested in the J76 signaling assay with one of its paired beta chains and confirmed the validity of this interaction. TCRs with beta chains homologous to QVDYYGLYY-reactive T-cells failed to signal but both TCRs with similar alpha chains signal when presented with cognate peptide (FIG. 6A). Two out of three TCRs with beta chains homologous to YSKNKVWEV-reactive T-cells signal when presented with cognate peptide (FIG. 6B). Data represents mean±SD.

### External Dataset

Records of 250 HPV⁺ OPSCC patients were obtained from TEMPUS, Inc. HLA class I typing for each sample was performed using Optitype to 4-digit resolution on DNA-seq data. HPV typing on each sample was performed using RNA-Seq data using Kallisto-DESeq2 workflow. Kallisto was used for quantifying abundances of HPV strain specific transcripts followed by count normalization using DESeq2 R package. HPV strains included HPV16, HPV18, HPV31, HPV33, and HPV45. Genes included E1, E2, E4, E5, E6, E7, L1 and L2 specific to each strain. HPV strain was assigned based on detection of strain specific HPV E* genes. Specifically, for a given sample at least one E* gene for that strain must have expression greater than/equal to 0.75 units.

### Statistical Analysis

Detailed description on data processing, normalization, and handling is included in each of the methods' sub-sections. Statistical analysis was performed using GraphPad Prism 9 software. Statistical analyses of differences were performed using two-sided Student's t-test, or Wald test wherever applicable. Pearson's correlation analysis was run to determine association between HPV gene expression and different cell types.

### INCORPORATION BY REFERENCE

All publications and patents cited throughout the text of this specification (including all patents, patent applications, scientific publications, manufacturer's specifications, instructions, etc.) are hereby incorporated by reference in their entirety for all purposes. To the extent the material incorporated by reference contradicts or is inconsistent with this specification, the specification will supersede any such material.

### EQUIVALENTS

The invention may be embodied in other specific forms without departing from the spirit or essential characteristics thereof. The foregoing embodiments are therefore to be considered in all respects illustrative rather than limiting the invention described herein. The scope of the invention is thus indicated by the appended claims rather than by the foregoing description, and all changes that come within the meaning and range of equivalency of the claims are intended to be embraced therein.

What is claimed is:

1. A T cell receptor (TCR) comprising an alpha chain variable domain (Vα) and a beta chain variable domain (Vβ), wherein the Vα comprises a CDR3 sequence of SEQ ID NO: 3, 5, 7, 9, 11, 13, 15, 17, 19, 21, 23, 25, 27, or 29, and the Vβ comprises a CDR3 sequence of SEQ ID NO: 2, wherein the TCR binds an epitope comprising the amino acid sequence of YSKNKVWEV (SEQ ID NO: 1) presented by HLA-B*08:01.

2. The TCR of claim 1, wherein the CDR3 sequence in the Vβ comprises the amino acid sequence of SEQ ID NO: 4, 6, 8, 10, 12, 14, 16, 18, 20, 22, 24, 26, 28, or 30.

3. The TCR of claim 1 or 2, wherein the Vβ further comprises the CDR1 and CDR2 amino acid sequences of TRBV7-6.

4. The TCR of claim 1, 2, or 3, wherein the Vβ comprises an amino acid sequence at least 90% identical to the amino acid sequence of TRBV7-6.

5. The TCR of claim 1 or 2, wherein the Vβ further comprises the CDR1 and CDR2 amino acid sequences of TRBV7-7.

6. The TCR of claim 1, 2, or 5, wherein the Vβ comprises an amino acid sequence at least 90% identical to the amino acid sequence of TRBV7-7.

7. The TCR of claim 1 or 2, wherein the Vβ further comprises the CDR1 and CDR2 amino acid sequences of TRBV7-8.

8. The TCR of claim 1, 2, or 7, wherein the Vβ comprises an amino acid sequence at least 90% identical to the amino acid sequence of TRBV7-8.

9. The TCR of any one of claims 1-8, wherein the Vα comprises a CDR3 amino acid sequence and the CDR1 and CDR2 amino acid sequences of the corresponding TRAV set forth in Table 1.

10. The TCR of claim 9, wherein the Vα comprises an amino acid sequence at least 90% identical to the amino acid sequence of the corresponding TRAV set forth in Table 1.

11. A T cell receptor (TCR) comprising a Vα and a Vβ, wherein the Vα comprises a CDR3 sequence of SEQ ID NO: 60, and the Vβ comprises a CDR3 sequence of SEQ ID NO: 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, or 134, wherein the TCR binds an epitope comprising the amino acid sequence of QVDYYGLYY (SEQ ID NO: 59) presented by HLA-A*01:01.

12. The TCR of claim 11, wherein the CDR3 sequence in the Vα comprises the amino acid sequence of SEQ ID NO: 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, or 133.

13. The TCR of claim 11 or 12, wherein the Vα further comprises the CDR1 and CDR2 amino acid sequences of TRAV21.

14. The TCR of any one of claims 11-[0059], wherein the Vα comprises an amino acid sequence at least 90% identical to the amino acid sequence of TRAV21.

15. The TCR of any one of claims 11-[0060], wherein the Vβ comprises a CDR3 amino acid sequence and the CDR1 and CDR2 amino acid sequences of the corresponding TRBV set forth in Table 2.

16. The TCR of claim [0060], wherein the Vβ comprises an amino acid sequence at least 90% identical to the amino acid sequence of the corresponding TRBV set forth in Table 2.

17. The TCR of any one of claims 1-[0060], wherein the TCR is isolated, non-naturally occurring, and/or engineered.

18. The TCR of claim 17, wherein the TCR is a soluble TCR.

19. The soluble TCR of claim 18, further comprising an alpha chain constant domain (Cα) and a beta chain constant domain (Cβ).

20. The soluble TCR of claim 19, comprising one or more mutations that stabilize the interaction between the Cα and the Cβ.

21. The soluble TCR of claim 20, wherein the Cα comprises a cysteine residue at position 48 corresponding to the TRAC amino acid sequence of SEQ ID NO: 209, and the CB comprises a cysteine residue at position 57 corresponding to the TRBC1 or TRBC2 amino acid sequence of SEQ ID NO: 210 or 211, respectively.

22. The soluble TCR of claim 21, wherein the cysteine residue at position 48 in the Cα and the cysteine residue at position 57 in the Cβform a disulfide bond.

23. The soluble TCR of any one of claims 20-22, wherein:
(a) the Cα comprises a phenylalanine residue at position 21, an isoleucine residue at position 32, and/or a threonine residue at position 72, corresponding to the TRAC amino acid sequence of SEQ ID NO: 209; and/or
(b) the Cβ comprises a lysine residue at position 18, an arginine residue at position 23, a proline residue at position 39, and/or an aspartic acid or glutamic acid at position 54, corresponding to the TRBC1 or TRBC2 amino acid sequence of SEQ ID NO: 210 or 211, respectively.

24. A TCR fusion protein comprising the soluble TCR of any one of claims 18-23 and a binding domain that binds a receptor on an outer surface of an immune cell, a cytotoxic agent, a detectable label, or a combination thereof.

25. The TCR fusion protein of claim 24, wherein the binding domain binds a receptor on an outer surface of a T cell.

26. The TCR fusion protein of claim 24 or 25, wherein the binding domain comprises an antibody, or an antigen-binding fragment thereof, that binds the receptor.

27. The TCR fusion protein of claim 25 or 26, wherein the receptor is selected from the group consisting of CD3, CD2, CD28, and CD8.

28. The TCR fusion protein of claim 27, wherein the binding domain comprises a heavy chain variable domain (VH) and a light chain variable domain (VL) of an anti-CD3 antibody.

29. The TCR fusion protein of any one of claims 24-28, further comprising a half-life extending domain.

30. The TCR fusion protein of claim 29, wherein the half-life extending domain comprises an antibody Fc region.

31. The TCR fusion protein of claim 30, wherein the antibody Fc region comprises a human IgG1 Fc region comprising one or more effector function silencing mutations, optionally at one or more of positions selected from 233, 234, 235, 236, 297, 327, 330, and 331, according to EU numbering.

32. The TCR fusion protein of claim 30, wherein the antibody Fc region comprises a human IgG4 or IgG2 Fc region.

33. A pharmaceutical composition comprising the TCR fusion protein of any one of claims 24-32 and a pharmaceutically acceptable carrier or excipient.

34. A method of treating cancer, the method comprising administering to a subject in need thereof an effective amount of the TCR fusion protein of any one of claims 24-32 or the pharmaceutical composition of claim 33.

35. One or more nucleic acids encoding the TCR or soluble TCR of any one of claims 1-23 or the TCR fusion protein of any one of claims 24-32.

36. A vector comprising the one or more nucleic acids of claim 35.

37. A cell comprising the one or more nucleic acids of claim 35 or the vector of claim 36.

38. A method of producing a TCR or TCR fusion protein, the method comprising incubating the cell of claim 37 under conditions to express the TCR or the TCR fusion protein.

39. An engineered immune cell comprising one or more exogenous nucleic acids that encode the TCR of any one of claims 1-18.

40. The engineered immune cell of claim 39, wherein the immune cell is a T cell.

41. The engineered immune cell of claim 40, wherein the T cell is a CD8⁺ T cell.

42. A pharmaceutical composition comprising the engineered immune cell of any one of claims 39-41 and a pharmaceutically acceptable carrier or excipient.

43. A method of producing an engineered immune cell, the method comprising contacting an immune cell with one or more nucleic acids that encode the TCR of any one of claims 1-18.

44. A method of treating cancer, the method comprising administering to a subject in need thereof an effective amount of the engineered immune cell of any one of claims 39-41 or the pharmaceutical composition of claim 42.

45. The method of claim 44, wherein the immune cell is autologous.

46. The method of claim 44, wherein the immune cell is obtained from a healthy donor.

47. The method of any one of claims 34 and 44-46, wherein the cancer is HPV-16 positive.


SEQUENCE LISTING (246 sequences)

SEQ ID NO: 1 - protein, 9 aa, synthetic construct
YSKNKVWEV

SEQ ID NO: 2 - protein, 14 aa, synthetic construct
  VARIANT 5: note = wherein X is L, V, I, S, or a peptide bond linking the amino acid in position 4 to amino acid in position 6
  VARIANT 6: note = X is R or F
  VARIANT 7: note = X is A, K, R, or a peptide bond linking the amino acid in position 6 to amino acid in position 8
  VARIANT 10: note = X is Q or W
  VARIANT 11: note = X is P or T
  VARIANT 13: note = X is H or Y
CASSXXXRBXXQXF

SEQ ID NO: 3 - protein, 16 aa, synthetic construct
CAMREGGTSGTYKYIF

SEQ ID NO: 4 - protein, 13 aa, synthetic construct
CASSLRRDQPQHF

SEQ ID NO: 5 - protein, 16 aa, synthetic construct
CAMREDAMDSSYKLIF

SEQ ID NO: 6 - protein, 13 aa, synthetic construct
CASSLRRDQPQHF

SEQ ID NO: 7 - protein, 16 aa, synthetic construct
CAMREASLSGTYKYIF

SEQ ID NO: 8 - protein, 13 aa, synthetic construct
CASSFKRDQPQHF

SEQ ID NO: 9 - protein, 16 aa, synthetic construct
CAMREGSLQGTYKYIF

SEQ ID NO: 10 - protein, 13 aa, synthetic construct
CASSIRRDQPQHF

SEQ ID NO: 11 - protein, 15 aa, synthetic construct
CAMRGVMDSSYKLIF

SEQ ID NO: 12 - protein, 13 aa, synthetic construct
CASSLRRDQPQHF

SEQ ID NO: 13 - protein, 13 aa, synthetic construct
CAENMDGNTPLVF

SEQ ID NO: 14 - protein, 13 aa, synthetic construct
CASSLRRDQPQHF

SEQ ID NO: 15 - protein, 16 aa, synthetic construct
CAMREGGTSGTYKYIF

SEQ ID NO: 16 - protein, 13 aa, synthetic construct
CASSLRRDQPQHF

SEQ ID NO: 17 - protein, 16 aa, synthetic construct
CAMREGSTSGTYKYIF

SEQ ID NO: 18 - protein, 13 aa, synthetic construct
CASSSRRDQPQHF

SEQ ID NO: 19 - protein, 15 aa, synthetic construct
CALSGQMDSSYKLIF

SEQ ID NO: 20 - protein, 13 aa, synthetic construct
CASSVRRDQPQHF

SEQ ID NO: 21 - protein, 11 aa, synthetic construct
CAGASDYKLSF

SEQ ID NO: 22 - protein, 13 aa, synthetic construct
CASSRRRNQPQHF

SEQ ID NO: 23 - protein, 15 aa, synthetic construct
CALSGTMDSSYKLIF

SEQ ID NO: 24 - protein, 13 aa, synthetic construct
CASSLRRDWPQHF

SEQ ID NO: 25 - protein, 13 aa, synthetic construct
CALTFNQGGKLIF

SEQ ID NO: 26 - protein, 14 aa, synthetic construct
CASSLRARDQPQHF

SEQ ID NO: 27 - protein, 16 aa, synthetic construct
CAMREGGSDSSYKLIF

SEQ ID NO: 28 - protein, 13 aa, synthetic construct
CASSLRRDQTQYF

SEQ ID NO: 29 - protein, 16 aa, synthetic construct
CAMREGGTSGTYKYIF

SEQ ID NO: 30 - protein, 13 aa, synthetic construct
CASSLRRDQPQHF

SEQ ID NO: 31 - DNA, 48 bp, synthetic construct
  source 1..48: mol_type = other DNA
tgtgcaatgagagagggcggtacctcaggaacctacaaatacatcttt

SEQ ID NO: 32 - DNA, 39 bp, synthetic construct
  source 1..39: mol_type = other DNA
tgtgccagcagcttacgacgcgatcagccccagcatttt

SEQ ID NO: 33 - DNA, 48 bp, synthetic construct
  source 1..48: mol_type = other DNA
tgtgcaatgagagaggatgcgatggatagcagctataaattgatcttc

SEQ ID NO: 34 - DNA, 39 bp, synthetic construct
  source 1..39: mol_type = other DNA
tgtgccagcagcttgaggagagatcagccccagcatttt

SEQ ID NO: 35 - DNA, 48 bp, synthetic construct
  source 1..48: mol_type = other DNA
tgtgcaatgagagaggcttccctctcaggaacctacaaatacatcttt

SEQ ID NO: 36 - DNA, 39 bp, synthetic construct
  source 1..39: mol_type = other DNA
tgtgccagcagcttcaagagggatcagccccagcatttt

SEQ ID NO: 37 - DNA, 48 bp, synthetic construct
  source 1..48: mol_type = other DNA
tgtgcaatgagagagggcagtctacagggaacctacaaatacatcttt

SEQ ID NO: 38 - DNA, 39 bp, synthetic construct
  source 1..39: mol_type = other DNA
tgtgccagcagcatacgccgggatcagccccagcatttt

SEQ ID NO: 39 - DNA, 45 bp, synthetic construct
  source 1..45: mol_type = other DNA
tgtgcaatgagaggggtgatggatagcagctataaattgatcttc

SEQ ID NO: 40 - DNA, 39 bp, synthetic construct
  source 1..39: mol_type = other DNA
tgtgccagcagcttacggagggatcagccccagcatttt

SEQ ID NO: 41 - DNA, 39 bp, synthetic construct
  source 1..39: mol_type = other DNA
tgtgcagagaatatggacggaaacacacctcttgtcttt

SEQ ID NO: 42 - DNA, 39 bp, synthetic construct
  source 1..39: mol_type = other DNA
tgtgccagcagcttaaggagagatcagccccagcatttt

SEQ ID NO: 43 - DNA, 48 bp, synthetic construct; identical to SEQ ID NO: 31
  source 1..48: mol_type = other DNA

SEQ ID NO: 44 - DNA, 39 bp, synthetic construct
  source 1..39: mol_type = other DNA
tgtgccagcagcttacgtcgggatcagccccagcatttt

SEQ ID NO: 45 - DNA, 48 bp, synthetic construct
  source 1..48: mol_type = other DNA
tgtgcaatgagagagggtagtacctcaggaacctacaaatacatcttt

SEQ ID NO: 46 - DNA, 39 bp, synthetic construct
  source 1..39: mol_type = other DNA
tgtgccagcagctcacgcagagatcagccccagcatttt

SEQ ID NO: 47 - DNA, 45 bp, synthetic construct
  source 1..45: mol_type = other DNA
tgtgctctgagcgggcagatggatagcagctataaattgatcttc

SEQ ID NO: 48 - DNA, 39 bp, synthetic construct
  source 1..39: mol_type = other DNA
tgtgccagcagcgtacgcagggatcagccccagcatttt

SEQ ID NO: 49 - DNA, 33 bp, synthetic construct
  source 1..33: mol_type = other DNA
tgtgcaggagctagcgactacaagctcagcttt

SEQ ID NO: 50 - DNA, 39 bp, synthetic construct
  source 1..39: mol_type = other DNA
tgtgccagcagccggcgccgaaatcagccccagcatttt

SEQ ID NO: 51 - DNA, 45 bp, synthetic construct
  source 1..45: mol_type = other DNA
tgtgctctgagtgggactatggatagcagctataaattgatcttc

SEQ ID NO: 52 - DNA, 39 bp, synthetic construct
  source 1..39: mol_type = other DNA
tgtgccagcagcttacggagggactggccccagcatttt

SEQ ID NO: 53 - DNA, 39 bp, synthetic construct
  source 1..39: mol_type = other DNA
tgtgctctgacttttaaccagggaggaaagcttatcttc

SEQ ID NO: 54 - DNA, 42 bp, synthetic construct
  source 1..42: mol_type = other DNA
tgtgccagcagcttacgggcaagggatcagccccagcatttt

SEQ ID NO: 55 - DNA, 48 bp, synthetic construct
  source 1..48: mol_type = other DNA
tgtgcaatgagagagggcgggtcggatagcagctataaattgatcttc

SEQ ID NO: 56 - DNA, 39 bp, synthetic construct
  source 1..39: mol_type = other DNA
tgtgccagcagcttaaggcgggaccagacccagtacttc

SEQ ID NO: 57 - DNA, 48 bp, synthetic construct
  source 1..48: mol_type = other DNA
tgtgcaatgagagagggcgggacctcaggaacctacaaatacatcttt

SEQ ID NO: 58 - DNA, 39 bp, synthetic construct
  source 1..39: mol_type = other DNA
tgtgccagcagcttaagaagggaccagccccagcatttt

SEQ ID NO: 59 - protein, 9 aa, synthetic construct
QVDYYGLYY

SEQ ID NO: 60 - protein, 12 aa, synthetic construct
  VARIANT 3: note = wherein X is V or P
CAXBTGGFKTIF

SEQ ID NO: 61 - protein, 12 aa, synthetic construct
CAVDTGGFKTIF

SEQ ID NO: 62 - protein, 14 aa, synthetic construct
CASSPADRGDTIYF

SEQ ID NO: 63 - protein, 12 aa, synthetic construct
CAVNTGGFKTIF

SEQ ID NO: 64 - protein, 15 aa, synthetic construct
CASSGTGMANEKLFF

SEQ ID NO: 65 - protein, 12 aa, synthetic construct
CAVDTGGFKTIF

SEQ ID NO: 66 - protein, 14 aa, synthetic construct
CASSLASGDYGYTF

SEQ ID NO: 67 - protein, 12 aa, synthetic construct
CAVDTGGFKTIF

SEQ ID NO: 68 - protein, 13 aa, synthetic construct
CASSSADGAGYTF

SEQ ID NO: 69 - protein, 12 aa, synthetic construct
CAVDTGGFKTIF

SEQ ID NO: 70 - protein, 14 aa, synthetic construct
CASSLADGNEKLFF

SEQ ID NO: 71 - protein, 12 aa, synthetic construct
CAVDTGGFKTIF

SEQ ID NO: 72 - protein, 14 aa, synthetic construct
CASTSGDGNYGYTF

SEQ ID NO: 73 - protein, 12 aa, synthetic construct
CAVNTGGFKTIF

SEQ ID NO: 74 - protein, 14 aa, synthetic construct
CASTSGDGNEKLFF

SEQ ID NO: 75 - protein, 12 aa, synthetic construct
CAVDTGGFKTIF

SEQ ID NO: 76 - protein, 15 aa, synthetic construct
CASSSGDRDNYGYTF

SEQ ID NO: 77 - protein, 12 aa, synthetic construct
CAVDTGGFKTIF

SEQ ID NO: 78 - protein, 14 aa, synthetic construct
CASSDLSGNQPQHF

SEQ ID NO: 79 - protein, 12 aa, synthetic construct
CAVDTGGFKTIF

SEQ ID NO: 80 - protein, 15 aa, synthetic construct
CASSLDLSGANVLTF

SEQ ID NO: 81 - protein, 12 aa, synthetic construct
CAVDTGGFKTIF

SEQ ID NO: 82 - protein, 14 aa, synthetic construct
CASSSLDGNTEAFF

SEQ ID NO: 83 - protein, 12 aa, synthetic construct
CAVDTGGFKTIF

SEQ ID NO: 84 - protein, 15 aa, synthetic construct
CSAREGRVGNSPLHF

SEQ ID NO: 85 - protein, 12 aa, synthetic construct
CAVDTGGFKTIF

SEQ ID NO: 86 - protein, 13 aa, synthetic construct
CSVGGQLNQPQHF

SEQ ID NO: 87 - protein, 12 aa, synthetic construct
CAVNTGGFKTIF

SEQ ID NO: 88 - protein, 15 aa, synthetic construct
CASSGTGLSNEKLFF

SEQ ID NO: 89 - protein, 12 aa, synthetic construct
CAPNTGGFKTIF

SEQ ID NO: 90 - protein, 15 aa, synthetic construct
CASSQEPSGGYEQYF

SEQ ID NO: 91 - protein, 12 aa, synthetic construct
CAVNTGGFKTIF

SEQ ID NO: 92 - protein, 13 aa, synthetic construct
CASSLRVYETQYF

SEQ ID NO: 93 - protein, 12 aa, synthetic construct
CAVNTGGFKTIF

SEQ ID NO: 94 - protein, 15 aa, synthetic construct
CASSLSWDRDRSYTF

SEQ ID NO: 95 - protein, 12 aa, synthetic construct
CAVDTGGFKTIF

SEQ ID NO: 96 - protein, 16 aa, synthetic construct
CSAEGTSGKVGYEQFF

SEQ ID NO: 97 - protein, 12 aa, synthetic construct
CAVNTGGFKTIF

SEQ ID NO: 98 - protein, 16 aa, synthetic construct
CASSPSRPRVEETQYF

SEQ ID NO: 99 - protein, 12 aa, synthetic construct
CAVNTGGFKTIF

SEQ ID NO: 100 - protein, 15 aa, synthetic construct
CASSSQGTLGVPLHF

SEQ ID NO: 101 - protein, 12 aa, synthetic construct
CAVNTGGFKTIF

SEQ ID NO: 102 - protein, 15 aa, synthetic construct
CASSTTGLANEKLFF

SEQ ID NO: 103 - protein, 12 aa, synthetic construct
CAVNTGGFKTIF

SEQ ID NO: 104 - protein, 14 aa, synthetic construct
CASTSGDGNEKLFF

SEQ ID NO: 105 - protein, 12 aa, synthetic construct
CAVNTGGFKTIF

SEQ ID NO: 106 - protein, 13 aa, synthetic construct
CASSPAWLDEQYF

SEQ ID NO: 107 - protein, 12 aa, synthetic construct
CAVDTGGFKTIF

SEQ ID NO: 108 - protein, 15 aa, synthetic construct
CASSSGDRDNYGYTF

SEQ ID NO: 109 - protein, 12 aa, synthetic construct
CAVDTGGFKTIF

SEQ ID NO: 110 - protein, 12 aa, synthetic construct
CASSYDRYEQYF

SEQ ID NO: 111 - protein, 12 aa, synthetic construct
CAVDTGGFKTIF

SEQ ID NO: 112 - protein, 14 aa, synthetic construct
CASSYASGDYGYTF

SEQ ID NO: 113 - protein, 12 aa, synthetic construct
CAVDTGGFKTIF

SEQ ID NO: 114 - protein, 13 aa, synthetic construct
CASALYSTDTQYF

SEQ ID NO: 115 - protein, 12 aa, synthetic construct
CAVDTGGFKTIF

SEQ ID NO: 116 - protein, 15 aa, synthetic construct
CASSQEGAPAGELFF

SEQ ID NO: 117 - protein, 12 aa, synthetic construct
CAVDTGGFKTIF

SEQ ID NO: 118 - protein, 14 aa, synthetic construct
CASSPGDRSYGYTF

SEQ ID NO: 119 - protein, 12 aa, synthetic construct
CAVDTGGFKTIF

SEQ ID NO: 120 - protein, 14 aa, synthetic construct
CASSPADRGDTIYF

SEQ ID NO: 121 - protein, 12 aa, synthetic construct
CAVDTGGFKTIF

SEQ ID NO: 122 - protein, 14 aa, synthetic construct
CASSLSDGNYGYTF

SEQ ID NO: 123 - protein, 12 aa, synthetic construct
CAVDTGGFKTIF

SEQ ID NO: 124 - protein, 16 aa, synthetic construct
CASSLRGLAIPYEQYV

SEQ ID NO: 125 - protein, 12 aa, synthetic construct
CAVDTGGFKTIF

SEQ ID NO: 126 - protein, 14 aa, synthetic construct
CASSLGDGNYGYTF

SEQ ID NO: 127 - protein, 12 aa, synthetic construct
CAVDTGGFKTIF

SEQ ID NO: 128 - protein, 14 aa, synthetic construct
CASSLADGNYGYTF

SEQ ID NO: 129 - protein, 12 aa, synthetic construct
CAVDTGGFKTIF

SEQ ID NO: 130 - protein, 14 aa, synthetic construct
CASSLADGNEKLFF

SEQ ID NO: 131 - protein, 12 aa, synthetic construct
CAVDTGGFKTIF

SEQ ID NO: 132 - protein, 15 aa, synthetic construct
CASSFDLSGANVLTF

SEQ ID NO: 133 - protein, 12 aa, synthetic construct
CAVNTGGFKTIF

SEQ ID NO: 134 - protein, 14 aa, synthetic construct
CSARDRQGDEKLFF

SEQ ID NO: 135 - DNA, 36 bp, synthetic construct
  source 1..36: mol_type = other DNA
tgtgctgtcgatactggaggcttcaaaactatcttt

SEQ ID NO: 136 - DNA, 42 bp, synthetic construct
  source 1..42: mol_type = other DNA
tgtgccagcagtcccgcggacaggggtgacaccatatatttt

SEQ ID NO: 137 - DNA, 36 bp, synthetic construct
  source 1..36: mol_type = other DNA
tgtgctgtcaatactggaggcttcaaaactatcttt

SEQ ID NO: 138 - DNA, 45 bp, synthetic construct
  source 1..45: mol_type = other DNA
tgtgccagcagtggaacagggatggctaatgaaaaactgtttttt

SEQ ID NO: 139 - DNA, 36 bp, synthetic construct
  source 1..36: mol_type = other DNA
tgtgctgtggatactggaggcttcaaaactatcttt

SEQ ID NO: 140 - DNA, 42 bp, synthetic construct
  source 1..42: mol_type = other DNA
tgtgccagcagtttggcctctggggattatggctacaccttc

SEQ ID NO: 141 - DNA, 36 bp, synthetic construct
  source 1..36: mol_type = other DNA
tgtgctgttgatactggaggcttcaaaactatcttt

SEQ ID NO: 142 - DNA, 39 bp, synthetic construct
  source 1..39: mol_type = other DNA
tgtgccagcagttcggcggacggcgcgggctacaccttc

SEQ ID NO: 143 - DNA, 36 bp, synthetic construct; identical to SEQ ID NO: 139
  source 1..36: mol_type = other DNA

SEQ ID NO: 144 - DNA, 42 bp, synthetic construct
  source 1..42: mol_type = other DNA
tgtgccagcagtttggcagacggtaatgaaaaactgtttttt

SEQ ID NO: 145 - DNA, 36 bp, synthetic construct
  source 1..36: mol_type = other DNA
tgtgctgtggacactggaggcttcaaaactatcttt

SEQ ID NO: 146 - DNA, 42 bp, synthetic construct
  source 1..42: mol_type = other DNA
tgtgccagcacgtcaggggacggaaactatggctacaccttc

SEQ ID NO: 147 - DNA, 36 bp, synthetic construct
  source 1..36: mol_type = other DNA
tgtgctgttaatactggaggcttcaaaactatcttt

SEQ ID NO: 148 - DNA, 42 bp, synthetic construct
  source 1..42: mol_type = other DNA
tgtgccagcacctcgggggatggtaatgaaaaactgtttttt

SEQ ID NO: 149 - DNA, 36 bp, synthetic construct; identical to SEQ ID NO: 139
  source 1..36: mol_type = other DNA

SEQ ID NO: 150 - DNA, 45 bp, synthetic construct
  source 1..45: mol_type = other DNA
tgtgccagcagttcaggagacagggacaactatggctacaccttc

SEQ ID NO: 151 - DNA, 36 bp, synthetic construct; identical to SEQ ID NO: 135
  source 1..36: mol_type = other DNA

SEQ ID NO: 152 - DNA, 42 bp, synthetic construct
  source 1..42: mol_type = other DNA
tgtgccagcagtgacctttcagggaatcagccccagcatttt

SEQ ID NO: 153 - DNA, 36 bp, synthetic construct; identical to SEQ ID NO: 145
  source 1..36: mol_type = other DNA

SEQ ID NO: 154 - DNA, 45 bp, synthetic construct
  source 1..45: mol_type = other DNA
tgtgccagcagtttagatctgtctggggccaacgtcctgactttc

SEQ ID NO: 155 - DNA, 36 bp, synthetic construct; identical to SEQ ID NO: 139
  source 1..36: mol_type = other DNA

SEQ ID NO: 156 - DNA, 42 bp, synthetic construct
  source 1..42: mol_type = other DNA
tgtgccagcagttccctggacggcaacactgaagctttcttt

SEQ ID NO: 157 - DNA, 36 bp, synthetic construct; identical to SEQ ID NO: 135
  source 1..36: mol_type = other DNA

SEQ ID NO: 158 - DNA, 45 bp, synthetic construct
  source 1..45: mol_type = other DNA
tgcagtgctagagagggcagggtggggaattcacccctccacttt

SEQ ID NO: 159 - DNA, 36 bp, synthetic construct; identical to SEQ ID NO: 139
  source 1..36: mol_type = other DNA

SEQ ID NO: 160 - DNA, 39 bp, synthetic construct
  source 1..39: mol_type = other DNA
tgcagcgttgggggacagctcaatcagccccagcatttt

SEQ ID NO: 161 - DNA, 36 bp, synthetic construct
  source 1..36: mol_type = other DNA
tgtgctgtgaatactggaggcttcaaaactatcttt

SEQ ID NO: 162 - DNA, 45 bp, synthetic construct
  source 1..45: mol_type = other DNA
tgtgccagcagtgggacagggcttagtaatgaaaaactgtttttt

SEQ ID NO: 163 - DNA, 36 bp, synthetic construct
  source 1..36: mol_type = other DNA
tgtgctccaaatactggaggcttcaaaactatcttt

SEQ ID NO: 164 - DNA, 45 bp, synthetic construct
  source 1..45: mol_type = other DNA
tgcgccagcagccaagaacctagcgggggctacgagcagtacttc

SEQ ID NO: 165 - DNA, 36 bp, synthetic construct; identical to SEQ ID NO: 137
  source 1..36: mol_type = other DNA

SEQ ID NO: 166 - DNA, 39 bp, synthetic construct
  source 1..39: mol_type = other DNA
tgtgccagcagcttaagggtttacgagacccagtacttc

SEQ ID NO: 167 - DNA, 36 bp, synthetic construct; identical to SEQ ID NO: 137
  source 1..36: mol_type = other DNA

SEQ ID NO: 168 - DNA, 45 bp, synthetic construct
  source 1..45: mol_type = other DNA
tgtgccagcagcctaagttgggacagggacagaagctacaccttc

SEQ ID NO: 169 - DNA, 36 bp, synthetic construct; identical to SEQ ID NO: 141
  source 1..36: mol_type = other DNA

SEQ ID NO: 170 - DNA, 48 bp, synthetic construct
  source 1..48: mol_type = other DNA
tgcagtgcggaagggactagcgggaaggtagggtatgagcagttcttc

SEQ ID NO: 171 - DNA, 36 bp, synthetic construct; identical to SEQ ID NO: 137
  source 1..36: mol_type = other DNA

SEQ ID NO: 172 - DNA, 48 bp, synthetic construct
  source 1..48: mol_type = other DNA
tgtgccagcagtccctcccgacccagggtcgaggagacccagtacttc

SEQ ID NO: 173 - DNA, 36 bp, synthetic construct; identical to SEQ ID NO: 147
  source 1..36: mol_type = other DNA

SEQ ID NO: 174 - DNA, 45 bp, synthetic construct
  source 1..45: mol_type = other DNA
tgtgccagcagttcacaggggaccctcggggttcccctccacttt

SEQ ID NO: 175 - DNA, 36 bp, synthetic construct
  source 1..36: mol_type = other DNA
tgtgctgtaaatactggaggcttcaaaactatcttt

SEQ ID NO: 176 - DNA, 45 bp, synthetic construct
  source 1..45: mol_type = other DNA
tgtgccagcagtacgacagggttggctaatgaaaaactgtttttt

SEQ ID NO: 177 - DNA, 36 bp, synthetic construct; identical to SEQ ID NO: 147
  source 1..36: mol_type = other DNA

SEQ ID NO: 178 - DNA, 42 bp, synthetic construct; identical to SEQ ID NO: 148
  source 1..42: mol_type = other DNA

SEQ ID NO: 179 - DNA, 36 bp, synthetic construct; identical to SEQ ID NO: 161
  source 1..36: mol_type = other DNA

SEQ ID NO: 180 - DNA, 39 bp, synthetic construct
  source 1..39: mol_type = other DNA
tgcgccagcagccccgcatggttggacgagcagtacttc

SEQ ID NO: 181 - DNA, 36 bp, synthetic construct; identical to SEQ ID NO: 139
  source 1..36: mol_type = other DNA

SEQ ID NO: 182 - DNA, 45 bp, synthetic construct
  source 1..45: mol_type = other DNA
tgtgccagcagtagcggggacagggataactatggctacaccttc

SEQ ID NO: 183 - DNA, 36 bp, synthetic construct; identical to SEQ ID NO: 135
  source 1..36: mol_type = other DNA

SEQ ID NO: 184 - DNA, 36 bp, synthetic construct
  source 1..36: mol_type = other DNA
tgtgccagcagttacgaccgctacgagcagtacttc

SEQ ID NO: 185 - DNA, 36 bp, synthetic construct; identical to SEQ ID NO: 139
  source 1..36: mol_type = other DNA

SEQ ID NO: 186 - DNA, 42 bp, synthetic construct
  source 1..42: mol_type = other DNA
tgtgccagcagttacgcatctggggactatggctacaccttc

SEQ ID NO: 187 - DNA, 36 bp, synthetic construct; identical to SEQ ID NO: 139
  source 1..36: mol_type = other DNA

SEQ ID NO: 188 - DNA, 39 bp, synthetic construct
  source 1..39: mol_type = other DNA
tgtgccagcgcgctctatagcacagatacgcagtatttt

SEQ ID NO: 189 - DNA, 36 bp, synthetic construct; identical to SEQ ID NO: 135
  source 1..36: mol_type = other DNA

SEQ ID NO: 190 - DNA, 45 bp, synthetic construct
  source 1..45: mol_type = other DNA
tgcgccagcagccaagaaggggccccagccggggagctgtttttt

SEQ ID NO: 191 - DNA, 36 bp, synthetic construct; identical to SEQ ID NO: 135
  source 1..36: mol_type = other DNA

SEQ ID NO: 192 - DNA, 42 bp, synthetic construct
  source 1..42: mol_type = other DNA
tgtgccagcagccccggggacaggagctatggctacaccttc

SEQ ID NO: 193 - DNA, 36 bp, synthetic construct; identical to SEQ ID NO: 135
  source 1..36: mol_type = other DNA

SEQ ID NO: 194 - DNA, 42 bp, synthetic construct; identical to SEQ ID NO: 136
  source 1..42: mol_type = other DNA

SEQ ID NO: 195 - DNA, 36 bp, synthetic construct
  source 1..36: mol_type = other DNA
tgtgccgtagatactggaggcttcaaaactatcttt

SEQ ID NO: 196 - DNA, 42 bp, synthetic construct
  source 1..42: mol_type = other DNA
tgtgccagcagtttatcggacggaaactatggctacaccttc

SEQ ID NO: 197 - DNA, 36 bp, synthetic construct; identical to SEQ ID NO: 141
  source 1..36: mol_type = other DNA

SEQ ID NO: 198 - DNA, 48 bp, synthetic construct
  source 1..48: mol_type = other DNA
tgtgccagcagtttgcgtggactagcgatcccctacgagcagtacgtc

SEQ ID NO: 199 - DNA, 36 bp, synthetic construct; identical to SEQ ID NO: 139
  source 1..36: mol_type = other DNA

SEQ ID NO: 200 - DNA, 42 bp, synthetic construct
  source 1..42: mol_type = other DNA
tgtgccagcagtttaggggacgggaactatggctacaccttc

SEQ ID NO: 201 - DNA, 36 bp, synthetic construct
  source 1..36: mol_type = other DNA
tgtgctgtagatactggaggcttcaaaactatcttt

SEQ ID NO: 202 - DNA, 42 bp, synthetic construct
  source 1..42: mol_type = other DNA
tgtgccagcagtttagcagacggaaactatggctacaccttc

SEQ ID NO: 203 - DNA, 36 bp, synthetic construct; identical to SEQ ID NO: 139
  source 1..36: mol_type = other DNA

SEQ ID NO: 204 - DNA, 42 bp, synthetic construct; identical to SEQ ID NO: 144
  source 1..42: mol_type = other DNA

SEQ ID NO: 205 - DNA, 36 bp, synthetic construct; identical to SEQ ID NO: 141
  source 1..36: mol_type = other DNA

SEQ ID NO: 206 - DNA, 45 bp, synthetic construct
  source 1..45: mol_type = other DNA
tgtgccagcagtttcgatctctctggggccaacgtcctgactttc

SEQ ID NO: 207 - DNA, 36 bp, synthetic construct; identical to SEQ ID NO: 161
  source 1..36: mol_type = other DNA

SEQ ID NO: 208 - DNA, 42 bp, synthetic construct
  source 1..42: mol_type = other DNA
tgcagtgctagagaccggcagggggatgaaaaactgtttttt

SEQ ID NO: 209 - protein, 141 aa, synthetic construct
DIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 210 - protein, 177 aa, synthetic construct
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 211 - protein, 179 aa, synthetic construct
EDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 212 - protein, 253 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 213 - protein, 107 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 214 - protein, 122 aa, synthetic construct [sequence omitted]

SEQ ID NO: 215 - protein, 11 aa, synthetic construct
RASQDIRNYLN

SEQ ID NO: 216 - protein, 7 aa, synthetic construct
YTSRLES

SEQ ID NO: 217 - protein, 9 aa, synthetic construct
QQGNTLPWT

SEQ ID NO: 218 - protein, 5 aa, synthetic construct
GYTMN

SEQ ID NO: 219 - protein, 17 aa, synthetic construct
LINPYKGVSTYNQKFKD

SEQ ID NO: 220 - protein, 13 aa, synthetic construct
SGYYGDSDWYFDV

SEQ ID NO: 221 - protein, 109 aa, synthetic construct
QAVVTQESALTTSPGETVTLTCRSSTGAVTTSNYANWVQEKPDHLFTGLIGGTNFRAPGVPARFSGSLIGDKAALTITGAQTEDEAIYFCALWYSNLWVFGGGTKLTVL

SEQ ID NO: 222 - protein, 124 aa, synthetic construct
VQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 223 - protein, 14 aa, synthetic construct
RSSTGAVTTSNYAN

SEQ ID NO: 224 - protein, 7 aa, synthetic construct
GTNFRAP

SEQ ID NO: 225 - protein, 9 aa, synthetic construct
ALWYSNLWV

SEQ ID NO: 226 - protein, 5 aa, synthetic construct
TYAMN

SEQ ID NO: 227 - protein, 19 aa, synthetic construct
RIRSKYNNYATYYADSVKD

SEQ ID NO: 228 - protein, 14 aa, synthetic construct
HGNFGNSYVSWFAY

SEQ ID NO: 229 - protein, 107 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 230 - protein, 118 aa, synthetic construct
QVQLQQSGAELARPGASVKMSCKASGYTFTRYTMHWVKQRPQGLEWIGYINPSRGYTNTNQKFKDKATLTTDKSSSTAYMQLSSLTSEDSAVYYCARYYDDHYCLDYWGQGTTLTVSS

SEQ ID NO: 231 - protein, 10 aa, synthetic construct
SASSSVSYMN

SEQ ID NO: 232 - protein, 7 aa, synthetic construct
DTSKLAS

SEQ ID NO: 233 - protein, 9 aa, synthetic construct
QQWSSNPFT

SEQ ID NO: 234 - protein, 5 aa, synthetic construct
RYTMH

SEQ ID NO: 235 - protein, 17 aa, synthetic construct
YINPSRGYTNTNQKFKD

SEQ ID NO: 236 - protein, 10 aa, synthetic construct
YYDDHYCLDY

SEQ ID NO: 237 - protein, 45 aa, synthetic construct
LKEAKEKAIEELKKAGITSDYYFDLINKAKTVEGVNALKDEILKA

SEQ ID NO: 238 - protein, 5 aa, synthetic construct
GGGGS

SEQ ID NO: 239 - protein, 5 aa, synthetic construct
GGGSG

SEQ ID NO: 240 - protein, 5 aa, synthetic construct
GGSGG

SEQ ID NO: 241 - protein, 5 aa, synthetic construct
GSGGG

SEQ ID NO: 242 - protein, 6 aa, synthetic construct
GSGGGP

SEQ ID NO: 243 - protein, 5 aa, synthetic construct
GGEPS

SEQ ID NO: 244 - protein, 7 aa, synthetic construct
GGEGGGP

SEQ ID NO: 245 - protein, 12 aa, synthetic construct
GGEGGGSEGGGS

SEQ ID NO: 246 - protein, 24 aa, synthetic construct
GGGGSGGGGSGGGGSGGGGSGGGS